(19) United States
(12) Patent Application Publication
(10) Pub. No.: US 2019/0125829 A1
(43) Pub. Date: May 2, 2019

(54) METHOD FOR PREVENTING OBESITY-INDUCED FATTY LIVER BY INHIBITING KCTD17

(71) Applicant: The Trustees of Columbia University in the City of New York, New York, NY (US)

(72) Inventors: Utpal Pajvani, Leonia, NJ (US); Kyeongjin Kim, New York, NY (US)

(73) Assignee: The Trustees of Columbia University in the City of New York, New York, NY (US)

(21) Appl. No.: 16/096,573

(22) PCT Filed: Apr. 28, 2017
(86) PCT No.: PCT/US2017/030102

§ 371 Date: Oct. 25, 2018

Related U.S. Application Data
(63) Continuation of application No. 15/143,305, filed on Apr. 29, 2016, now abandoned.
(60) Provisional application No. 62/267,632, filed on Dec. 15, 2015.
(60) Provisional application No. 62/234,505, filed on Sep. 29, 2015.
(60) Provisional application No. 62/155,696, filed on May 1, 2015.

(51) Int. Cl. A61K 38/17; G01N 33/50; G01N 33/74; G01N 33/92; C12Q 1/42; C12N 15/113

(52) U.S. Cl. CPC A61K 38/1709; G01N 33/5067; G01N 33/74; G01N 33/92; C12Q 1/42; C12N 15/86; C12N 15/1137; G01N 2405/02; G01N 2333/916; G01N 2333/605; C12N 2310/20; C12N 15/1138

(57) ABSTRACT

The present invention provides methods for reducing a subject's hepatic and plasma triglyceride levels comprising administering to a subject in need thereof a pharmaceutical composition comprising a pharmaceutical carrier and a compound that decreases KCTD17 expression in liver cells in an amount effective to reduce the subject's hepatic and plasma triglyceride levels.

This invention was made with government support under grant number DK093604 awarded by the National Institutes of Health. The government has certain rights in the invention.

This application claims the priority of U.S. patent application Ser. No. 15/143,305, filed Apr. 29, 2016, the contents of which are hereby incorporated by reference.

This application incorporates-by-reference nucleotide and/or amino acid sequences which are present in the file named “170428_0575_89275_A_PCT_SequenceListing_MW.txt,” which is 28 kilobytes in size, and which was created Apr. 28, 2017 in the IBM-PC machine format, having an operating system compatibility with MS-Windows, which is contained in the text file filed Apr. 28, 2017 as part of this application.

Throughout this application various publications are referred to by first author and year of publication. Full citations of these references can be found following the Examples. The disclosures of these publications in their entireties are hereby incorporated by reference into this application in order to more fully describe the state of the art to which this invention pertains.

### BACKGROUND OF THE INVENTION

Obesity has reached epidemic status in the United States—the Centers for Disease Control has stated that more than ⅓ of American adults are obese, and estimated the medical costs attributable to obesity at $147 billion in 2008, a number that is likely to be significantly higher today and in the future.

Obesity-induced metabolic dysfunction manifests as multiple chronic medical conditions, including Type 2 Diabetes (T2D) and Nonalcoholic fatty liver disease (NAFLD) (Ford et al., 2002). NAFLD is the result of compensatory hyperinsulinemia and hepatic de novo lipogenesis. NAFLD contributes to the overall cardiovascular risk of obesity (Villanova et al., 2005), but is also the most common chronic liver disease, predisposing to cirrhosis and hepatocellular carcinoma (Dowman et al., 2011).

Obesity leads to insulin resistance, which begets the hyperglycemia of T2D. In a parallel but poorly understood process, compensatory hyperinsulinemia drives hepatic de novo lipogenesis, mediated in part by the nutrient-sensitive mechanistic target of rapamycin (mTOR) pathway, which predisposes excessive liver fat or NAFLD. The presence of NAFLD increases underlying insulin resistence—this vicious cycle results in exacerbations of both T2D and NAFLD, which show independent associations with cardiovascular disease and all-cause mortality.

Aging and obesity are well-established risk factors for NAFLD (Slawik and Vidal-Puig, 2006), but the molecular mechanism underlying this risk is poorly defined, precluding specific pharmacologic strategies to target excess hepatic triglycerides (TG).

There is no approved pharmacologic therapy for NAFLD—the only clinical recourse is liver transplantation; current projections suggest that NAFLD will be the leading cause for liver transplantation by 2020, a conundrum as available organs are already limiting. Novel pathways are sought to both further our understanding of the pathophysiology, as well as provide new pharmacologic targets to assist in out management of obesity-related morbidity and mortality.

Thus, new therapies are needed.

### SUMMARY OF THE INVENTION

The present invention provides a method of reducing a subject's hepatic and plasma triglyceride levels comprising administering to a subject in need thereof a pharmaceutical composition comprising a pharmaceutical carrier and a compound that decreases KCTD17 expression in liver cells in an amount effective to reduce the subject's hepatic and plasma triglyceride levels.

The present invention provides a method of reducing a subject's hepatic and plasma triglyceride levels comprising administering to a subject in need thereof a pharmaceutical composition comprising a pharmaceutical carrier and a compound that increases PHLPP2 in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that increases free Raptor in liver cells in an amount effective to reduce the subject's hepatic and plasma triglyceride levels.

The present invention also provides a process for determining the amount of free Raptor in a subject's liver comprising:
a) obtaining a biological sample comprising liver cells of the subject;
b) separating free Raptor and mTORC1-associated Raptor in the sample; and
c) determining the amount of free Raptor in the sample.

The present invention also provides a method of reducing a subject's hepatic and plasma triglyceride levels comprising administering to a subject in need thereof a pharmaceutical composition comprising a pharmaceutical carrier and a compound that prevents PHLPP2 degradation in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that inhibits Glucagon signaling in liver cells in an amount effective to reduce the subject's hepatic and plasma triglyceride levels.

The present invention also provides a process for determining the amount of KCTD17 expression in a subject's liver comprising:
a) obtaining a biological sample comprising liver cells of the subject;
b) determining the amount of KCTD17 mRNA in the sample.

The present invention also provides a process for diagnosing whether a subject is afflicted with increased KCTD17 expression comprising:
a) determining the amount of KCTD17 in the subject according to the process of claim 31;
b) determining the amount of KCTD17 in a reference subject according to the process of claim 31; and
c) diagnosing the subject to be afflicted with increased KCTD17 expression if the amount of KCTD17 expression in step (a) is substantially increased compared to the amount of KCTD17 expression in step (b).

The present invention also provides a method of treating a subject afflicted with elevated triglyceride levels comprising administering to the subject a pharmaceutical composition comprising a pharmaceutical carrier and a compound that decreases KCTD17 expression in liver cells in an amount effective to reduce the subject's hepatic and plasma triglyceride levels.

The present invention also provides a method of treating a subject afflicted with elevated triglyceride levels comprising administering to the subject a pharmaceutical composition comprising a pharmaceutical carrier and a compound that decreases PHLPP2 phosphorylation at Serine 1119 and Serine 1210 residues in liver cells in an amount effective to reduce the subject's hepatic and plasma triglyceride levels.

The present invention also provides a method of treating a subject at risk of developing elevated triglyceride levels comprising administering to the subject a pharmaceutical composition comprising a pharmaceutical carrier and a compound that decreases KCTD17 expression in liver cells in an amount effective to reduce the subject's hepatic and plasma triglyceride levels.

The present invention also provides a method of treating a subject at risk of developing elevated triglyceride levels comprising administering to the subject a pharmaceutical composition comprising a pharmaceutical carrier and a compound that increases PHLPP2 phosphorylation at Serine 1119 and Serine 1210 residues in liver cells in an amount effective to reduce the subject's hepatic and plasma triglyceride levels.

### BRIEF DESCRIPTION OF THE DRAWINGS

FIG. 1. Identification of PHLPP2 protein modification. C57BL/6 mice were injected with GFP (control) or HA/Flag/PHLPP2 adenovirus and sacrificed on day 5 after overnight fast, 2 hour re-fed, or 12 hour re-fed. Immunoprecipitation (IP) with anti-Flag (harsher condition) and liquid chromatography-tandem mass spectrometry (LC/MS-MS) were performed.

FIG. 2. Phosphorylation sites identified in PHLPP2.

FIG. 3. BasePeak Chromatogram. Overlay if base peak chromatogram of trypsin digest of the bottom band (enriched in PHLPP2). To no surprise the dominant peaks are identical between the three samples.

FIG. 4. Amino acid sequence alignment of PHLPP2 among various species. Site-directed mutagenesis of Ser/Thr to Ala was performed and the phosphorylation of each mutant was tested.

FIG. 5A. Schematic of phos-tag-based mobility shift detection of phosphorylated proteins.

FIG. 5B. Phos-tag-based mobility shift detection of phosphorylated proteins in the absence of phos-tag.

FIG. 5C. Phos-tag-based mobility shift detection of phosphorylated proteins in the presence of 25 μM phos-tag.

FIG. 6. Identification of upstream kinases for PHLPP2 phosphorylation.

FIG. 7A. Identification of phosphorylation sites in PHLPP2. Each identified Ser/Thr site from LC/MS-MS-based phospho-peptide mapping results was individually mutated to Alanine, then transfected WT or Ala-mutant PHLPP2 in hepatocytes. Results in the presence of serum.

FIG. 7B. Identification of phosphorylation sites in PHLPP2. Each identified Ser/Thr site from LC/MS-MS-based phospho-peptide mapping results was individually mutated to Alanine, then transfected WT or Ala-mutant PHLPP2 in hepatocytes. Results in the absence of serum.

FIG. 8A. Forskolin induced phosphbrylation overexpressed PHLPP2 in primary hepatocytes.

FIG. 8B. cAMP activator induces PHLPP2 phosphorylation on two Serine residues. WT, S1119A, and S1210A mutant of PHLPP2 were all phosphorylated with forskolin treatment.

FIG. 8C. cAMP activator induces PHLPP2 phosphorylation on two Serine residues. Ablation of both sites rendered the S1119A/S1210A mutant insensitive to forskolin-induced phosphorylation.

FIG. 9A. Identification of phosphorylation sites in PHLPP2 by Glucagon/PKA signaling. Forskolin-mediated PHLPP2 phosphorylation.

FIG. 9B. Identification of phosphorylation sites in PHLPP2 by Glucagon/PKA signaling. Forskolin-mediated PHLPP2 phosphorylation was reduced by pretreatment of PKA inhibitor, H89.

FIG. 10A. Inhibition of Glucagon signaling increases PHLPP2 protein levels in db/db mice. Glucagon receptor (Gcgr) expression from livers of db/db mice transduced with AAV8-shControl or AAV8-shGcgr.

FIG. 10B. Inhibition of Glucagon signaling increases PHLPP2 protein levels in db/db mice. Western blots from livers of db/db mice transduced with AAV8-shControl or AAV8-shGcgr.

FIG. 10C. Inhibition of Glucagon signaling increases PHLPP2 protein levels in db/db mice. Quantification of Western blots in FIG. 43B, normalized to either total PHLPP2 or β-actin.

FIG. 11A. Inhibition of Glucagon signaling increases PHLPP2 protein levels in DIO mice. Western blots from livers of diet-induced obese (DIO) C57BL6 mice transduced with either AAV8-shControl or AAV8-shGcgr.

FIG. 11B. Inhibition of Glucagon signaling increases PHLPP2 protein levels in DIO mice. Quantification of PHLPP2 levels, normalized to β-actin.

FIG. 12A. Generation of PHLPP2 KO cells using Genome-scale CRISPR Knock-Out (GeCKO) system. PHLPP2 KO HepG2 hepatoma cells were generated using the CRISPR (Clustered Regularly Interspaced Short Palindromic Repeats)-Cas9 system. Using three different single guide RNA (sgRNA) for PHLPP2, significantly reduced PHLPP2 protein and mRNA levels

FIG. 12B. Generation of PHLPP2 KO cells using Genome-scale CRISPR Knock-Out (GeCKO) system. Quantification of PHLPP2 levels in FIG. 45A.

FIG. 12C. Generation of PHLPP2 KO cells using Genome-scale CRISPR Knock-Out (GeCKO) system. Quantifiation of off-target gene expression.

FIG. 13A. Generation of PHLPP2 KO cells using Genome-scale CRISPR Knock-Out (GeCKO) system. HepG2 stable cell lines were generated expressing the most efficient PHLPP2 sgRNA (#3) sgRNA3.

FIG. 13B. Generation of PHLPP2 KO cells using Genome-scale CRISPR Knock-Out (GeCKO) system. Mouse hepatoma cell lines, Hepa1c1c7 cells, were generated.

FIG. 14A. Characterization of PHLPP2 KO cells. PHLPP2-deficient cells showed sustained insulin signaling.

FIG. 14B. Characterization of PHLPP2 KO cells. PHLPP2 levels “rescued” in CRISPR-induced knockout cell lines with either WT or S1119/1210A PHLPP2.

FIG. 15. Identification of novel interaction partners with PHLPP2.

FIG. 16A. Identification of novel interaction partners with PHLPP2-KCTD17 interaction with PHLPP2. PHLPP2 associated with KCTD17 in a proteasome-dependent manner.

FIG. 16B. Identification of novel interaction partners with PHLPP2-KCTD17 interaction with PHLPP2. Association of endogenous PHLPP2 with KCTD17 was significantly enhanced by treatment of forskolin and simultaneous treatment with forskolin and the proteasome inhibitor, MG132.

FIG. 17. Identification of novel interaction partners with PHLPP2-KCTD17 interaction with PHLPP2, phosphorylation-dependent manner.

FIG. 18A. Loss of Phlpp2 causes fatty liver. Hepatic Phlpp2 expression negatively correlates with hepatic lipid across different BXD populations (Spearman rho=−0.60, p=0.04).

FIG. 18B. Loss of Phlpp2 causes fatty liver. Normalized enrichment score (ES) of GSEA indicating gene sets that show the most significant negative correlation with hepatic Phlpp2 expression.

FIG. 18C. Loss of Phlpp2 causes fatty liver. Heat map showing co-regulation of Phlpp2 and lipogenic genes in BXD strains.

FIG. 18D. Loss of Phlpp2 causes fatty liver. Liver weight in Cre-control and liver-specific PHLPP2 knockout (L-PHLPP2) mice fasted for 18 h and then refed for 8 h (n=9-10/group).

FIG. 18E. Loss of Phlpp2 causes fatty liver. Triglycerides Ln Cre-control and liver-specific PHLPP2 knockout (L-PHLPP2) mice fasted for 18 h and then refed for 8 h (n=9-10/group).

FIG. 18F. Loss of Phlpp2 causes fatty liver. Western blots of liver lysates in Cre-control and liver-specific PHLPP2 knockout (L-PHLPP2) mice fasted for 18 h and then refed for 8 h (n=9-10/group)

FIG. 18G. Loss of Phlpp2 causes fatty liver. Western blots of hepatic gene expression in Cre-control and liver-specific PHLPP2 knockout (L-PHLPP2) mice fasted for 18 h and then refed for 8 h (n=9-10/group).

FIG. 18H. Loss of Phlpp2 causes fatty liver. Western blots from control, PHLPP2 KO, or PHLPP2-reconstituted PHLPP2 KO cells “pulsed” with 10 nM insulin for 30 min, with or without a 2 h “chase” in insulin-free medium. *P<0.05, **P<0.01, ***P<0.001 as compared to the indicated control by two-way ANOVA. All data are shown as the means±s.e.m.

FIG. 19A. Phlpp2 expression is inversely correlated with lipogenic gene expression. Geneset enrichment analysis (GSEA) using all hepatic transcriptomes (GSE60149) of B×D lines, highlighting the strains showing lowest (left) or highest (right) Phlpp2 expression.

FIG. 19B. Phlpp2 expression is inversely correlated with lipogenic gene expression. Normalized enrichment score of GSEA indicating genesets that show negative correlation with hepatic Phlpp2 expression.

FIG. 20A. Generation of conditional PHLPP2 knockout mice. Targeting strategy to generate PHLPP2 flox/flox allele.

FIG. 20B. Generation of conditional PHLPP2 knockout mice. Genotyping analysis of successfully target allele.

FIG. 20C. Generation of conditional PHLPP2 knockout mice. Western blots from liver, epididymal (eWAT) oringuinal white adipose tissue (iWAT), brown adipose tissue (BAT), and whole pancreas in PHLPP2 floxed mice transduced with AAV8-TBG-GFP or AAV8-TBG-Cre.

FIG. 20D. Generation of conditional PHLPP2 knockout mice. Western blots inisolated primary hepatocytes from PHLPP2 flox/flox mice, transduced with Ad-GFP or Ad-Cre.

FIG. 21A. Prolonged Akt phosphorylation in L-PHLPP2 mice leads to fatty liver. Body weight in Cre- or L-PHLPP2 mice, sacrificed after a 18 h fast followed by 8 h refeeding (n=9-10/group).

FIG. 21B. Prolonged Akt phosphorylation in L-PHLPP2 mice leads to fatty liver. Normalized epidydimal (eWAT) oringuinal (iWAT) white adipose tissue weight in Cre- or L-PHLPP2 mice, sacrificed after a 18 h fast followed by 8 h refeeding (n=9-10/group).

FIG. 21C. Prolonged Akt phosphorylation in L-PHLPP2 mice leads to fatty liver. Liver cholesterol in Cre- or L-PHLPP2 mice, sacrificed after a 18 h fast followed by 8 h refeeding (n=9-10/group).

FIG. 21D. Prolonged Akt phosphorylation in L-PHLPP2 mice leads to fatty liver. Plasma cholesterol in Cre- or L-PHLPP2 mice, sacrificed after a 18 h fast followed by 8 h refeeding (n=9-10/group).

FIG. 21E. Prolonged Akt phosphorylation in L-PHLPP2 mice leads to fatty liver. Non-esterified fatty acid (NEFA) in Cre- or L-PHLPP2 mice, sacrificed after a 18 h fast followed by 8 h refeeding (n=9-10/group).

FIG. 21F. Prolonged Akt phosphorylation in L-PHLPP2 mice leads to fatty liver. Plasma triglyceride in Cre- or L-PHLPP2 mice, sacrificed after a 18 h fast followed by 8 h refeeding (n=9-10/group).

FIG. 69G. Prolonged Akt phosphorylation in L-PHLPP2 mice leads to fatty liver. Western blots from primary hepatocyte sisolated from PHLPP2 flox/flox mice, transduced with Ad-GFP or Ad-Cre, then exposed to medium with or without 10 nM insulin for 6 h.

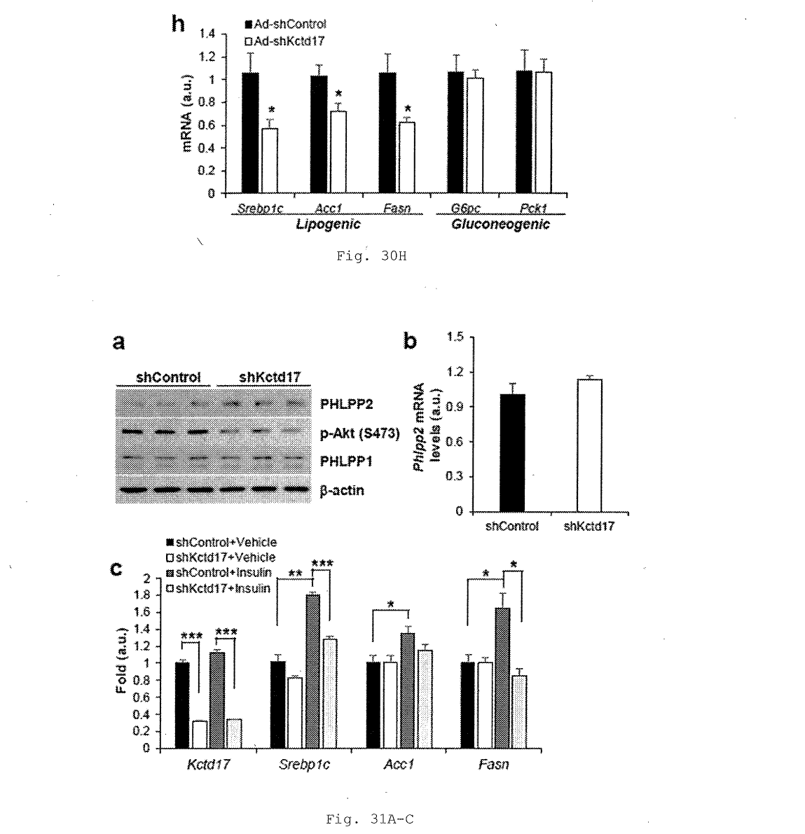

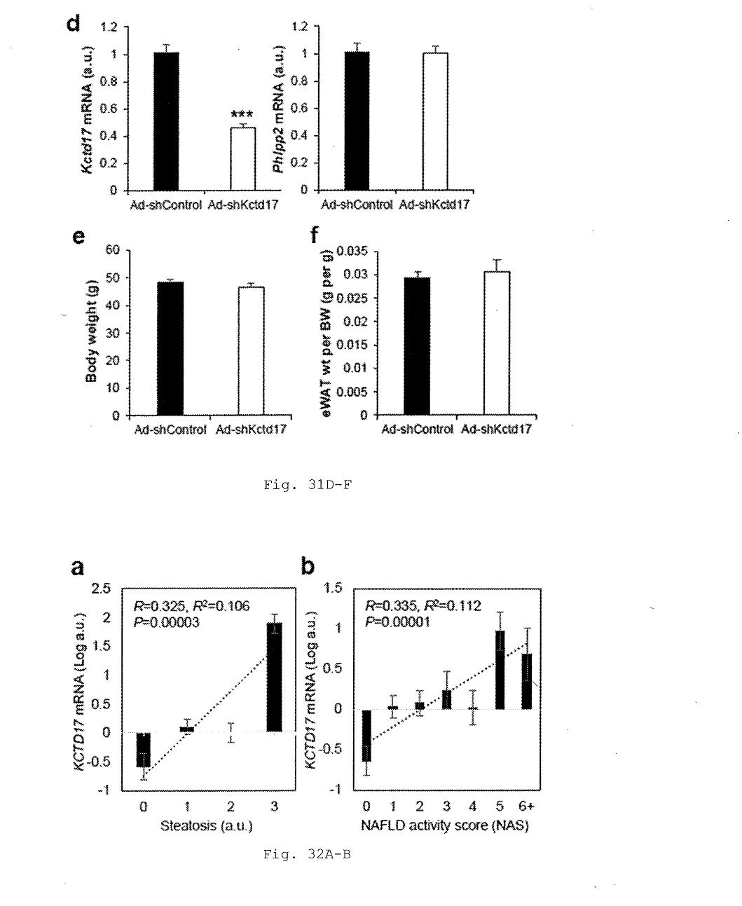

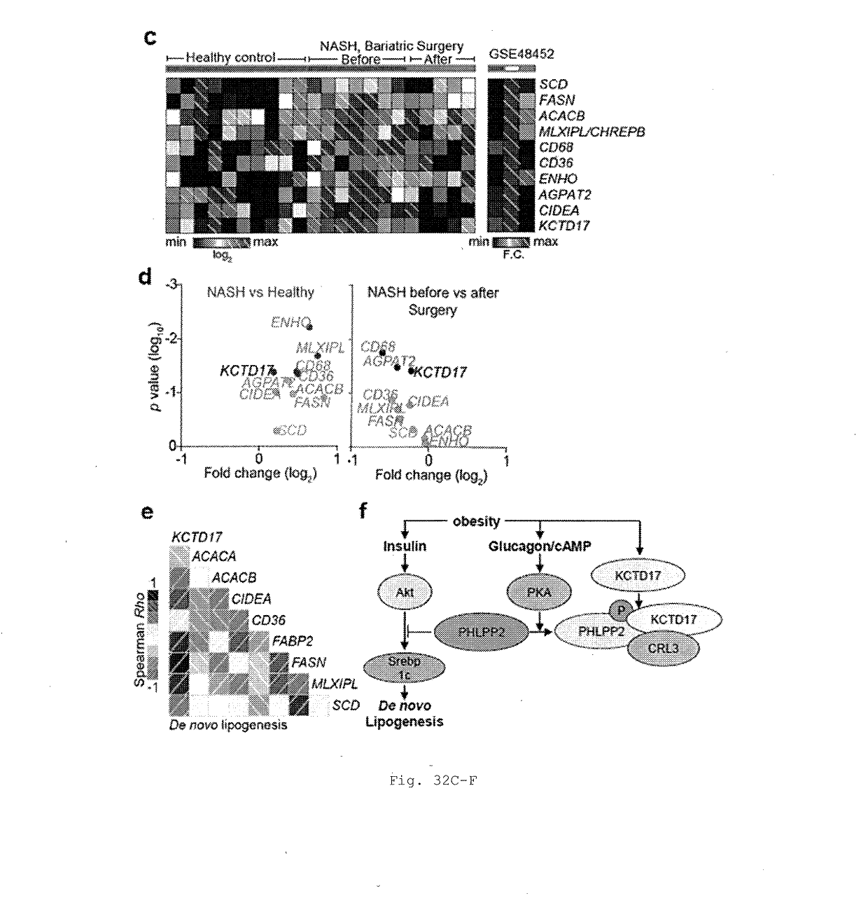

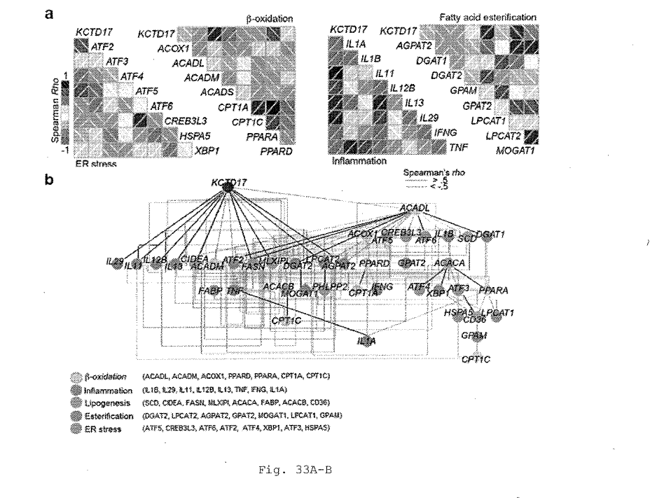

FIG. 21H. Prolonged Akt phosphorylation in L-PHLPP2 mice leads to fatty liver. Blood glucose in Cre- and L-PHLPP2 mice. *P<0.05 as compared to the indicated control by two-way ANOVA. All data are shown as the means±s.e.m.

FIG. 21I. Prolonged Akt phosphorylation in L-PHLPP2 mice leads to fatty liver. Insulin in Cre- and L-PHLPP2 mice. *P<0.05 as compared to the indicated control by two-way ANOVA. All data are shown as the means±s.e.m.

FIG. 21J. Prolonged Akt phosphorylation in L-PHLPP2 mice leads to fatty liver. Intraperitoneal glucose tolerance test (GTT) in Cre- and L-PHLPP2 mice. *P<0.05 as compared to the indicated control by two-way ANOVA. All data are shown as the means±s.e.m.

FIG. 22A. HFD-fed L-PHLPP2 mice show normal glucose/lipid homeostasis. Liver weight in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22B. HFD-fed L-PHLPP2 mice show normal glucose/lipid homeostasis. Triglyceride in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22C. HFD-fed L-PHLPP2 mice show normal glucose/lipid homeostasis. Body weight in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22D. HFD-fed L-PHLPP2 mice show normal glucose/lipid homeostasis. Blood glucose in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22E. HFD-fed L-PHLPP2 mice show normal glucose/lipid homeostasis. eWAT weight in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22F. HFD-fed L-PHLPP2 mice show normal glucose/lipid homeostasis. Liver protein in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22G. HFD-fed L-PHLPP2 mice show normal glucose/lipid homeostasis. Plasma triglyceride in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22H. HFD-fed L-PHLPP2 mice show normal glucose/lipid homeostasis. Cholesterol in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22I. HFD-fed L-PHLPP2 mice show normal glucose/lipid homeostasis. NEFA levels in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 22J. HFD-fed L-PHLPP2 mice show normal glucose/lipid homeostasis. Liver mRNA in HFD-fed Cre- or L-PHLPP2 mice, sacrificed after a 18 h fast followed by 8 h refeeding. (n=9-10/group). ***P<0.001 as compared to Cre-mice by two-way ANOVA. All data are shown as the means±s.e.m.

FIG. 23A. PHLPP2KO cells have prolonged insulin-mediated lipogenic gene expression. Generation of PHLPP2 KO cells using CRISPR/Cas9 by transducing HepG2 hepatoma cells with lentivirus expressing three different single guide RNA (sgRNA

FIG. 23B. PHLPP2KO cells have prolonged insulin-mediated lipogenic gene expression. Generation of PHLPP2 KO cells using CRISPR/Cas9 by transducing HepG2 hepatoma cells with lentivirus expressing three different single guide RNA (sgRNA).

FIG. 23C. PHLPP2KO cells have prolonged insulin-mediated lipogenic gene expression. qPCR analysis of predicted potential off-target genes for sgRNAs. *P<0.05 and **P<0.01 as compared to the indicated control by two-way ANOVA. All data are shown as the means±s.e.m.

FIG. 23D. PHLPP2KO cells have prolonged insulin-mediated lipogenic gene expression. qPCR analysis in control and PHLPP2 KO cells after insul in exposure for 6 h. *P<0.05 and **P<0.01 as compared to the indicated control by two-way ANOVA. All data are shown as the means±s.e.m.

FIG. 24A. PHLPP2 is phosphorylated on Ser1119 and Ser1210 by glucagon/PKA action. Measured and theoretical mass (singly charged) of phosphopeptides of PHLPP2 isolated from Ad-PHLPP-transduced liver via immunoprecipitation and analyzed by LC-MS/MS, with the phosphorylated serine residue indicated with

FIG. 24B. PHLPP2 is phosphorylated on Ser1119 and Ser1210 by glucagon/PKA action. PHLPP2 contains Ras-association (RA), pleckstrin homology (PH), hydrophobic leucine-rich repeat (LRR), protein phosphatase 2C (PP2C) domains; amino acid sequence comparison from selected mammalian species with PKA targeting sequence (red) and phosphorylated Ser (blue) residues highlighted.

FIG. 24C. PHLPP2 is phosphorylated on Ser1119 and Ser1210 by glucagon/PKA action. Western blots from primary hepatocytes exposed to 100 nM glucagon, using Phos-tag to separate phosphorylated from unphosphorylated PHLPP2.

FIG. 24D. PHLPP2 is phosphorylated on Ser1119 and Ser1210 by glucagon/PKA action. Western blots from primary hepatocytes exposed to 100 μM cAMP or 10 □M forskolin for 1 h, using Phos-tag to separate phosphorylated from unphosphorylated PHLPP2.

FIG. 24E. PHLPP2 is phosphorylated on Ser1119 and Ser1210 by glucagon/PKA action. Western blots from primary hepatocytes exposed to glucagon for the indicated times with or without forskolin.

FIG. 24F. PHLPP2 is phosphorylated on Ser1119 and Ser1210 by glucagon/PKA action. Western blots from HepG2 cells stably expressing two different shRNA directed at PKA or shControl (−) with or without forskolin.

FIG. 24G. PHLPP2 is phosphorylated on Ser1119 and Ser1210 by glucagon/PKA action. Western blots from primary hepatocytes exposed to Co-immunoprecipitation (CoIP) of PHLPP2 (WT) but not nonphosphorylatable mutant PHLPP2 (2A) with phospho-PKA substrate in response to forskolin, sensitive to the PKA inhibitor H-89, in primary hepatocytes.

FIG. 25A. Glucagon/PKA-induced phosphorylation of PHLPP2 at Ser1119 and Ser1210. Primary hepatocytes were incubated with insulin or the indicated kinase inhibitors prior to Phos-tag Western blot.

FIG. 25B. Glucagon/PKA-induced phosphorylation of PHLPP2 at Ser1119 and Ser1210. Transfected with PHLPP2-WT, S1119A, S1210A with or without forskolin, prior to Phos-tag Western blot.

FIG. 25C. Glucagon/PKA-induced phosphorylation of PHLPP2 at Ser1119 and Ser1210. Validation of novel PHLPP2 Ser1119- or 1210-specific antibodies in hepatocytes transfected with empty vector (EV), PHLPP2 (WT), or nonphosphorylatable mutant PHLPP2 (2A) with or without forskolin.

FIG. 25D. Glucagon/PKA-induced phosphorylation of PHLPP2 at Ser1119 and Ser1210. Validation of novel PHLPP2 Ser1119- or 1210-specific antibodies in hepatocytes transfected with EV, WT, S1119A, S1210A, or 2A.

FIG. 25E. Glucagon/PKA-induced phosphorylation of PHLPP2 at Ser1119 and Ser1210. CoIP of WT but not 2A with phospho-PKA substrate antibody in response to glucagon or forskolin in primary hepatocytes.

FIG. 26A. Glucagon/PKA-induced phosphorylation of PHLPP2 at Ser1119 and Ser1210 in vivo. Western blots of liver lysate of p-PHLPP2/total PHLPP2 and PHLPP2/b-actin. *P<0.05 and **P<0.01 as compared to fasted mice by two-way ANOVA. All data are shown as the means±s.e.m.

FIG. 26B. Glucagon/PKA-induced phosphorylation of PHLPP2 at Ser1119 and Ser1210 in vivo. Quantification of p-PHLPP2/total PHLPP2 and PHLPP2/b-actin. *P<0.05 and **P<0.01 as compared to fasted mice by two-way ANOVA. All data are shown as the means±s.e.m.

FIG. 27A. Glucagon-mediated PHLPP2 phosphorylation and destabilization in obesity. Western blots of liver lysate of p-PHLPP2, total PHLPP2 and β-actin, and corresponding liver Phlpp2 expression in C57BL/6 wild-type mice fed normal chow or HFD for 16 weeks.

FIG. 27B. Glucagon-mediated PHLPP2 phosphorylation and destabilization in obesity. Western blot quantification of p-PHLPP2/total PHLPP2 and PHLPP2/β-actin in C57BL/6 wild-type mice fed normal chow or HFD for 16 weeks.

FIG. 27C. Glucagon-mediated PHLPP2 phosphorylation and destabilization in obesity. Western blot quantification of corresponding liver Phlpp2 expression in C57BL/6 wild-type mice fed normal chow or HFD for 16 weeks.

FIG. 27D. Glucagon-mediated PHLPP2 phosphorylation and destabilization in obesity. Western blots of liver lysate of p-PHLPP2, total PHLPP2 and β-actin, and corresponding liver Phlpp2 expression 8-week-old C57BL/6J wild-type as compared to db/db mice.

FIG. 27E. Glucagon-mediated PHLPP2 phosphorylation and destabilization in obesity. Western blot quantification of p-PHLPP2/total PHLPP2 and PHLPP2/β-actin in 8-week-old C57BL/6J wild-type as compared to db/db mice.

FIG. 27F. Glucagon-mediated PHLPP2 phosphorylation and destabilization in obesity. Western blot quantification of corresponding liver Phlpp2 expression in C57BL/6 wild-type mice fed normal chow or HFD for 16 weeks.

FIG. 27G. Glucagon-mediated PHLPP2 phosphorylation and destabilization in obesity. Western blot of PHLPP2 in cycloheximide (CHX, 50 mg m1⁻¹)-treated primary hepatocytes, with or without forskolin, with quantification of PHLPP2/α-tubulin relative to time 0.

FIG. 27H. Glucagon-mediated PHLPP2 phosphorylation and destabilization in obesity. qPCR from AAV8-shGcgr-transduced, HFD-fed C57BL/6 and db/db mice. *P<0.05, **P<0.01, ***P<0.001 as compared to the indicated control by two-way ANOVA. All data are shown as the means±s.e.m.

FIG. 27I. Glucagon-mediated PHLPP2 phosphorylation and destabilization in obesity. Western blots from AAV8-shGcgr-transduced, HFD-fed C57BL/6 and db/db mice. *P<0.05, **P<0.01, ***P<0.001 as compared to the indicated control by two-way ANOVA. All data are shown as the means±s.e.m.

FIG. 28A. KCTD17 binds phosphorylated PHLPP2 to induce its degradation in obese liver. Liver Kctd17 expression in C57BL/6 wild-type mice fed normal chow vs. HFD for either 2 or 4 months or C57BL/6J wild-type vs. db/db mice.

FIG. 28B. KCTD17 binds phosphorylated PHLPP2 to induce its degradation in obese liver. Endogenous CoIP of KCTD17 and PHLPP2 in liver lysate from HFD-fed mice, fasted for 18 h and then refed for 8 h.

FIG. 28C. KCTD17 binds phosphorylated PHLPP2 to induce its degradation in obese liver. CoIP of KCTD17 by PHLPP2 in primary hepatocytes is increased by MG-132

FIG. 28D. KCTD17 binds phosphorylated PHLPP2 to induce its degradation in obese liver. CoIP of KCTD17 by PHLPP2 in primary hepatocytes is increased synergistically by forskolin.

FIG. 28E. KCTD17 binds phosphorylated PHLPP2 to induce its degradation in obese liver. KCTD17 facilitates the interaction of PHLPP2 (WT), with the E3-ligase, Cullin3.

FIG. 28F. KCTD17 binds phosphorylated PHLPP2 to induce its degradation in obese liver. KCTD17 does not facilitate the interaction of the nonphosphorylatable mutant PHLPp2 (2A), with the E3-ligase, Cullin3.

FIG. 28G. KCTD17 binds phosphorylated PHLPP2 to induce its degradation in obese liver. Western blots of liver lysate from HFD-fed C57BL/6 mice transduced with Ad-GFP, Ad-PHLPP2 (WT), or Ad-PHLPP2 (2A), fasted for 18 h and then refed for 8 h prior to sacrifice.

FIG. 28H. KCTD17 binds phosphorylated PHLPP2 to induce its degradation in obese liver. Liver weight from HFD-fed C57BL/6 mice transduced with Ad-GFP, Ad-PHLPP2 (WT), or Ad-PHLPP2 (2A), fasted for 18 h and then refed for 8 h prior to sacrifice. (n=6-7/group). *P<0.05, **P<0.01 as compared to the indicated control by two-way ANOVA. All data are shown as the means±s.e.m.

FIG. 28I. KCTD17 binds phosphorylated PHLPP2 to induce its degradation in obese liver. Triglycerides from HFD-fed C57BL/6 mice transduced with Ad-GFP, Ad-PHLPP2 (WT), or Ad-PHLPP2 (2A), fasted for 18 h and then refed for 8 h prior to sacrifice. (n=6-7/group). *P<0.05, **P<0.01 as compared to the indicated control by two-way ANOVA. All data are shown as the means±s.e.m.

FIG. 28J. KCTD17 binds phosphorylated PHLPP2 to induce its degradation in obese liver. Plasma triglycerides and (k) hepatic gene expression from HFD-fed C57BL/6 mice transduced with Ad-GFP, Ad-PHLPP2 (WT), or Ad-PHLPP2 (2A), fasted for 18 h and then refed for 8 h prior to sacrifice. (n=6-7/group). *P<0.05, **P<0.01 as compared to the indicated control by two-way ANOVA. All data are shown as the means±s.e.m.

FIG. 28K. KCTD17 binds phosphorylated PHLPP2 to induce its degradation in obese liver. Hepatic gene expression from HFD-fed C57BL/6 mice transduced with Ad-GFP, Ad-PHLPP2 (WT), or Ad-PHLPP2 (2A), fasted for 18 h and then refed for 8 h prior to sacrifice. (n=6-7/group). *P<0.05, **P<0.01 as compared to the indicated control by two-way ANOVA. All data are shown as the means±s.e.m.

FIG. 29A. Similar expression of WT and 2A in obese mice. Hepatic Phlpp2 expression in HFD-fed C57BL/6 mice transduced with Ad-GFP, Ad-PHLPP2 (WT), or Ad-PHLPP2(2A), fasted for 18 h and then refed for 8 h prior to sacrifice. (n=6-7/group). ***P<0.001 as compared to Ad-GFP-transduced mice by two-way ANOVA. All data are shown as the means±s.e.m.

FIG. 29B. Similar expression of WT and 2A in obese mice. Body weight in HFD-fed C57BL/6 mice transduced with Ad-GFP, Ad-PHLPP2 (WT), or Ad-PHLPP2(2A), fasted for 18 h and then refed for 8 h prior to sacrifice. (n=6-7/group). ***P<0.001 as compared to Ad-GFP-transduced mice by two-way ANOVA. All data are shown as the means±s.e.m.

FIG. 29C. Similar expression of WT and 2A in obese mice. Normalized eWAT weight in HFD-fed C57BL/6 mice transduced with Ad-GFP, Ad-PHLPP2 (WT), or Ad-PHLPP2(2A), fasted for 18 h and then refed for 8 h prior to sacrifice. (n=6-7/group). ***P<0.001 as compared to Ad-GFP-transduced mice by two-way ANOVA. All data are shown as the means±s.e.m.

FIG. 29D. Similar expression of WT and 2A in obese mice. Blood glucose levels in HFD-fed C57BL/6 mice transduced with Ad-GFP, Ad-PHLPP2 (WT), or Ad-PHLPP2(2A), fasted for 18 h and then refed for 8 h prior to sacrifice. (n=6-7/group). ***P<0.001 as compared to Ad-GFP-transduced mice by two-way ANOVA. All data are shown as the means±s.e.m.

FIG. 29E. Similar expression of WT and 2A in obese mice. Insulin levels in HFD-fed C57BL/6 mice transduced with Ad-GFP, Ad-PHLPP2 (WS), or Ad-PHLPP2(2A), fasted for 18 h and then refed for 8 h prior to sacrifice. (n=6-7/group). ***P<0.001 as compared to Ad-GFP-transduced mice by two-way ANOVA. All data are shown as the means±s.e.m.

FIG. 30A. Knockdown of hepatic KCTD17 prevents diet-induced hepatic steatosis. qPCR from primary hepatocytes transduced with Ad-shKctd17.

FIG. 30B. Knockdown of hepatic KCTD17 prevents diet-induced hepatic steatosis. Western blots from primary hepatocytes transduced with Ad-shKctd17.

FIG. 30C. Knockdown of hepatic KCTD17 prevents diet-induced hepatic steatosis. Knockdown of Kctd17 reduces endogenous PHLPP2 ubiquitination in HA/Ub-transfected primary hepatocytes.

FIG. 308D. Knockdown of hepatic KCTD17 prevents diet-induced hepatic steatosis. Western blots from liver lysate in HFD-fed C57BL/6 mice transduced with Ad-shControl or Ad-shKctd17, fasted for 18 h and then refed for 8 h prior to sacrifice (n=6-7/group). *P<0.05, **P<0.01, ***P<0.001 as compared to the indicated control by two-way ANOVA. All data are shown as the means±s.e.m.

FIG. 30E. Knockdown of hepatic KCTD17 prevents diet-induced hepatic steatosis. Liver weight in HFD-fed C57BL/6 mice transduced with Ad-shControl or Ad-shKctd17, fasted for 18 h and then refed for 8 h prior to sacrifice (n=6-7/group). *P<0.05, **P<0.01, ***P<0.001 as compared to the indicated control by two-way ANOVA. All data are shown as the means±s.e.m.

FIG. 30F. Knockdown of hepatic KCTD17 prevents diet-induced hepatic steatosis. Triglycerides in HFD-fed C57BL/6 mice transduced with Ad-shControl or Ad-shKctd17, fasted for 18 h and then refed for 8 h prior to sacrifice (n=6-7/group). *P<0.05, **P<0.01, ***P<0.001 as compared to the indicated control by two-way ANOVA. All data are shown as the means±s.e.m.

FIG. 30G. Knockdown of hepatic KCTD17 prevents diet-induced hepatic steatosis. Liver H&E and Oil-Red-O staining in HFD-fed C57BL/6 mice transduced with Ad-shControl or Ad-shKctd17, fasted for 18 h and then refed for 8 h prior to sacrifice (n=6-7/group). *P<0.05, **P<0.01, ***P<0.001 as compared to the indicated control by two-way ANOVA. All data are shown as the means±s.e.m.

FIG. 30H. Knockdown of hepatic KCTD17 prevents diet-induced hepatic steatosis. Hepatic gene expression in HFD-fed C57BL/6 mice transduced with Ad-shControl or Ad-shKctd17, fasted for 18 h and then refed for 8 h prior to sacrifice (n=6-7/group). *P<0.05, **P<0.01, ***P<0.001 as compared to the indicated control by two-way ANOVA. All data are shown as the means±s.e.m.

FIG. 31A. Knockdown of KCTD17 prevents hepatic steatosis. Western blots from Hepa1c1c7 cells transduced with Ad-shControl or Ad-shKctd17.

FIG. 31B. Knockdown of KCTD17 prevents hepatic steatosis. qPCR from Hepa1c1c7 cells transduced with Ad-shControl or Ad-shKctd17.

FIG. 31C. Knockdown of KCTD17 prevents hepatic steatosis. Lipogenic gene expression in primary hepatocytes transduced with Ad-shControl or Ad-shKctd17 after insulin exposure for 6 h (n=4 biologic replicates).

FIG. 31D. Knockdown of KCTD17 prevents hepatic steatosis. qPCR analysis in HFD-fed C57BL/6 mice transduced with Ad-shControl or Ad-shKctd17, fasted for 18 h and then refed for 8 h prior to sacrifice (n=6-7/group). *P<0.05, **P<0.01, and ***P<0.001 as compared to the indicated control by two-way ANOVA. All data are shown as the means±s.e.m.

FIG. 31E. Knockdown of KCTD17 prevents hepatic steatosis. Body weight in HFD-fed C57BL/6 mice transduced with Ad-shControl or Ad-shKctd17, fasted for 18 h and then refed for 8 h prior to sacrifice (n=6-7/group). *P<0.05, **P<0.01, and ***P<0.001 as compared to the indicated control by two-way ANOVA. All data are shown as the means±s.e.m.

FIG. 32A. KCTD17 correlates with lipogenic gene expression and hepatic steatosis in NASH. KCTD17 expression in liver biopsy specimens (n=158) as related to steatosis subscore.

FIG. 32B. KCTD17 correlates with lipogenic gene expression and hepatic steatosis in NASH. KCTD17 expression in liver biopsy specimens (n=158) as related to total NAFLD activity score (NAS).

FIG. 32C. KCTD17 correlates with lipogenic gene expression and hepatic steatosis in NASH. Heat map of hepatic KCTD17 expression and de novo lipogenesis (DNL) gene set in hepatic transcriptomes of healthy controls and NASH patients before and after bariatric surgery (n=5-10/group), with the depth of shading of correlogram according to the magnitude of the correlation and positive and negative correlations.

FIG. 32D. KCTD17 correlates with lipogenic gene expression and hepatic steatosis in NASH. Volcano plot of hepatic KCTD17 expression and de novo lipogenesis (DNL) gene set in hepatic transcriptomes of healthy controls and NASH patients before and after bariatric surgery (n=5-10/group), with the depth of shading of correlogram according to the magnitude of the correlation and positive and negative correlations represented in blue and red, respectively.

FIG. 32E. KCTD17 correlates with lipogenic gene expression and hepatic steatosis in NASH. Correlogram of hepatic KCTD17 expression and de novo lipogenesis (DNL) gene set in hepatic transcriptomes of healthy controls and NASH patients before and after bariatric surgery (n=5-10/group), with the depth of shading of correlogram according to the magnitude of the correlation and positive and negative correlations.

FIG. 32F. KCTD17 correlates with lipogenic gene expression and hepatic steatosis in NASH. Model representing the parallel effects of obesity to increase glucagon-mediated PHLPP2 phosphorylation and KCTD17 expression/activity, which synergistically induce PHLPP2 degradation and prolongation of Akt-induced Srebp1c-mediated DNL, and fatty liver.

FIG. 33A. KCTD17 expression is correlated with multiple potentially pathogenic pathways in patients with NASH. Correlogram indicating Spearman's correlation between KCTD17 and gene sets of indicated pathways from NASH subjects.

FIG. 33B. KCTD17 expression is correlated with multiple potentially pathogenic pathways in patients with NASH. Interaction network showing KCTD17 expression association with genes sets involved in DNL, fatty acid esterification, fatty acid oxidation, ER stress, and inflammation, with black and grey connecting lines indicating positive and negative correlations, respectively. Only correlations with Spearman's Rho >0.5 or <−0.5 are shown.

### DETAILED DESCRIPTION OF THE INVENTION

### Embodiments of the Invention

The present invention provides a method of reducing a subject's hepatic and plasma triglyceride levels comprising administering to a subject in need thereof a pharmaceutical composition comprising a pharmaceutical carrier and a compound that decreases KCTD17 expression in liver cells in an amount effective to reduce the subject's hepatic and plasma triglyceride levels.

The present invention provides a method of reducing a subject's hepatic and plasma triglyceride levels comprising administering to a subject in need thereof a pharmaceutical composition comprising a pharmaceutical carrier and a compound that increases PHLPP2 in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that increases free Raptor in liver cells in an amount effective to reduce the subject's hepatic and plasma triglyceride levels.

In some embodiments, the pharmaceutical composition increases Raptor expression, thereby increasing free Raptor in the liver cells.

In some embodiments, the pharmaceutical composition inhibits interaction of Raptor and mTORC1, thereby increasing free Raptor in the liver cells.

In some embodiments, the compound reduces the expression of at least one lipogenic gene.

In some embodiments, the at least one lipogenic gene is Srebp1c, Fasn, Acc1, or Scd1.

In some embodiments, the subject is afflicted with a metabolic disease.

In some embodiments, the pharmaceutical composition comprises a polynucleotide.

In some embodiments, the pharmaceutical composition is targeted to the liver of the subject.

In some embodiments, the metabolic disease is obesity.

In some embodiments, the metabolic disease is hypertriglyceridemia.

In some embodiments, the metabolic disease is hyperinsulinemia.

In some embodiments, the metabolic disease is Type 2 Diabetes.

In some embodiments, the metabolic disease is fatty liver disease.

In some embodiments, the fatty liver disease is nonalcoholic fatty liver disease or nonalcholic steatohepatitis.

In some embodiments, the subject is afflicted with cirrhosis or hepatocellular carcinoma.

In some embodiments, the subject is a human.

In some embodiments, the subject's hepatic or plasma triglyceride levels are >150 mg/dL.

In some embodiments, the subject's hepatic or plasma triglyceride levels are >500 mg/dL, about 200 to 499 mg/dL, or about 150 to 199 mg/dL.

In some embodiments, the subject's hepatic or plasma triglyceride levels are reduced by at least 20%, at least 25%, at least 30%, at least 35%, at least 40%, at least 45%, at least 50%, at least 55%, at least 60%, at least 65%, at least 70%, or at least 75%, relative to the level prior to the administration.

The present invention also provides a process for determining the amount of free Raptor in a subject's liver comprising:
d) obtaining a biological sample comprising liver cells of the subject;
e) separating free Raptor and mTORC1-associated Raptor in the sample; and
f) determining the amount of free Raptor in the sample.

The present invention also provides a process for diagnosing whether
a subject is afflicted with decreased free Raptor comprising:
a) determining the amount of free Raptor in the subject according to the process of the claimed invention;
b) determining the amount of free Raptor in a reference subject according to the process of the claimed invention; and
d) diagnosing the subject to be afflicted with decreased free Raptor if the amount of free Raptor in step (a) is substantially decreased compared to the amount of free Raptor in step (b).

The present invention also provides a method of treating a subject diagnosed to be afflicted with decreased free Raptor according to the process of the claimed invention comprising reducing the subject's hepatic and plasma triglyceride levels according to the method of the claimed invention.

The present invention also provides a process for determining the amount of KCTD17 expression in a subject's liver comprising:

a) obtaining a biological sample comprising liver cells of the subject;
b) determining the amount of KCTD17 mRNA in the sample.

The present invention also provides a process for diagnosing whether a subject is afflicted with increased KCTD17 expression comprising:

a) determining the amount of KCTD17 in the subject according to the process of claim 31;
b) determining the amount of KCTD17 in a reference subject according to the process of claim 31; and
c) diagnosing the subject to be afflicted with increased KCTD17 expression if the amount of KCTD17 expression in step (a) is substantially increased compared to the amount of KCTD17 expression in step (b).

The present invention also provides a method of treating a subject diagnosed to be afflicted with increased KCTD17 expression according to the process of claim 32 comprising reducing the subject's hepatic and plasma triglyceride levels according to a method of the present invention.

The present invention also provides a method of treating a subject afflicted with elevated triglyceride levels comprising administering to the subject a pharmaceutical composition comprising a pharmaceutical carrier and a compound that decreases KCTD17 expression in liver cells in an amount effective to reduce the subject's hepatic and plasma triglyceride levels.

The present invention also provides a method of treating a subject afflicted with elevated triglyceride levels comprising administering to the subject a pharmaceutical composition comprising a pharmaceutical carrier and a compound that decreases PHLPP2 phosphorylation at Serine 1119 and Serine 1210 residues in liver cells in an amount effective to reduce the subject's hepatic and plasma triglyceride levels.

The present invention also provides a method of treating a subject at risk of developing elevated triglyceride levels comprising administering to the subject a pharmaceutical composition comprising a pharmaceutical carrier and a compound that decreases KCTD17 expression in liver cells in an amount effective to reduce the subject's hepatic and plasma triglyceride levels.

The present invention also provides a method of treating a subject at risk of developing elevated triglyceride levels comprising administering to the subject a pharmaceutical composition comprising a pharmaceutical carrier and a compound that increases PHLPP2 phosphorylation at Serine 1119 and Serine 1210 residues in liver cells in an amount effective to reduce the subject's hepatic and plasma triglyceride levels.

In some embodiments, the pharmaceutical composition reduces β-TrCP-mediated degradation of PHLPP2, thereby increasing free Raptor in the liver cells.

In some embodiments, the pharmaceutical composition comprises a Notch antagonist.

In some embodiments, the Notch antagonist comprises a γ-secretase inhibitor, anti-DLL4 mAB, or a DLK peptide.

In some embodiments, the pharmaceutical composition decreases PHLPP2 phosphorylation at Serine 1119 or Serine 1210 residues.

In some embodiments, the pharmaceutical composition inhibits interaction of PHLPP2 and KCTD17.

In some embodiments, the pharmaceutical composition decreases Akt signaling.

In some embodiments, the pharmaceutical composition decreases Akt phosphorylation at Serine 473 residue.

In some embodiments, the pharmaceutical composition prevents PHLPP2 degradation by inhibiting Glucagon signaling.

In some embodiments, the pharmaceutical composition reduces PHLPP2 degradation, thereby increasing free Raptor in the liver cells.

In some embodiments, the pharmaceutical composition prevents PHLPP2 degradation, thereby increasing free Raptor in the liver cells.

### Methods of Inhibiting Glucagon Signaling

As used herein, “inhibiting glucagon signaling” includes reducing glucagon receptor expression, reducing glucagon receptor activation, blocking glucagon receptor activation, reducing endogenous glucagon action, or blocking glucagon action.

As used herein, “prevents PHLPP2 degradation” includes inhibiting PHLPP2 phosphorylation, thereby increasing PHLPP2 levels compared to controls.

As used herein, “reduces PHLPP2 degradation” includes inhibiting PHLPP2 phosphorylation, thereby increasing PHLPP2 levels compared to controls.

In some embodiments, the pharmaceutical composition inhibits Glucagon signaling, thereby reducing PHLPP2 degradation.

In some embodiments, the pharmaceutical composition comprises a Glucagon receptor (Gcgr) antagonizing antibody.

In some embodiments, the pharmaceutical composition comprises a Glucagon receptor (Gcgr) antagonist.

In some embodiments, the Glucagon receptor antagonist reduces PHLPP2 degradation.

In some embodiments, the Glucagon receptor antagonist is des-His¹-[Glu⁹]-Glucagon (1-29) amide, or L-168,049.

In some embodiments, the Glucagon receptor antagonist is 4-[3-(5-Bromo-2-propoxyphenyl)-5-(4-chlorophenyl)-1H-pyrrol-2-yl]pyridine.

In some embodiments, glucagon is suppressed by somatostatin infusion.

The present invention also provides a method of reducing a subject's hepatic and plasma triglyceride levels comprising administering to a subject in need thereof a pharmaceutical composition comprising a pharmaceutical carrier and a compound that prevents PHLPP2 degradation in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that inhibits Glucagon signaling in liver cells in an amount effective to reduce the subject's hepatic and plasma triglyceride levels.

In some embodiments, the pharmaceutical composition prevents PHLPP2 degradation, thereby increasing free Raptor in the liver cells.

In some embodiments, each compound administered to the subject is, independently, an organic compound having a molecular weight less than 1000 Daltons, a DNA aptamer, an RNA aptamer, a polypeptide, an antibody, an oligonucleotide, an interfering RNA (RNAi) molecule, a ribozyme, or a small molecule inhibitor.

In some embodiments, a compound that is capable of inhibiting glucagon signaling is administered to the subject.

In some embodiments, the compound which is capable of inhibiting glucagon signaling is an organic compound having a molecular weight less than 1000 Daltons.

In some embodiments, the oligonucleotide is an antisense oligonucleotide, an RNA-interference inducing compound, or a ribozyme.

In some embodiments, the oligonucleotide is targeted to hepatocytes.

In some embodiments, the oligonucleotide comprises 1, 2, 3, 4, or 5 or more stretches of nucleotides in a sequence that is complementary to glucagon-encoding mRNA or glucogon receptor-encoding mRNA, wherein each stretch of complementary continguous nucleotides is at least at least 4, 5, 6, 7, 8, 9, 10, 11, 12, 13, 14, 15, 16, 17, 18, 19, 20, 21, 22, 23, 24, 25 or more nucleotides in length.

In some embodiments, the oligonucleotide is modified to increase its stability in vivo.

Aspects of the present invention relate to the use of compounds effective to increase free RAPTOR can be used treat metabolic diseases such as hypertriglyceridemia, hyperinsulinemia, Type 2 Diabetes, or fatty liver disease.

Aspects of the present invention relate to the use of compounds effective to increase PHLPP2 can be used treat metabolic diseases such as hypertriglyceridemia, hyperinsulinemia, Type 2 Diabetes, or fatty liver disease.

The present invention also provides a method of reducing a subject's hepatic and plasma triglyceride levels comprising administering to a subject in need thereof a pharmaceutical composition comprising a pharmaceutical carrier and a compound that prevents PHLPP2 degradation in liver cells in an amount effective to reduce the subject's hepatic and plasma triglyceride levels.

In some embodiments, the pharmaceutical composition inhibits Glucagon signaling.

The present invention also provides a method of reducing a subject's hepatic and plasma triglyceride levels comprising administering to a subject in need thereof a pharmaceutical composition comprising a pharmaceutical carrier and a compound that inhibits Glucagon signaling in liver cells in an amount effective to reduce the subject's hepatic and plasma triglyceride levels.

In some embodiments, the pharmaceutical composition reduces PHLPP2 degradation.

In some embodiments, the pharmaceutical composition increases free Raptor in the liver cells.

The present invention also provides a method of reducing a subject's hepatic and plasma triglyceride levels comprising administering to a subject in need thereof a pharmaceutical composition comprising a pharmaceutical carrier and a compound that decreases KCTD17 expression in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that decreases PHLPP2 phosphorylation at Serine 1119 and Serine 1210 residues in liver cells in an amount effective to reduce the subject's hepatic and plasma triglyceride levels.

In some embodiments, the pharmaceutical composition decreases KCTD17 expression in liver cells.

The present invention also provides a method of reducing a subject's hepatic and plasma triglyceride levels comprising administering to a subject in need thereof a pharmaceutical composition comprising a pharmaceutical carrier and a compound that decreases KCTD17 expression in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that decreases PHLPP2 phosphorylation at Serine 1119 and Serine 1210 residues in liver cells in an amount effective to reduce the subject's hepatic and plasma triglyceride levels.

In some embodiments, the pharmaceutical composition decreases KCTD17 expression in liver cells.

In some embodiments, a compound that is capable of inhibiting a Glucagon signaling is administered to the subject.

Inhibiting Srebp1c

As used herein, “inhibiting Srebp1c” includes inhibiting the translocation of SREBP, reducing SREBP activation, inhibiting SREBP processing, or reducing the expression of SREBP. Further examples may be found in Kamisuki et al., 2009; Tang et al., 2011; Zhao et al., 2014; Hawkins et al., 2008; and Wu et al., 2014.

In some embodiments, the pharmaceutical composition comprises an Srebp1c inhibitor, thereby decreasing the expression of genes involved in lipid metabolism.

In some embodiments, the pharmaceutical composition comprises an Srebp1c inhibitor, thereby reducing the subject's hepatic and plasma triglyceride levels.

In some embodiments, the Srebp1c inhibitor is 24, 25-expoxycholesterol, and 25-HC.

In some embodiments, the Srebp1c inhibitor is Fatostatin, Betulin, BF-175, PF-429242, or BioE-1115.

In some embodiments, the compound that is capable of inhibiting Srebp1c is an antisense oligonucleotide, an RNAi molecule, or a ribozyme that targets Srebp1c mRNA and reduces Srebp1c expression.

In some embodiments, a compound that is capable of inhibiting Srebp1c is administered to the subject.

### Inhibiting KCTD17

As used herein, “inhibiting KCTD17” includes inhibiting the translocation of KCTD17, reducing KCTD17 activation, inhibiting KCTD17 processing, or reducing the expression of KCTD17.

In some embodiments, the pharmaceutical composition comprises an KCTD17 inhibitor, thereby decreasing the expression of genes involved in lipid metabolism.

In some embodiments, the pharmaceutical composition comprises an KCTD17 inhibitor, thereby reducing the subject's hepatic and plasma triglyceride levels.

In some embodiments, the compound that is capable of inhibiting KCTD17 is an antisense oligonucleotide, an RNAi molecule, or a ribozyme that targets KCTD17 mRNA and reduces KCTD17 expression.

In some embodiments, a compound that is capable of inhibiting KCTD17 is administered to the subject.

### Threshold Information for Diagnostic Utility of KCTD17

Steatosis scores >2 correspond to relatively severe steatosis; further, NAFLD activity score (NAS) >3 indicates a very high risk of NASH. Evaluation of qPCR data from patients undergoing liver biopsy for suspected NAFLD/NASH revealed a significant positive correlation between KCTD17 expression and NASH as well as the steatosis sub-score (FIGS. 80A and 80B). Further, a 1.9-fold increase in hepatic KCTD17 expression (as compared to the mean for the population) predicted a steatosis score >2 and NAS >3, and may define a diagnostic threshold in the evaluation of patients for advanced steatosis and high risk for NASH.

In some embodiments, the subject afflicted with elevated triglyceride levels has increased hepatic KCTD17 expression.

In some embodiments, the subject at risk of developing elevated triglyceride levels has increased hepatic KCTD17 expression.

In some embodiments, the increased hepatic KCTD17 expression is about 1.5, 1.6, 1.7, 1.8, 1.9, 2.0, 2.2, 2.3, 2.3 fold or more higher than the KCTD17 expression in a reference subject.

In some embodiments, the increased hepatic KCTD17 expression is about 1.9 fold or more higher than the KCTD17 expression in a reference subject.

In some embodiments, the increased hepatic KCTD17 expression is about 1.9 fold higher than the KCTD17 expression in a reference subject.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Methods

### Experimental Animals

Male wild-type C57/BL/6 mice, fed on standard chow or HFD (Harlan Laboratories TD.06414), were purchased from Jackson Labs. We injected AAV8-TBG-GFP or AAV8-TBG-Cre (Penn Vector Core) into male Raptorfl/fl (Jackson Laboratory; stock number 013188) mice, and characterized the mice 2 weeks after adeno-associated virus (AAV) injection. Number of animals used in experiments was chosen to ensure adequate power to detect experimental difference with alpha set to 0.05. All animal experiments were conducted in accordance with guidelines of the Columbia University Institutional Animal Care and Use Committee.

### Metabolic Analyses

Blood glucose was measured using a glucose meter (OneTouch), and plasma insulin by mouse insulin ELISA kit (Mercodia). Glucose tolerance tests were performed by intraperitoneal injection of 2 g per kg body weight glucose after a 16 h fast. Hepatic lipids were extracted (Folch et al., 1957), and plasma and hepatic triglyceride, Cholesterol E and NEFA were measured using a colorimetric assay from Thermo or Wako Chemicals, according to the manufacturer's protocol. We measured de novo lipogenesis as previously described (Zhang et al., 2006).

### Adenovirus Studies

The GFP, Raptor and PHLPP1/2 adenoviruses have been described (Miyamoto et al., 2010; Pajvani et al., 2013). The HA-Flag-PHLPP2 adenovirus was constructed by inserting a Flag sequence into HA-PHLPP2 vector (Addgene #22403, courtesy of Alexandra Newton) and adenoviruses encoding HA-Flag-PHLPP2 generated by Welgen, Inc. (Worcester, Mass.). Mouse shPhlpp1 or 2 target sequences were selected among three candidate sequences, respectively and adenoviruses encoding shPhlpp1 or 2 generated by Welgen, Inc. For in vivo studies, we injected 5×108 or 2.5×108 purified viral particles per g body weight; we performed metabolic analysis on days 3-5 and euthanized the mice at day 8 or 10 after injection. Infection with adenovirus in primary hepatocytes or Hepa1c1c7 cells was performed at 2.5, 5, or 10 multiplicity of infection (MOI).

### Primary Hepatocyte Cultures

We isolated and cultured primary mouse hepatocytes as previously described (Kim et al., 2012). For inhibitor experiments, we treated hepatocytes with 20 nM rapamycin (Cell Signaling), 250 nM Torinl, or vehicle for either 1 h or 24 h.

### Western Blotting and Immunoprecipitation

Tissues and cells were lysed in 0.3% CHAPS lysis buffer (Kim et al., 2002) unless otherwise stated, and whole cell lysates obtained by centrifugation. Immunoblots were conducted on samples randomly chosen within each experimental cohort with antibodies against Raptor, Akt, p-Akt (S473), p-Akt (T308), p-Akt1 (S473), p-Akt2 (S474), p-Akt (T450), p-Akt substrate, PKCα, p-GSK3β (S9), GSK3β, mTOR, Rictor, GβL, p-S6 (S240/244), S6, β-TrCP and β-actin from Cell Signaling; mTOR and α-tubulin from SantaCruz Biotechnology Inc.; PHLPP1 and PHLPP2 from Bethyl Laboratories Inc.; as well as a polyclonal antibody against Raptor from Invitrogen. For immunoprecipitation (IP) experiments, liver or cellular lysate was incubated with anti-mTOR or PHLPP2 antibody immobilized on Protein A or G-Sepharose (Invitrogen).

### Gel Filtration Chromatography

Livers from three mice per group were pooled and lysed in hypotonic buffer (40 mM Tris-HCl, pH 7.5) with protease inhibitor (Pierce) by repeated passage in a 27-gauge needle. Resultant lysates were repeatedly centrifuged at 14,000 rpm for 15 min, and the supernatant fraction filtered through a PVDF membrane prior to application to a Superose 6 column (Amersham Biosciences) calibrated with the Gel Filtration Marker Kit (Sigma), and eluted with 40 mM Tris-HCl (pH 7.5) and 150 mM NaCl.

### Crosslinking Assays

Liver was lysed in 1% Triton X-100 lysis buffer (Kim et al., 2002) with or without 2.5 mg/ml DSP or DSS, and incubated for 2 h on ice. Crosslinking was quenched with 1 M Tris-HCl (pH 7.5) for 30 min on ice. For in vitro crosslinking assays, DSP or DSS were added to a final concentration of 1 mg/ml culture medium, and quenched by Tris-HCl as above. Equal protein (BCA assay, Thermo) was then subjected to Western blot, with or without IP.

### Quantitative RT-PCR

We isolated RNA with TRIzol (Invitrogen) or an RNeasy Mini kit (Qiagen), synthesized cDNA with High-Capacity cDNA Reverse Transcription kit (Applied Biosystems) and performed quantitative RT-PCR with a GoTaq SYBR Green qPCR kit (Promega) in a CFX96 Real-Time PCR detection system (Bio-Rad).

### In Vitro Akt Kinase Assay

Liver lysates were immunoprecipitated with anti-Akt antibody (sepharose bead conjugated); bound protein was incubated with a recombinant GSK-3 fusion protein substrate in kinase buffer for 30 min at 30° C., prior to termination with SDS loading buffer and SDS-PAGE, with p-GSK3β (S9) detected by immunoblot.

### Statistical Analysis

We performed comparisons using two-way ANOVA. All data are shown as the means±s.e.m.

### Terms

As used herein, “about” in the context of a numerical value or range means±10% of the numerical value or range recited or claimed, unless the context requires a more limited range.

As used herein, “a subject afflicted with” a disease, e.g. nonalcoholic fatty liver disease, means a human patient who was been affirmatively diagnosed to have the disease.

As used herein, “effective” when referring to an amount of a compound or compounds refers to the quantity of the compound or compounds that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the physical condition of the patient, the type of subject being treated, the duration of the treatment, the nature of concurrent therapy (if any), and the specific formulations employed and the structure of the compounds or its derivatives.

In some embodiments, “a subject in need” includes a subject with elevated triglyceride levels, e.g., a subject with hepatic or plasma or serum triglyceride levels greater than 150 mg/dL, greater than 200 mg/dL, or to greater than 500 mg/dL.

In some embodiments, “a subject in need” includes a subject with decreased free Raptor, e.g., a subject afflicted with obesity.

In some embodiments, “a subject in need” encompasses, e.g., a subject with plasma or serum triglyceride level greater than 150 mg/dL, greater than 200 mg/dL, or to greater than 500 mg/dL.

In some embodiments, a subject afflicted with a metabolic disease, such as obesity, tends to have elevated triglyceride levels.

In some embodiments, a pharmaceutical composition comprises a pharmaceutical carrier and a compound.

In some embodiments, the pharmaceutical composition is manufactured, wherein the pharmaceutical composition comprises an isolated or purified naturally occurring compound.

In some embodiments, the pharmaceutical composition is manufactured, wherein the pharmaceutical composition comprises a compound which was not produced by a process in nature.

In some embodiments, a reference subject includes a subject with hepatic or plasma or serum triglyceride levels less than 150 mg/dL.

As described herein, young mice (9-week old, equivalent to an immediately post-pubertal human) tend to have “normal” Raptor levels, which decline by 24-weeks (equivalent to adulthood).

In some embodiments, a reference subject includes a subject that has not reached adulthood.

In some embodiments, a reference subject includes a subject younger than 18 years old.

In some embodiments, a reference subject is not afflicted with a metabolic disease and is younger than 18 years old.

### Methods of Increasing Raptor or PHLPP2

In some embodiments, each compound administered to the subject is, independently, an organic compound having a molecular weight less than 1000 Daltons, a polypeptide, an oligonucleotide, or a small molecule.

In some embodiments, the pharmaceutical composition which is capable of increasing PHLPP2 or free Raptor enhances expression of a gene or enhances transcription.

In some embodiments, the pharmaceutical composition which is capable of increasing free Raptor inhibits interaction of Raptor and mTORC1.

In some embodiments, a pharmaceutical composition that is capable of increasing free Raptor is administered to the subject.

In some embodiments, a pharmaceutical composition that is capable of increasing PHLPP2 is administered to the subject.

In some embodiments, a pharmaceutical composition that is capable of increasing Raptor expression is administered to the subject.

In some embodiments, a pharmaceutical composition that is capable of increasing PHLPP2 expression is administered to the subject.

### Small Molecule

A small molecule may be administered herein to increase free Raptor or increase activity of Raptor.

A small molecule may be administered herein to increase PHLPP2 or increase activity of PHLPP2.

Non-limiting examples of PHLPP2 activators such as anti-miRNA oligonucleotides to miR-190, or small-molecular or other inhibitors of the B-TrCP degradative pathway are described, for example, in the following publications: Beezhold, et al, Toxicological Sciences, 2011, and Li, Liu and Gao, Mol Cell Biology, 2009, which are hereby incorporated by reference in their entireties.

### Oligonucleotide

Non-limiting examples of oligonucleotides capable of increasing Raptor or PHLPP2 expression include antisense oligonucleotides, polynucleotides, and adenoviral vectors.

The amino acid sequence of Raptor, or KIAA1303, is accessible in public databases by the GenBank accession number Q8N122.1, and is set forth herein as SEQ ID NO: 1.

The amino acid sequence of, PH domain leucine-rich repear-containing protein phosphatase 2 (PHLPP2), is accessible in public databases by the GenBank accession number Q6ZVD8.3, and is set forth herein as SEQ ID NO: 2.

In some embodiments, the pharmaceutical composition which is capable of increasing free Raptor or PHLPP2 comprises a polynucleotide or an adenovirus.

In some embodiments, the pharmaceutical composition which is capable of increasing Raptor or PHLPP2 expression comprises a polynucleotide or an adenovirus.

Non-limiting examples of oligonucleotides capable of decreasing Glucagon signaling include antisense oligonucleotides, polynucleotides, and adenoviral vectors.

The sequence of, Glucagon (GCG), is accessible in public databases by the GenBank accession number NM_002054.4, and is set forth herein as SEQ ID NO: 3.

The amino acid sequence of Glucagon Receptor (gcgr) is accessible in public databases by the GenBank accession number L20316.1, and is set forth herein as SEQ ID NO: 4.

As used herein, “a subject afflicted with” a disease, e.g. nonalcoholic fatty liver disease, means a human patient who was been affirmatively diagnosed to have the disease.

As used herein, “a subject afflicted with” a disease, e.g. nonalcoholic fatty liver disease, means a human patient who was been affirmatively diagnosed to have the disease.

### Antisense Oligonucleotide

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of target gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modifications of gene expression can be obtained by designing antisense oligonucleotides which will form duplexes to the control, 5′, or regulatory regions of the gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (Nicholls et al., 1993, J Immunol Meth 165:81-91). An antisense oligonucleotide also can be designed to block translation of mRNA by preventing the transcript from binding to ribosomes.

Precise complementarity is not required for successful complex formation between an antisense oligonucleotide and the complementary sequence of a target polynucleotide. Antisense oligonucleotides which comprise, for example, 1, 2, 3, 4, or 5 or more stretches of contiguous nucleotides which are precisely complementary to a target polynucleotide, each separated by a stretch of contiguous nucleotides which are not complementary to adjacent nucleotides, can provide sufficient targeting specificity for a target mRNA. Preferably, each stretch of complementary contiguous nucleotides is at least 4, 5, 6, 7, 8, 9, 10, 11, 12, 13, 14, 15, 16, 17, 18, 19, 20, 21, 22, 23, 24, 25, or more nucleotides in length. Noncomplementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular target polynucleotide sequence. Antisense oligonucleotides can be modified without affecting their ability to hybridize to a target polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art.

### Ribozymes

Ribozymes are RNA molecules with catalytic activity (Uhlmann et al., 1987, Tetrahedron. Lett. 215, 3539-3542). Ribozymes can be used to inhibit gene function by cleaving an RNA sequence, as is known in the art. The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. The coding sequence of a polynucleotide can be used to generate ribozymes which will specifically bind to mRNA transcribed from the polynucleotide. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the target RNA.

Specific ribozyme cleavage sites within an RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease target gene expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or VAS element, and a transcriptional 45 on alcohol signal, for controlling transcription of ribozymes in the cells (U.S. Pat. No. 5,641,673). Ribozymes also can be engineered to provide an additional level of regulation, so that destruction of mRNA occurs only when both a ribozyme and a target gene are induced in the cells.

### RNA Interference

An interfering RNA (RNAi) molecule involves mRNA degradation. The use of RNAi has been described in Fire et al., 1998, Carthew et al., 2001, and Elbashir et al., 2001, the contents of which are incorporated herein by reference.

Interfering RNA or small inhibitory RNA (RNAi) molecules include short interfering RNAs (siRNAs), repeat-associated siRNAs (rasiRNAs), and micro-RNAs (miRNAs) in all stages of processing, including shRNAs, pri-miRNAs, and pre-miRNAs. These molecules have different origins: siRNAs are processed from double-stranded precursors (dsRNAs) with two distinct strands of base-paired RNA; siRNAs that are derived from repetitive sequences in the genome are called rasiRNAs; miRNAs are derived from a single transcript that forms base-paired hairpins. Base pairing of siRNAs and miRNAs can be perfect (i.e., fully complementary) or imperfect, including bulges in the duplex region.

Interfering RNA molecules encoded by recombinase-dependent transgenes of the invention can be based on existing shRNA, siRNA, piwi-interacting RNA (piRNA), micro RNA (miRNA), double-stranded RNA (dsRNA), antisense RNA, or any other RNA species that can be cleaved inside a cell to form interfering RNAs, with compatible modifications described herein.

As used herein, an “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a shRNA may form a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

“RNAi-expressing construct” or “RNAi construct” is a generic term that includes nucleic acid preparations designed to achieve an RNA interference effect. An RNAi-expressing construct comprises an RNAi molecule that can be cleaved in vivo to form an siRNA or a mature shRNA. For example, an RNAi construct is an expression vector capable of giving rise to a siRNA or a mature shRNA in vivo. Non-limiting examples of vectors that may be used in accordance with the present invention are described herein and will be well known to a person having ordinary skill in the art. Exemplary methods of making and delivering long or short RNAi constructs can be found, for example, in WO01/68836 and WO01/75164.

### Use of RNAi

RNAi is a powerful tool for in vitro and in vivo studies of gene function in mammalian cells and for therapy in both human and veterinary contexts. Inhibition of a target gene is sequence-specific in that gene sequences corresponding to a portion of the RNAi sequence, and the target gene itself, are specifically targeted for genetic inhibition. Multiple mechanisms of utilizing RNAi in mammalian cells have been described. The first is cytoplasmic delivery of siRNA molecules, which are either chemically synthesized or generated by DICER-digestion of dsRNA. These siRNAs are introduced into cells using standard transfection methods. The siRNAs enter the RISC to silence target mRNA expression.

Another mechanism is nuclear delivery, via viral vectors, of gene expression cassettes expressing a short hairpin RNA (shRNA). The shRNA is modeled on micro interfering RNA (miRNA), an endogenous trigger of the RNAi pathway (Lu et al., 2005, Advances in Genetics 54: 117-142, Fewell et al., 2006, Drug Discovery Today 11: 975-982). Conventional shRNAs, which mimic pre-miRNA, are transcribed by RNA Polymerase II or III as single-stranded molecules that form stem-loop structures. Once produced, they exit the nucleus, are cleaved by DICER, and enter the RISC as siRNAs.

Another mechanism is identical to the second mechanism, except that the shRNA is modeled on primary miRNA (shRNAmir), rather than pre-miRNA transcripts (Fewell et al., 2006). An example is the miR-30 miRNA construct. The use of this transcript produces a more physiological shRNA that reduces toxic effects.

The shRNAmir is first cleaved to produce shRNA, and then cleaved again by DICER to produce siRNA. The siRNA is then incorporated into the RISC for target mRNA degradation. However, aspects of the present invention relate to RNAi molecules that do not require DICER cleavage. See, e.g., U.S. Pat. No. 8,273,871, the entire contents of which are incorporated herein by reference.

For mRNA degradation, translational repression, or deadenylation, mature miRNAs or siRNAs are loaded into the RNA Induced Silencing Complex (RISC) by the RISC-loading complex (RLC). Subsequently, the guide strand leads the RISC to cognate target mRNAs in a sequence-specific manner and the Slicer component of RISC hydrolyses the phosphodiester bound coupling the target mRNA nucleotides paired to nucleotide 10 and 11 of the RNA guide strand. Slicer forms together with distinct classes of small RNAs the RNAi effector complex, which is the core of RISC. Therefore, the “guide strand” is that portion of the double-stranded RNA that associates with RISC, as opposed to the “passenger strand,” which is not associated with RISC.

It is not necessary that there be perfect correspondence of the sequences, but the correspondence must be sufficient to enable the RNA to direct RNAi inhibition by cleavage or blocking expression of the target mRNA. In preferred RNA molecules, the number of nucleotides which is complementary to a target sequence is 16 to 29, 18 to 23, or 21-23, or 15, 16, 17, 18, 19, 20, 21, 22, 23, 24, or 25.

Isolated RNA molecules can mediate RNAi. That is, the isolated RNA molecules of the present invention mediate degradation or block expression of mRNA that is the transcriptional product of the gene. For convenience, such mRNA may also be referred to herein as mRNA to be degraded. The terms RNA, RNA molecule(s), RNA segment(s) and RNA fragment(s) may be used interchangeably to refer to RNA that mediates RNA interference. These terms include double-stranded RNA, small interfering RNA (siRNA), hairpin RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the RNA or internally (at one or more nucleotides of the RNA). Nucleotides in the RNA molecules of the present invention can also comprise nonstandard nucleotides, including non-naturally occurring nucleotides or deoxyribonucleotides. Collectively, all such altered RNAi molecules are referred to as analogs or analogs of naturally-occurring RNA. RNA of the present invention need only be sufficiently similar to natural RNA that it has the ability to mediate RNAi.

As used herein the phrase “mediate RNAi” refers to and indicates the ability to distinguish which mRNA molecules are to be afflicted with the RNAi machinery or process. RNA that mediates RNAi interacts with the RNAi machinery such that it directs the machinery to degrade particular mRNAs or to otherwise reduce the expression of the target protein. In one embodiment, the present invention relates to RNA molecules that direct cleavage of specific mRNA to which their sequence corresponds. It is not necessary that there be perfect correspondence of the sequences, but the correspondence must be sufficient to enable the RNA to direct RNAi inhibition by cleavage or blocking expression of the target mRNA.

In some embodiments, an RNAi molecule of the invention is introduced into a mammalian cell in an amount sufficient to attenuate target gene expression in a sequence specific manner. The RNAi molecules of the invention can be introduced into the cell directly, or can be complexed with cationic lipids, packaged within liposomes, or otherwise delivered to the cell. In certain embodiments the RNAi molecule can be a synthetic RNAi molecule, including RNAi molecules incorporating modified nucleotides, such as those with chemical modifications to the 2′-OH group in the ribose sugar backbone, such as 2′-O-methyl (2′Ome), 2′-fluoro (2′F) substitutions, and those containing 2′Ome, or 2′F, or 2′-deoxy, or “locked nucleic acid” (LNA) modifications. In some embodiments, an RNAi molecule of the invention contains modified nucleotides that increase the stability or half-life of the RNAi molecule in vivo and/or in vitro. Alternatively, the RNAi molecule can comprise one or more aptamers, which interact(s) with a target of interest to form an aptamer:target complex. The aptamer can be at the 5′ or the 3′ end of the RNAi molecule. Aptamers can be developed through the SELEX screening process and chemically synthesized. An aptamer is generally chosen to preferentially bind to a target. Suitable targets include small organic molecules, polynucleotides, polypeptides, and proteins. Proteins can be cell surface proteins, extracellular proteins, membrane proteins, or serum proteins, such as albumin. Such target molecules may be internalized by a cell, thus effecting cellular uptake of the shRNA. Other potential targets include organelles, viruses, and cells.

As noted above, the RNA molecules of the present invention in general comprise an RNA portion and some additional portion, for example a deoxyribonucleotide portion. The total number of nucleotides in the RNA molecule is suitably less than in order to be effective mediators of RNAi. In preferred RNA molecules, the number of nucleotides is 16 to 29, more preferably 18 to 23, and most preferably 21-23.

### Adenoviral Vector

An adenoviral vecor encodes an oligonucleotide. The use of adenoviral vectors in gene therapy and tissue-specific targeting has been described in Beatty and Curiel, 2012, Barnett et al., 2002, and Rots et al., 2003, the contents of which are incorporated herein by reference.

### Methods of Administration

“Administering” compounds in embodiments of the invention can be effected or performed using any of the various methods and delivery systems known to those skilled in the art. The administering can be, for example, intravenous, oral, intramuscular, intravascular, intra-arterial, intracoronary, intramyocardial, intraperitoneal, and subcutaneous. Other non-limiting examples include topical administration, or coating of a device to be placed within the subject.

### Injectable Drug Delivery

Injectable drug delivery systems may be employed in the methods described herein include solutions, suspensions, gels.

### Oral Drug Delivery

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

For oral administration in liquid dosage form, a PHLPP2 or Raptor activator may be combined with any oral, non-toxic, pharmaceutically acceptable inert carrier such as ethanol, glycerol, water, and the like.

### Pharmaceutically Acceptable Carrier

The compounds used in embodiments of the present invention can be administered in a pharmaceutically acceptable carrier. As used herein, a “pharmaceutically acceptable carrier” is a pharmaceutically acceptable solvent, suspending agent or vehicle, for delivering the compounds to the subject. The carrier may be liquid or solid and is selected with the planned manner of administration in mind. Liposomes such as small unilamellar vesicles, large unilamallar vesicles, and multilamellar vesicles are also a pharmaceutically acceptable carrier. Liposomes can be formed from a variety of phospholipids, such as cholesterol, stearylamine, or phosphatidylcholines. The compounds may be administered as components of tissue-targeted emulsions. Examples of lipid carriers for antisense delivery are disclosed in U.S. Pat. Nos. 5,855,911 and 5,417,978, which are incorporated herein by reference. The compounds used in the methods of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

A compound of the invention can be administered in a mixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297, issued Sep. 2, 1975.

### Tablets

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

### Specific Administration to Liver

Embodiments of the invention relate to specific administration to the liver or hepatocytes.

In some embodiments, a compound may specifically target the liver.

In some embodiments, a compound may specifically target hepatocytes.

In some embodiments, a compound may be specifically targeted to the liver by coupling the compound to ligand molecules, targeting the compound to a receptor on a hepatic cell, or administering the compound by a bio-nanocapsule.

A compound of the invention can also be administered by coupling of ligand molecules, such as coupling or targeting moieties on preformed nanocarriers, such as (PGA-PLA nanoparticles, PLGA nanoparticles, cyclic RGD-doxorubicin-nanoparticles, and poly(ethylene glycol)-coated biodegradable nanoparticles), by the post-insertion method, by the Avidin-Biotin complex, or before nanocarriers formulation, or by targeting receptors present on various hepatic cell, such as Asialoglycoproein receptor (ASGP-R), HDL-R, LDL-R, IgA-R, Scavenger R, Transferrin R, and Insulin R, as described in: Mishra et al., (2013) Efficient Hepatic Delivery of Drugs: Novel Strategies and Their Significance, BioMed Research International 2013: 382184, dx.doi.org/10.1155/2013/382184, the entire contents of which are incorporated herein by reference.

A compound of the invention can also be administered by bio-nanocapsule, as described in: Yu et al., (2005) The Specific delivery of proteins to human liver cells by engineered bio-nanocapsules, FEBS Journal 272: 3651-3660, dx.doi.org/10.1111/j.1742-4658.2005.04790.x, the entire contents of which are incorporated herein by reference.

In some embodiments, an oligonucleotide specifically targets the liver.

In some embodiments, an oligonucleotide specifically targets hepatocytes.

Antisense oligonucleotides of the invention can also be targeted to hepatocytes, as described in: Prakash et al., (2014) Targeted delivery of antisense oligonucleotides to hepatocytes using triantennary N-acetyl galactosamine improves potency 10-fold in mice, Nucleic Acids Research 42(13): 8796-8807, dx.doi.org/10.1093/nar/gku531, the entire contents of which are incorporated herein by reference.

As used herein, the term “effective amount” refers to the quantity of a component that is sufficient to treat a subject without undue adverse side effects (such as toxicity, irritation, or allergic response) commensurate with a reasonable benefit/risk ratio when used in the manner of this invention, i.e. a therapeutically effective amount. The specific effective amount will vary with such factors as the particular condition being treated, the physical condition of the patient, the type of subject being treated, the duration of the treatment, the nature of concurrent therapy (if any), and the specific formulations employed and the structure of the compounds or its derivatives.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2^{nd} Edition (1976); Remington's Pharmaceutical Sciences, 17^{th} ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The dosage of a compound of the invention administered in treatment will vary depending upon factors such as the 56onalcoholic56ics characteristics of the compound and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds of the invention may comprise a compound alone, or mixtures of a compound with additional compounds used to treat cancer. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the eye, all using dosage forms well known to those of ordinary skill in the pharmaceutical arts.

In an embodiment, the pharmaceutical composition may be administered once a day, twice a day, every other day, once weekly, or twice weekly.

A subject's triglyceride level may be expressed herein as hepatic triglyceride or plasma triglyceride or serum triglyceride.

Where a range is given in the specification it is understood that the range includes all integers and 0.1 units within that range, and any sub-range thereof. For example, a range of 1 to 5 is a disclosure of 1.0, 1.1, 1.2, etc.

This invention will be better understood by reference to the Examples which follow, but those skilled in the art will readily appreciate that the specific experiments detailed are only illustrative of the invention as described more fully in the claims which follow thereafter.

### Combination Therapy

The administration of two drugs to treat a given condition, such as non-alcoholic fatty liver disease (NAFLD), raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug. (Guidance for Industry, 1999) Thus, when two drugs are administered to treat the same condition, it is unpredictable whether each will complement, have no effect on, or interfere with the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side effect profile of each drug.

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs. (Guidance for Industry, 1999)

### Example 1

### Identification of Post-Translational Modifications (PTMs) on PHLPP2 Protein

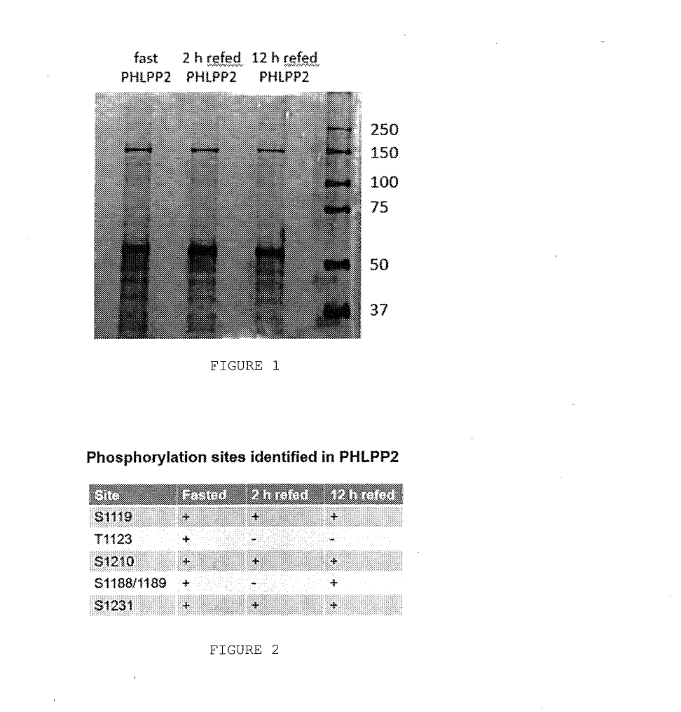

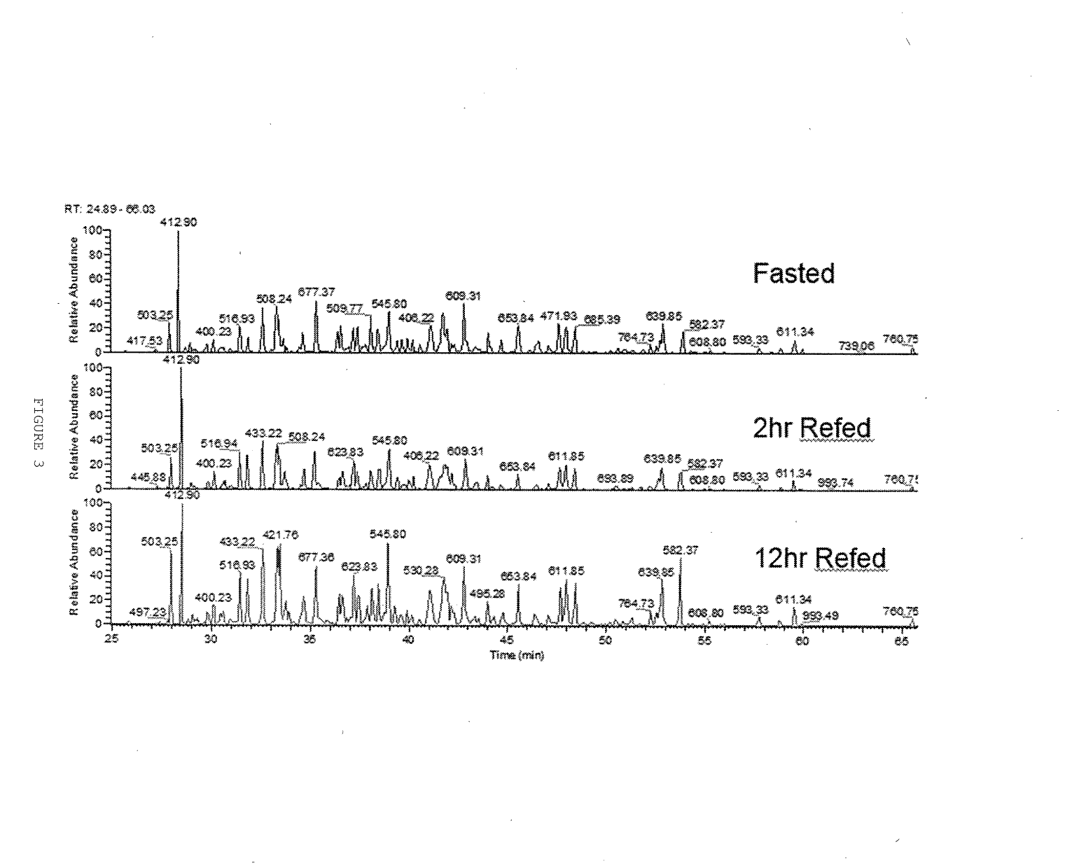

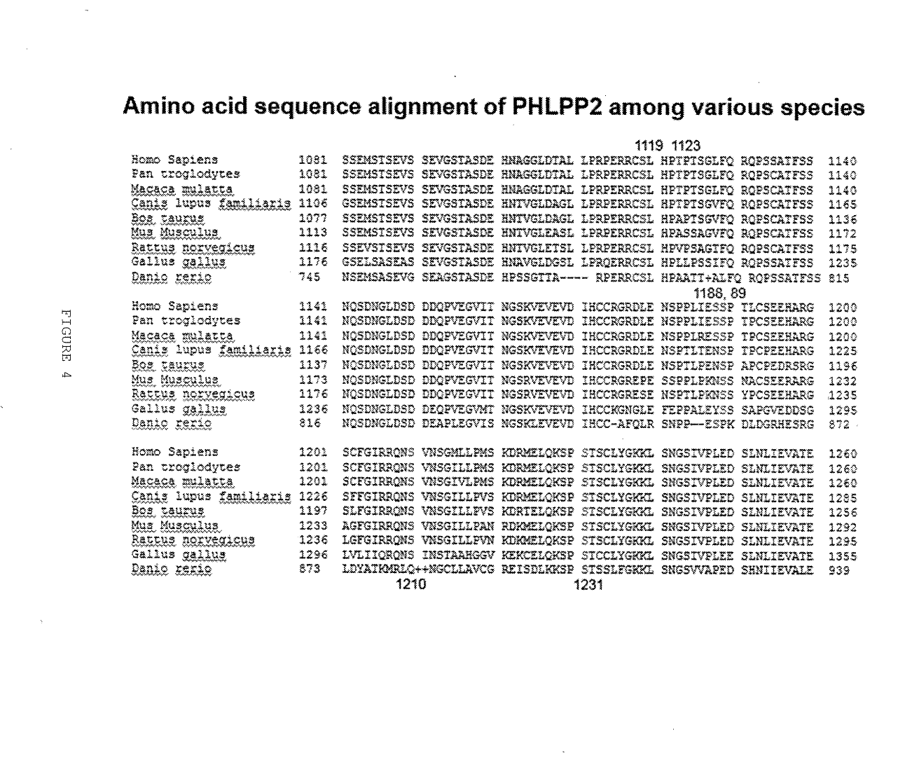

Reduced hepatic PHLPP2 protein levels, but not Phlpp2 gene expression, were observed in aging/obesity and short-term refeeding, suggesting altered PHLPP2 stability in the insulin-resistant or insulin-rich liver, consistent with data showing regulated PHLPP2 ubiquitination and proteasomal degradation. It was next hypothesized that PHLPP2 protein stability was affected by nutrient- or hormone-mediated post-translational modifications (PTMs), and an in vivo unbiased liquid chromatography-tandem mass spectrometry (LC/MS-MS) screen by transducing C57BL/6 wild-type (WT) mice with Ad-HA-Flag-PHLPP2 followed by sequential anti-HA→anti-FLAG IP was performed (FIG. 1). With this approach, five novel phosphorylation sites were identified at the functionally undefined C-terminal region between the PP2C domain and PDZ-binding motif of PHLPP2 (FIG. 2-4).

### PHLPP2 Ser1119 and Ser 1210 are Phosphorylated in Response to Glucagon

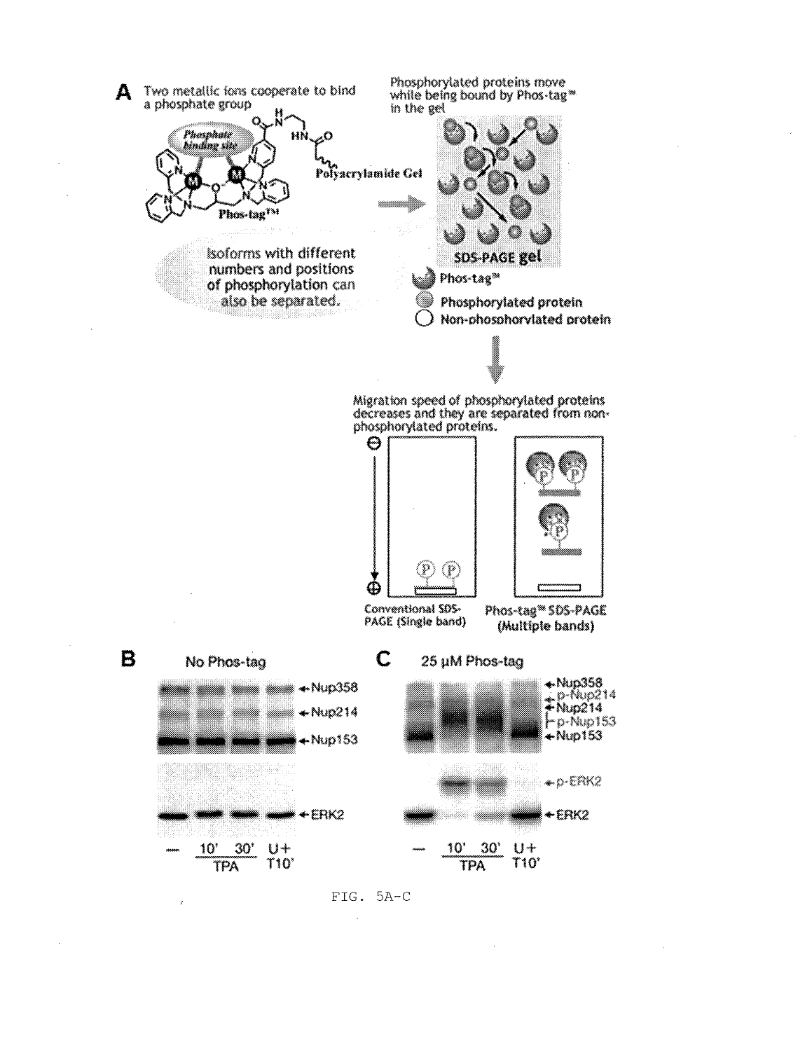

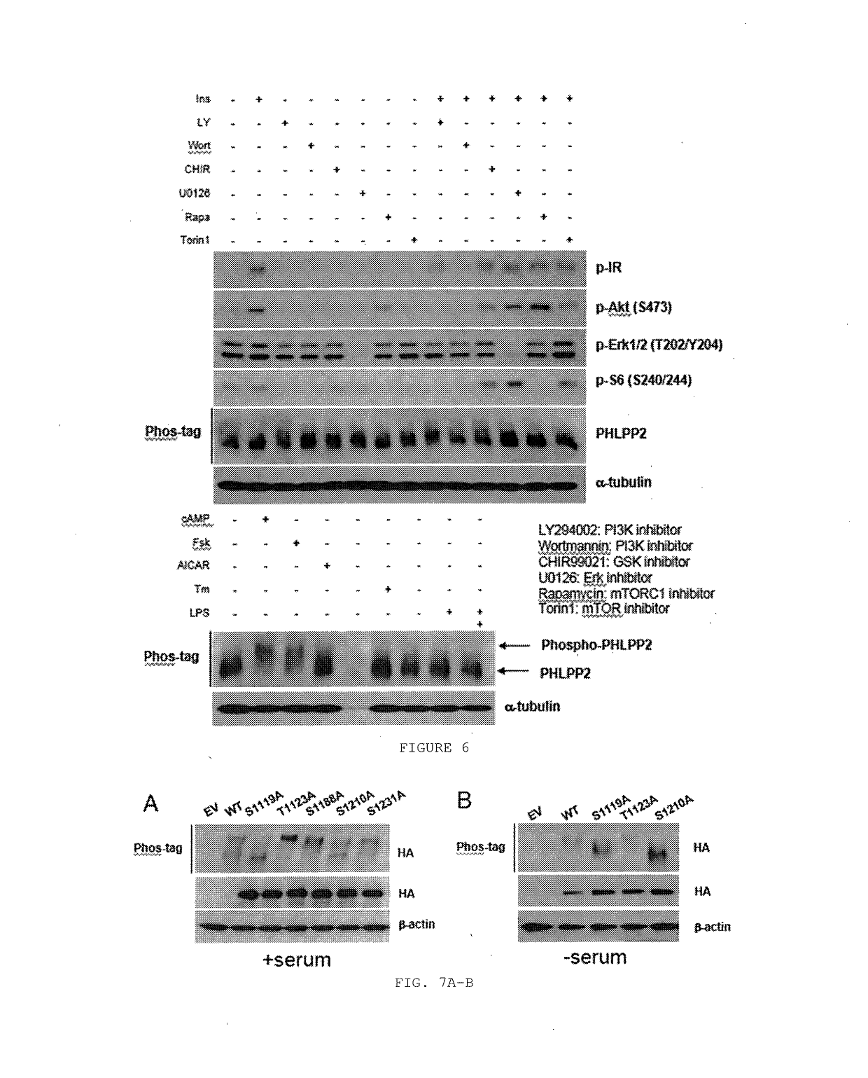

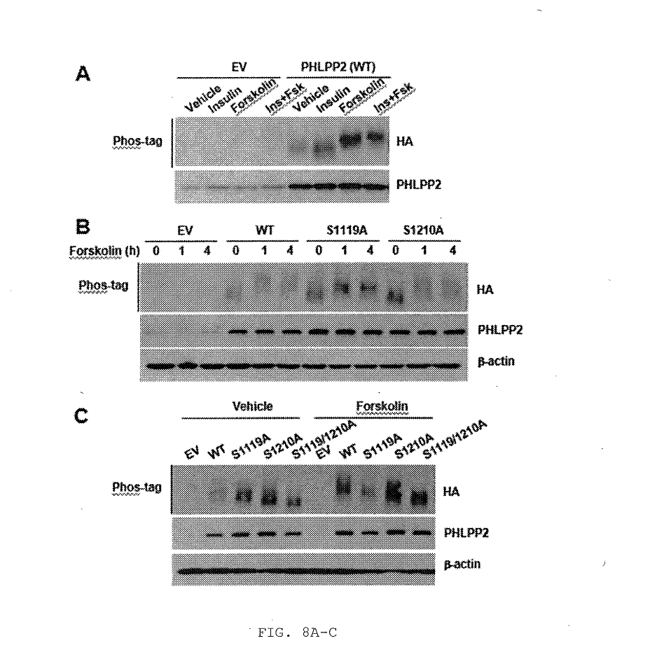

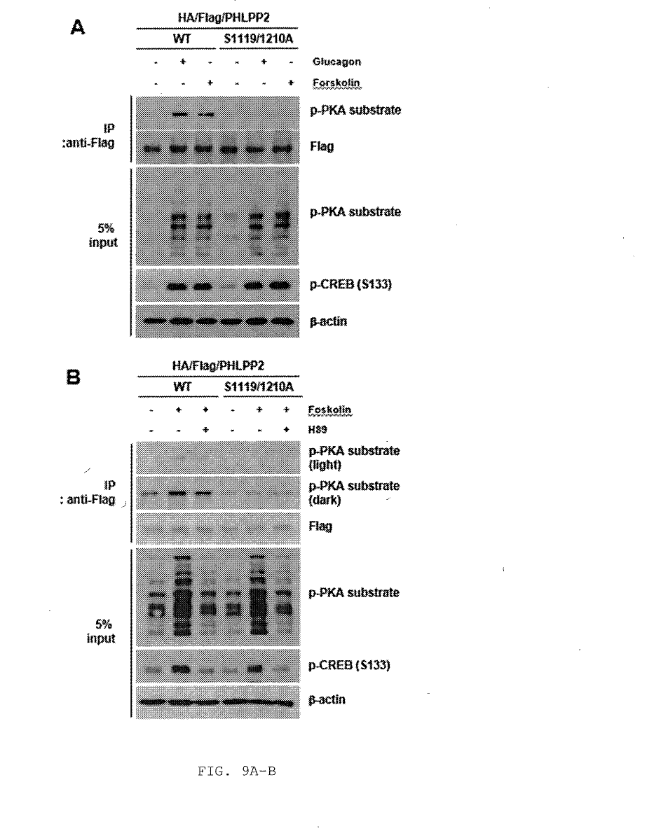

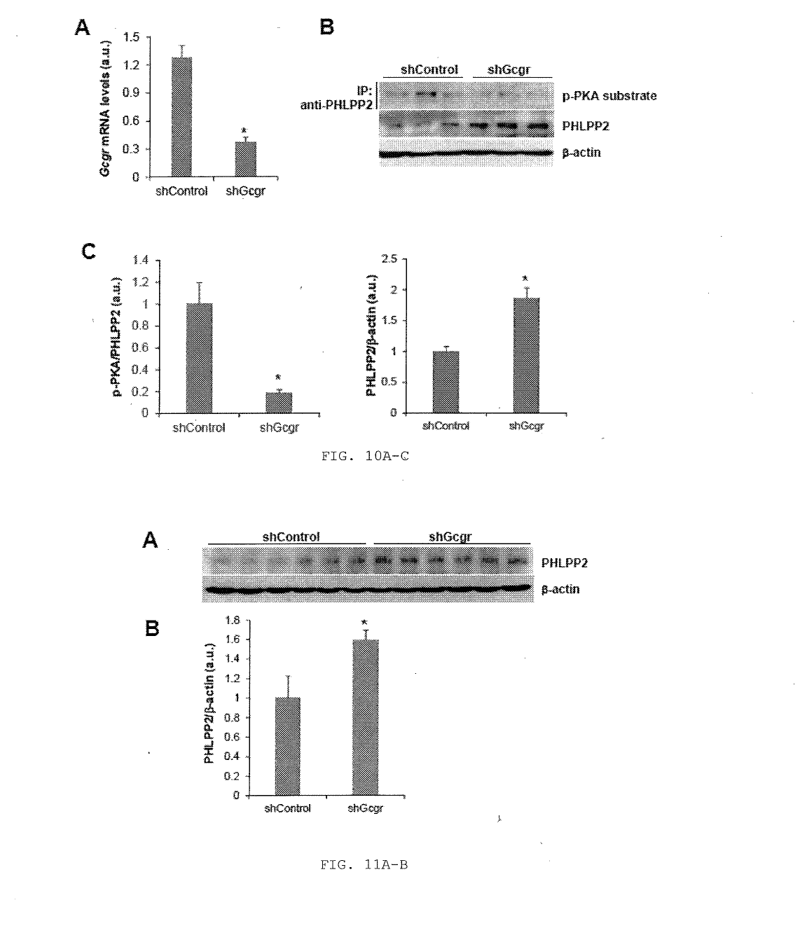

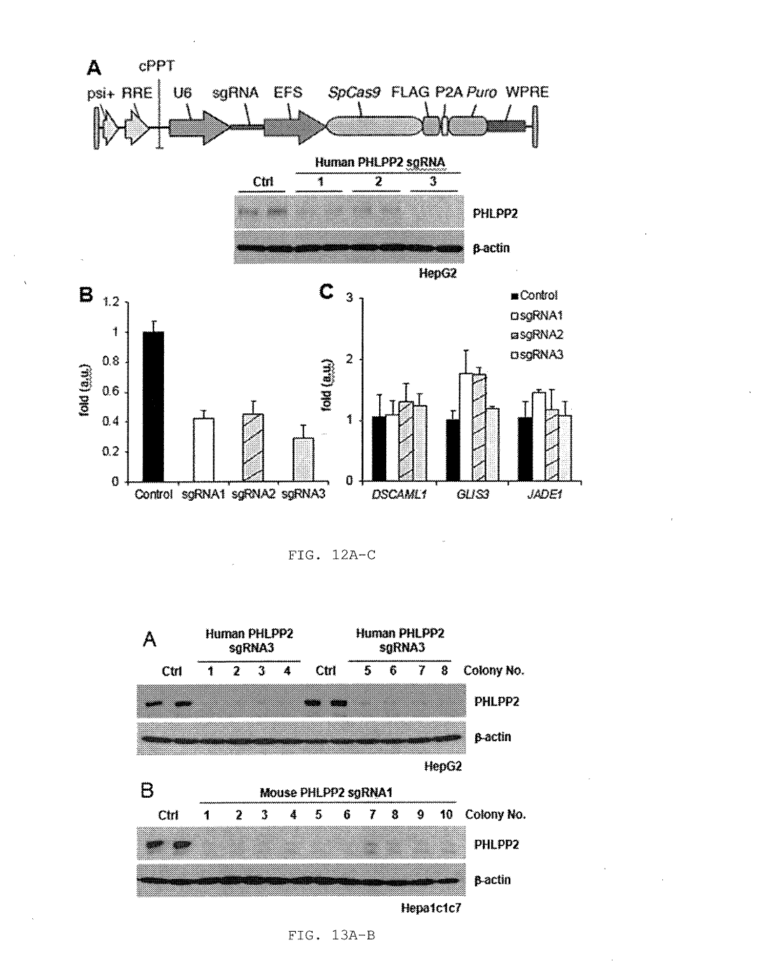

To identify the signal transduction pathways involved in PHLPP2 phosphorylation, a Phos-tag-based mobility shift assay was used, which magnifies the difference in migration speed of phosphorylated and non-phosphorylated proteins (FIG. 5A-C), and it was found that activation of protein kinase A (PKA) signaling by treatment with either cAMP, or forskolin which activates adenylate cyclase to stimulates cAMP production, increased PHLPP2 phosphorylation (FIG. 6). Next, to validate LC/MS-MS-based phospho-peptide mapping results, each identified Ser/Thr site was individually mutated to Alanine, then transfected WT or Ala-mutant PHLPP2 in hepatocytes, and Ser1119 and Ser1210 phosphorylations was confirmed (FIG. 7A-B). Next, it was found that forskolin could similarly induce phosphorylation overexpressed PHLPP2 in primary hepatocytes (FIG. 8A), essential to test the effects of Ala-mutant PHLPP2 in vitro. Interestingly, WT, S1119A, and S1210A mutant of PHLPP2 were all phosphorylated with forskolin treatment, suggesting that both S1119 and S1210 sites might be phosphorylated by PKA (FIG. 8B). Thus, double mutants were generated, such as S1119/1210 and it was found that ablation of both sites rendered the S1119A/S1210A mutant insensitive to forskolin-induced phosphorylation (FIG. 8C). These data matched in silico analysis of consensus substrate sites for mammalian kinases, as both Ser1119 and Ser1210 are exact matches for the cyclic AMP-PKA consensus site. To investigate the effect of PKA on PHLPP2 phosphorylation in primary hepatocytes, either WT or S1119/1210A mutant of PHLPP2 was immunoprecipitated from cell extracts treated with glucagon or forskolin, and then subjected to immunoblot analysis with an antibody specific for phosphorylated PKA substrate proteins. PKA-mediated phosphorylation of WT, but not S1119A/S1210A mutant PHLPP2 was robustly increased following glucagon or forskolin treatment of primary hepatocytes. In addition, forskolin-mediate PHLPP2 phosphorylation was reduced by pretreatment of PKA inhibitor, H89, proving that PKA stimulates PHLPP2 phopshorylation (FIG. 9A-B).

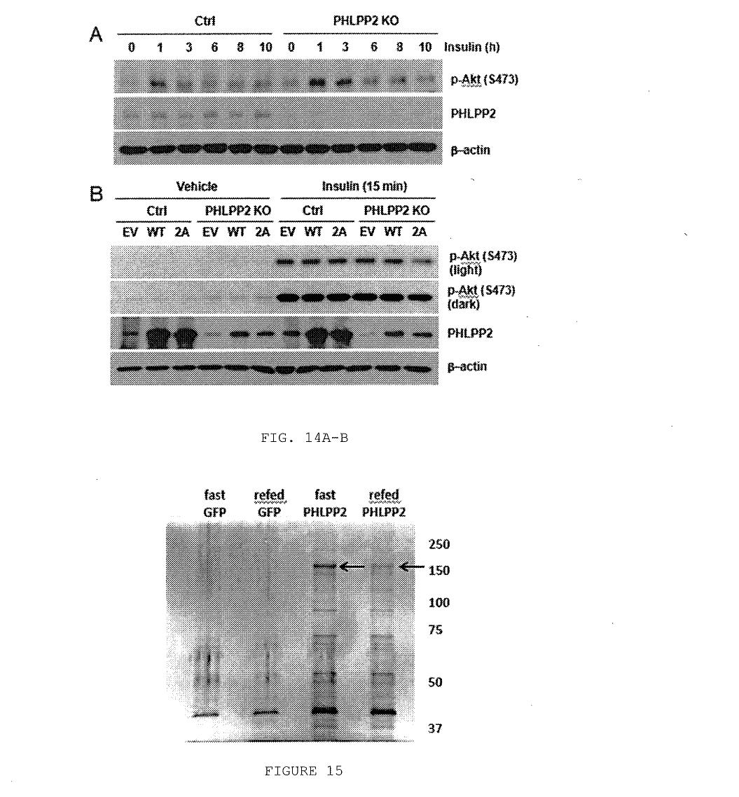

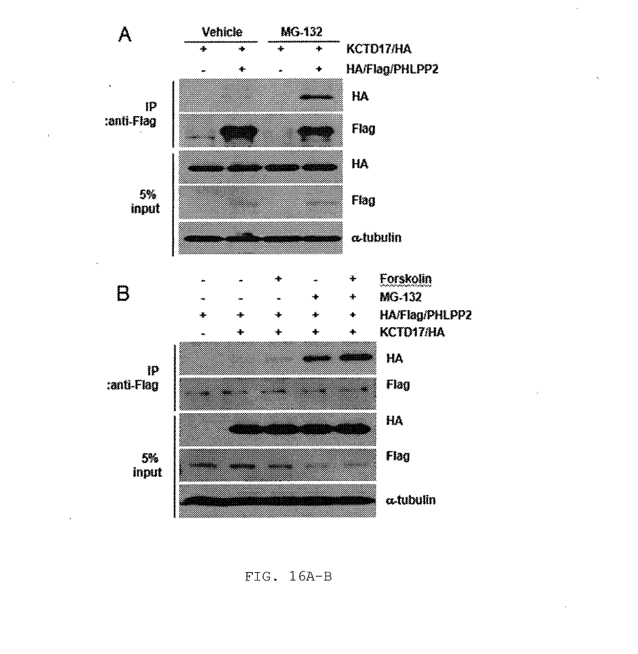

These data suggest that glucagon may induce PHLPP2 phosphorylation and thus degradation, and that inhibitors of glucagon signaling would increase PHLPP2 levels. To test this concept in vivo, adenoviruses encoding shRNA were transduced to the glucagon receptor (or a control shRNA) to either genetically-induced (db/db) mice or diet-induced obese (DIO) mice. As hypothesized, knockdown of liver glucagon signaling rescues the lower PHLPP2 levels seen in obesity (FIGS. 10A-C and FIGS. 11A-B). This suggests that inhibitors of glucagon signaling will similarly increase the pathologically lower PHLPP2 levels in the obese state, and reduce hepatic and plasma triglyceride.

### PHLPP2 Knockout (KO) Cells Show Prolonged Insulin Signaling.

To determine whether PHLPP2 is necessary to terminate insulin signaling, PHLPP2 KO HepG2 hepatoma cells were generated using the CRISPR (Clustered Regularly Interspaced Short Palindromic Repeats)-Cas9 system. Using three different single guide RNA (sgRNA) for PHLPP2, significantly reduced PHLPP2 protein and mRNA levels were observed, without effects on off-target gene expression (FIG. 12A-C). Next, HepG2 stable cell lines were generated expressing the most efficient PHLPP2 sgRNA (#3) sgRNA3 (FIG. 13A) and, in parallel, a mouse hepatoma cell lines, Hepa1c1c7 cells (FIG. 13B). It was found that PHLPP2-deficient cells showed sustained insulin signaling as compared to control cells, as judged by Akt phosphorylation (FIG. 14A). Next, PHLPP2 levels were “rescued” in these CRISPR-induced knockout cell lines with either WT or S1119/1210 A PHLPP2, and found that only mutant PHLPP2 prevented acute insulin-induced Akt phosphorylation (FIG. 14B), indicating that PKA-mediated phosphorylation is likely-responsible for decreased insulin-mediated Akt phosphorylation.

### Identification of Novel PHLPP2 Interaction Proteins

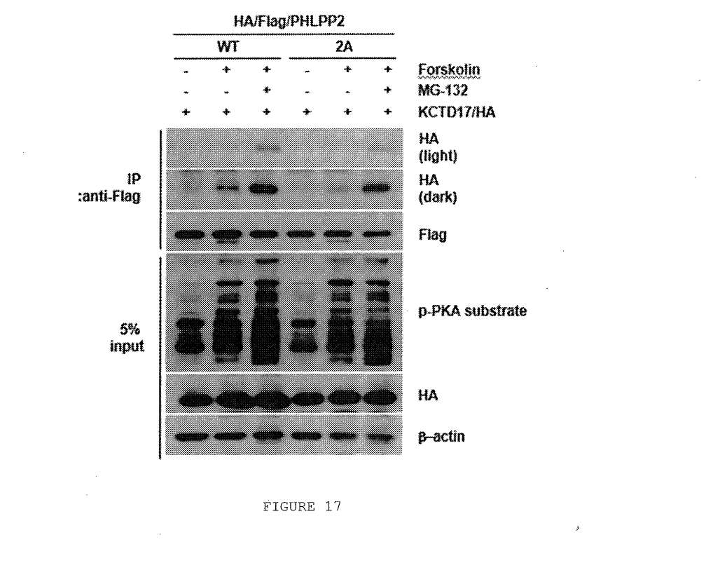

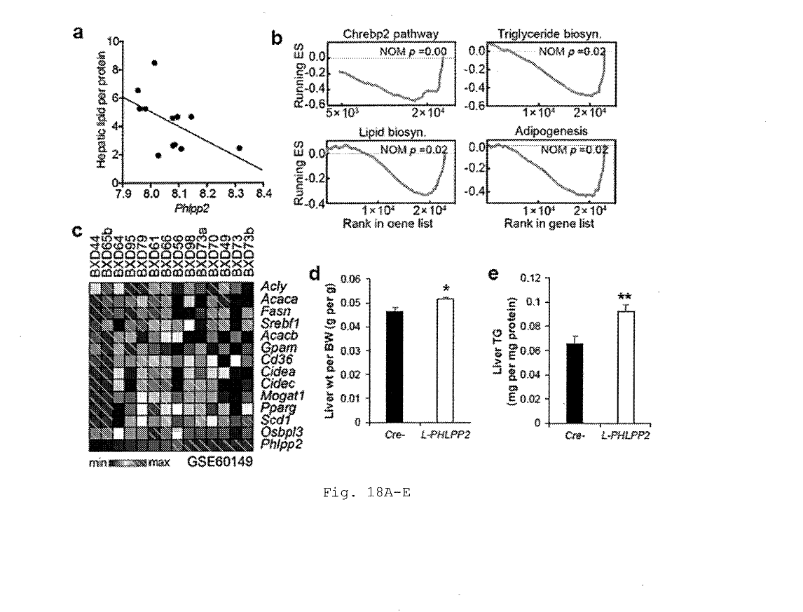

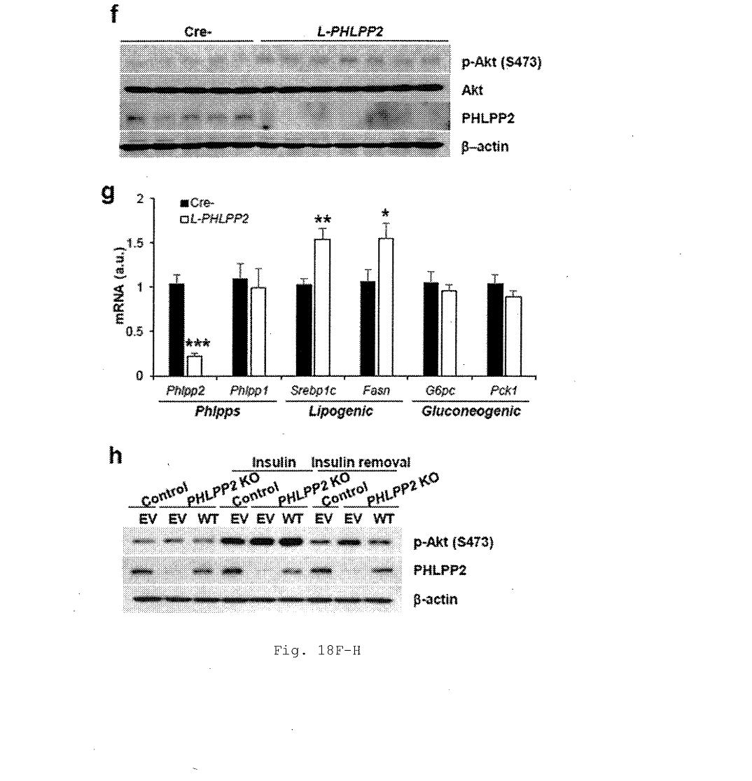

To further identify the regulatory mechanism by which PHLPP2 levels decrease in aging and obese liver through proteasomal degradation, LC/MS-MS analysis was repeated to determine novel PHLPP2 binding partners (FIG. 15), and identified KCTD17 (potassium channel tetramerization domain containing 17) as a strong PHLPP2 interactor. Next, PHLPP2 was confirmed to associate with KCTD17, but in a proteasome-dependent manner (FIG. 16A). Consistent with the notion that S1119/S120 phosphorylation regulates PHLPP2 stability by proteasomal degradation, the association of endogenous PHLPP2 with KCTD17 was significantly enhanced by treatment of forskolin, and even more strongly with simultaneous treatment with forskolin and the proteasome inhibitor, MG132 (FIG. 16B). Finally, and as hypothesized, while WT PHLPP2 associates with KCTD17 with forskolin and MG132 treatment, S1119/1210A mutant PHLPP2 showed markedly lower KCTD17 binding (FIG. 17). In sum, these data indicate that KCTD17 is a novel PHLPP2 interacting protein, which may regulate PHLPP2 degradation in a phosphorylation/proteasome-dependent manner.

### Example 2

### KCTD17 Targets PHLPP2 for Degradation to Induce NAFLD

Obesity-induced fatty liver predisposes to non-alcoholic steatohepatitis (NASH), which has no approved pharmacotherapy, making it the fastest growing indication for liver transplantation. Fatty liver develops in part due to excess hepatic de novo lipogenesis (DNL), an insulin-stimulated cell-autonomous synthesis of fatty acids, a conundrum as obesity is commonly associated with insulin resistance and glucose intolerance. The serine/threonine phosphatase PHLPP2 terminates insulin action in the liver by dephosphorylating Akt Ser473 to repress DNL but not gluconeogenic pathways. Thus, BXD strains with lowest Phlpp2 expression and liver-specific PHLPP2 knockout (L-PHLPP2) mice show normal initiation of insulin signaling and intact glucose tolerance, but prolonged insulin action and hepatic steatosis. In obesity, endogenous PHLPP2 is degraded—to understand this regulation, we performed LC-MS/MS which identified glucagon/PKA-dependent PHLPP2 phosphorylations at Ser1119 and Ser1210, which recruit the adaptor KCTD17 and ubiquitin E3 ligase Cullin3. Hepatic Kctd17 expression is increased in murine obesity, and KCTD17 is correlated with excess hepatic fat in patients. Knockdown of hepatic Kctd17 in obese mice prevents PHLPP2 degradation, allowing normal repression of DNL. These results demonstrate the first direct signaling interaction between insulin and glucagon pathways, and suggest that modulators of the PHLPP2/KCTD17 axis can reverse obesity-induced hepatic steatosis to treat NASH.

### KCTD17 Targets PHLPP2 for Degradation to Induce NAFLD

The liver integrates nutrient state with the need to preserve glucose and lipids in a narrow physiologic range, with assistance from hormonal inputs (Pajvani and Accili, 2015). In normal physiology, fasting triggers the release of glucagon from pancreatic α-cells, which stimulates gluconeogenesis and glycogenolysis to maintain normoglycemia (Jelinek et al. 1993). In the postprandial state, pancreatic β-cells dial up insulin levels to block hepatic glucose production (Summers and Birnbaum, 1997; Puigserver et al., 2003; Petersen et al., 1998) and utilize excess glucose for long-term energy storage in the liver in the form of glycogen, and indirectly, triglycerides (Saltiel et al. 2001).

The elegance of this system is undermined by the chronic overnutrition associated with obesity, which leads to insulin resistance and an inability to restrain hepatic glucose production and the fasting hyperglycemia of Type 2 Diabetes (T2D). Nonetheless, compensatory hyperinsulinemia is apparently sufficient to activate sterol regulatory element-binding protein 1c (Srebp1c)-mediated lipogenesis (Shimomura et al., 2000; Matsumoto et al., 2006), which contributes to the excess hepatic triglyceride (TG) accumulation that defines Non-Alcoholic Fatty Liver Disease (NAFLD) (Donnelly et al., 2005). The mechanism underlying this “selective” insulin resistance remains under debate, but we and others have shown that insulin signaling diverges at the critical molecular node, Akt, to activate mTORC1-dependent and -independent lipogenic pathways (Shi et al., 2010; Yecies et al., 2011; Kim et al., 2016). But insulin doesn't act alone—the lipogenic program is under complex control by nutrient state and other hormones. Of note in this cadre is glucagon (Kersten et al., 2001), postprandial plasma levels of which remain inappropriately high in T2D patients (Butler et al., 1991; Dunning et al., 2007; Ali and Drucker, 2009). The interplay between insulin and glucagon pathways is further highlighted by the unique shared phenotype of reduced hepatic and plasma TG in liver-specific insulin receptor knockout (Biddinger et al., 2008) and glucagon receptor knockout mice (Conarello et al., 2007), but the potential role of glucagon in the selective insulin resistance seen in obesity has not been explored.

Pleckstrin homology (PH) domain leucine-rich repeat protein phosphatases (PHLPPs) have been shown to dephosphorylate the key regulatory Ser473 residue of Akt, thereby terminating growth factor-stimulated Akt activity and suppress tumor growth in cancer models (Gao et al., 2005; Grzechnik and Newton, 2016; Newton and Trotman, 2014). Recent work has demonstrated that PHLPPs, specifically the PHLPP2 isoform, similarly terminates insulin action in obesity (Kim et al., 2016). It was observed that diet-induced (HFD-feeding) or genetic (leptin-signaling deficient ob/ob or db/db mice) mouse models of obesity have lower PHLPP2 protein levels, which renders these mice unable to terminate insulin signaling, ultimately resulting in increased DNL and fatty liver (Kim et al., 2016). Consistently, exogenous “rescue” of hepatic PHLPP2 levels in obese mice reduced inappropriately elevated Akt Ser473 phosphorylation in the “late” refed state, and reversed fatty liver, but surprisingly, did not affect glucose tolerance. These data suggested that PHLPP2 is necessary to prevent insulin-potentiated DNL, but does not affect early post-prandial events such as insulin/Akt repression of FoxO-dependent hepatic gluconeogenesis in the bifurcation model of hepatic insulin signaling (Shi et al., 2010).

Several important questions remained from these studies, however—1) is chronic reduction of hepatic PHLPP2 levels sufficient to induce hepatic steatosis; 2) how is hepatic PHLPP2 lost in obesity, as Phlpp2 mRNA is unaffected in HFD-fed and genetic mouse models; and, 3) whether these data may have clinical and/or therapeutic implications. Here, we generate and characterize hepatocyte-specific PHLPP2 knockout (L-PHLPP2) mice, which show prolonged insulin-stimulated Akt Ser473 phosphorylation and excess DNL and fatty liver. To understand the endogenous regulatory machinery that mediates PHLPP2 degradation in obese liver, we performed sequential LC/MS-MS-based screens, which identified novel glucagon/PKA-mediated PHLPP2 phosphorylation at Ser1119 and Ser1210, which augments PHLPP2 interaction with KCTD17, an adaptor protein for Cul3-RING ubiquitin ligases (Kasahara et al., 2014). Interestingly, KCTD17 expression is increased in livers from obese mice and patients with NASH, linking glucagon-induced PHLPP2 phosphorylation with degradation. Thus, knockdown of KCTD17 in obese mice increases endogenous PHLPP2 and ameliorates obesity-induced fatty liver, revealing a novel strategy in the treatment of obesity-mediated NAFLD/NASH.

PHLPP2 Loss-of-Function Induces Fatty Liver but does not Affect Glucose Tolerance

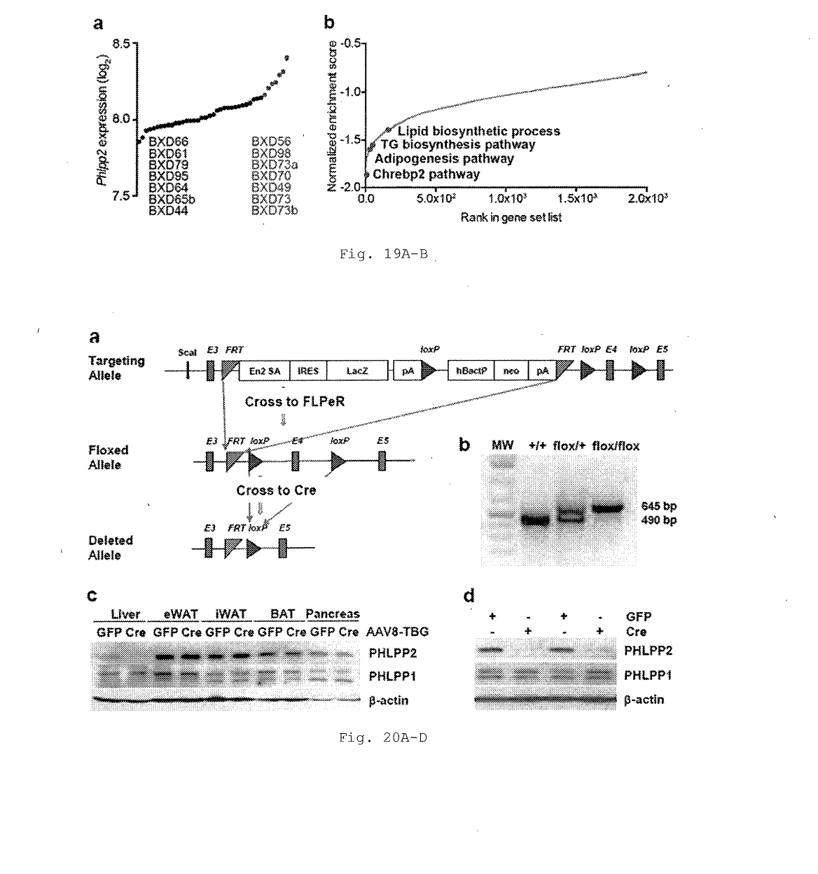

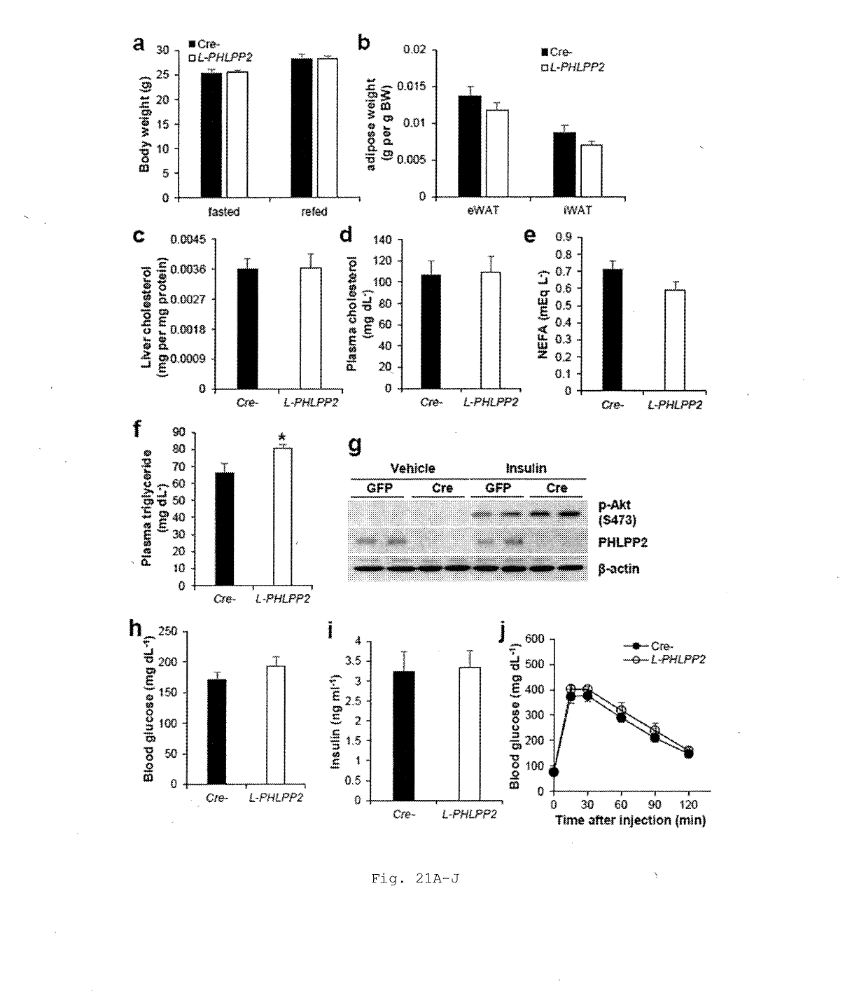

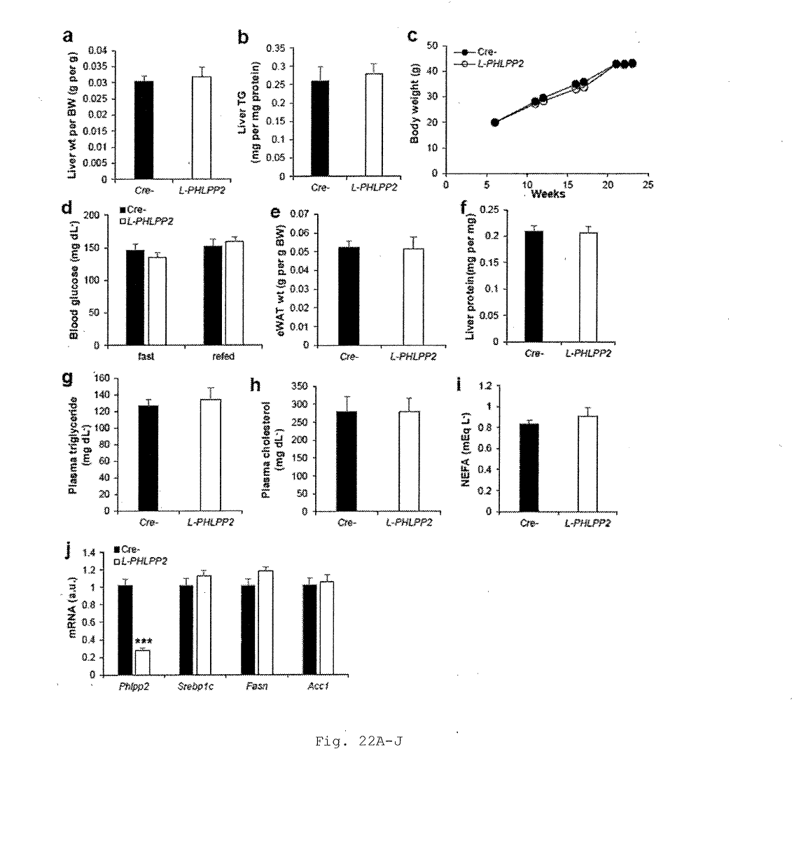

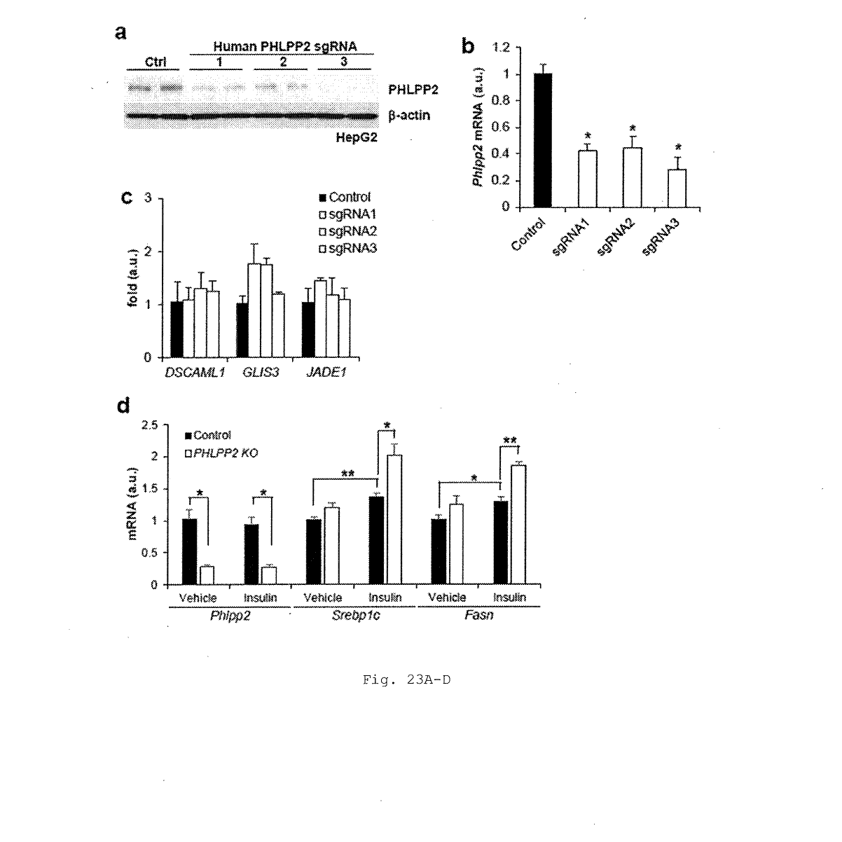

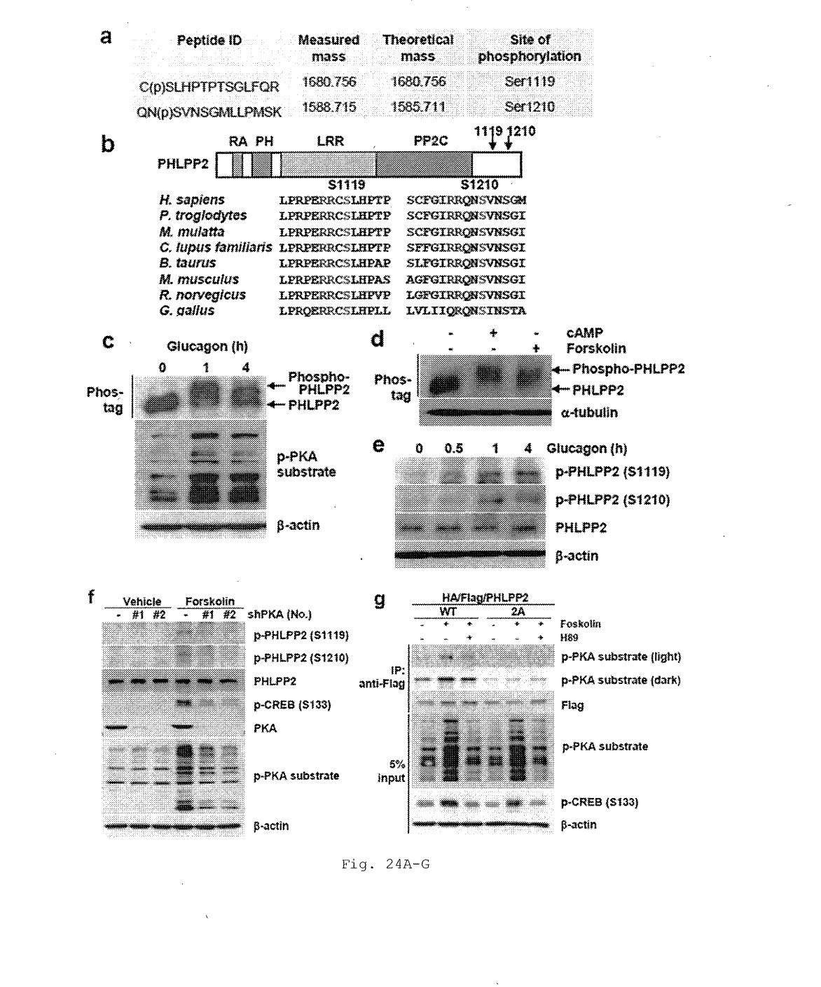

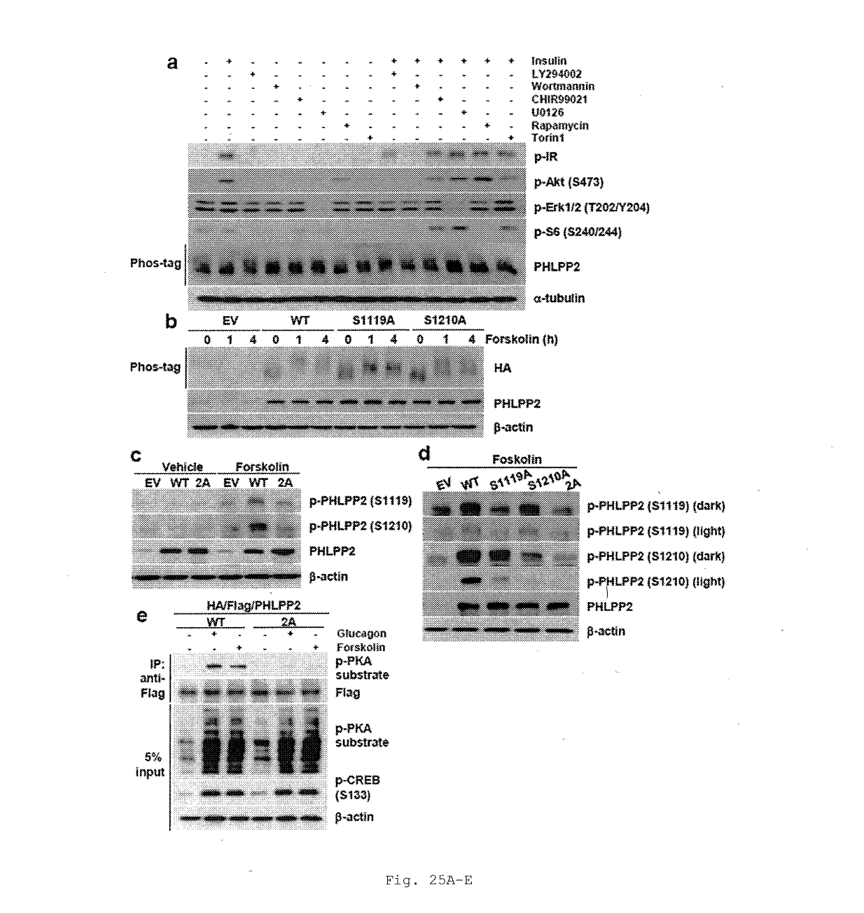

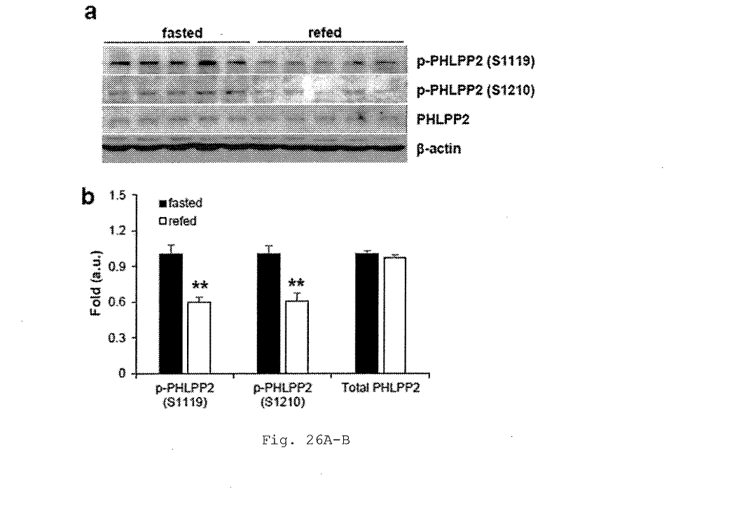

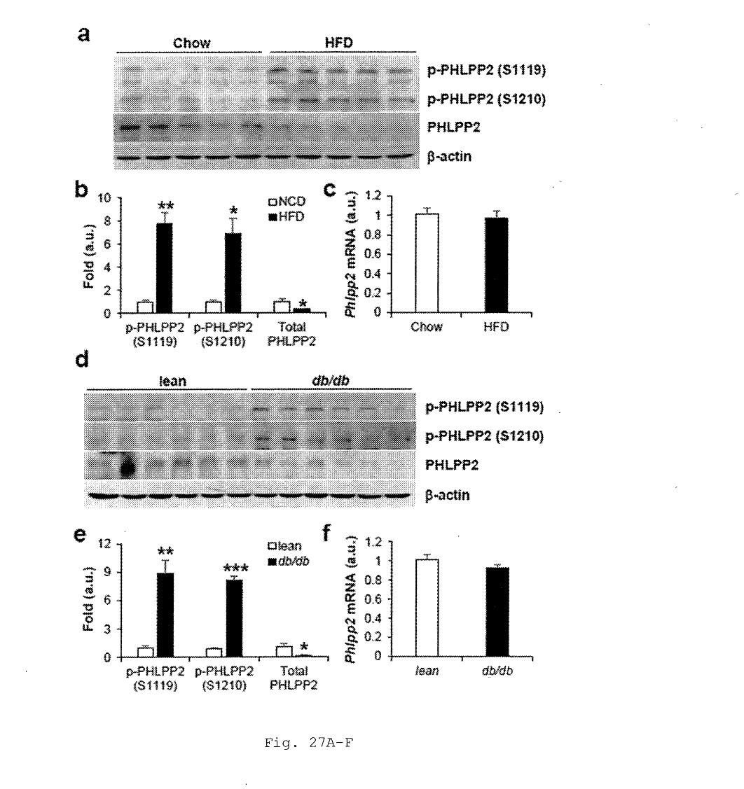

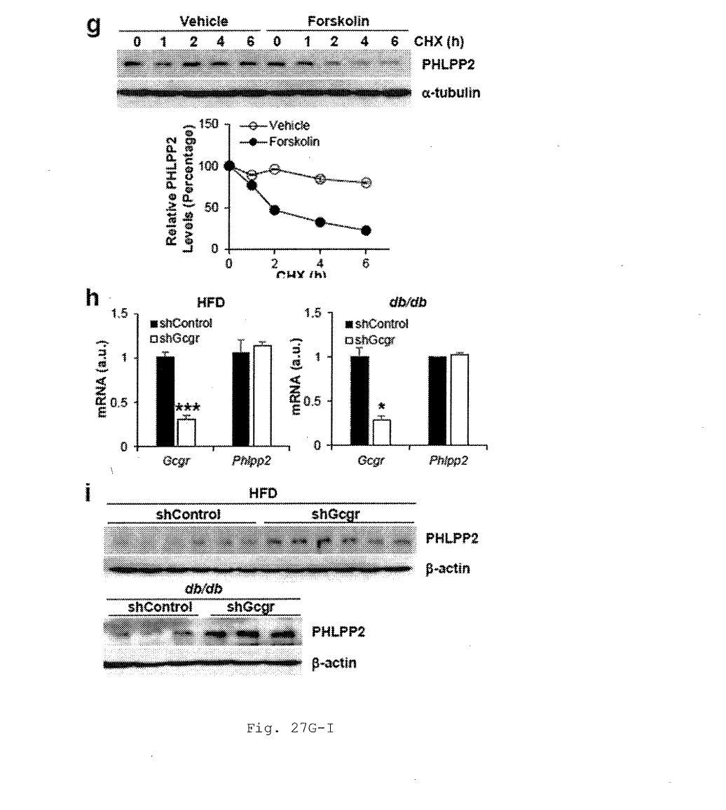

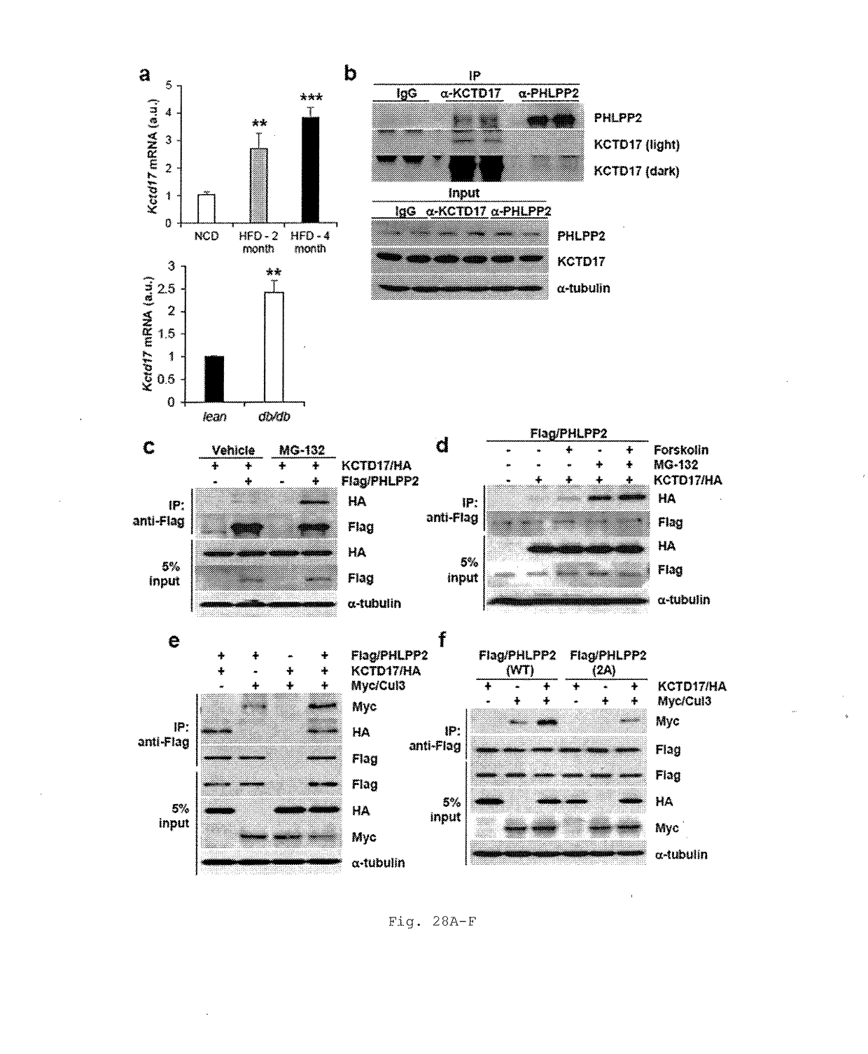

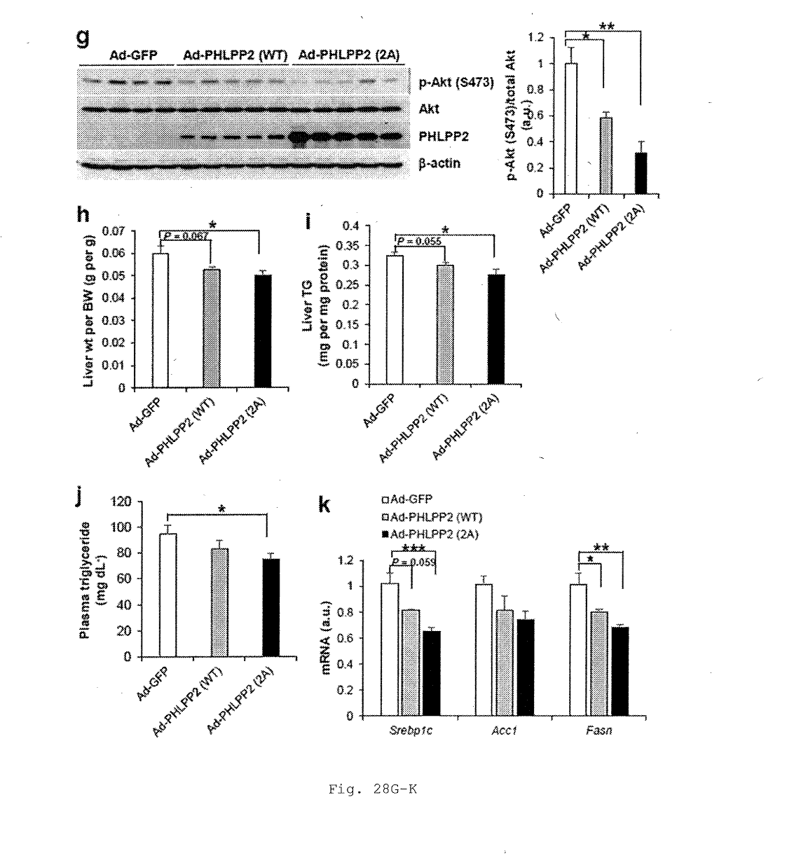

To explore endogenous differences in hepatic PHLPP2 levels and relationship to metabolism, 41 strains of genetically diverse BXD mice descendant from sequential C57BL/6J and DBA/2J crosses were examined—BXD mice have ^{˜}5 million diverged sequence variants and represents, at present, the largest and best-characterized mouse genetic reference population (Williams et al., 2016). A significant, inverse correlation was found between Phlpp2 expression and liver TG across these strains (FIG. 18a). As a first step to understand the mechanism underlying this inverse correlation, gene set enrichment analysis (GSEA) was performed using hepatic transcriptomes of 14 BXD strains showing highest and lowest expression of Phlpp2 (FIG. 19a). In this analysis, that Phlpp2 was found to be negatively correlated with lipogenic gene sets [Chrebp2 (NOM p<0.001), adipogenesis (NOM p<0.02), triglyceride biosynthesis (NOM p<0.02) and lipid biosynthesis (NOM p<0.02)] which all contain Srebp1 and its target lipogenic genes (FIG. 18b, c and FIG. 19b). Consistent with our observation that PHLPP2 overexpression does not affect glucose tolerance (Kim et al., 2016), no correlation was observed between Phlpp2 and gene sets associated with hepatic glucose production, or plasma levels of glucose (Spearman rho=0.11, p=0.567), insulin (Spearman rho=−0.11, p=0.502) and HOMA-IR (Spearman rho=0.10, p=0.553). These data show that across a genetically diverse mouse population, higher hepatic Phlpp2 expression is associated with lower lipid biosynthesis and may distinguish insulin-mediated functions in hepatic glucose and lipid metabolism.

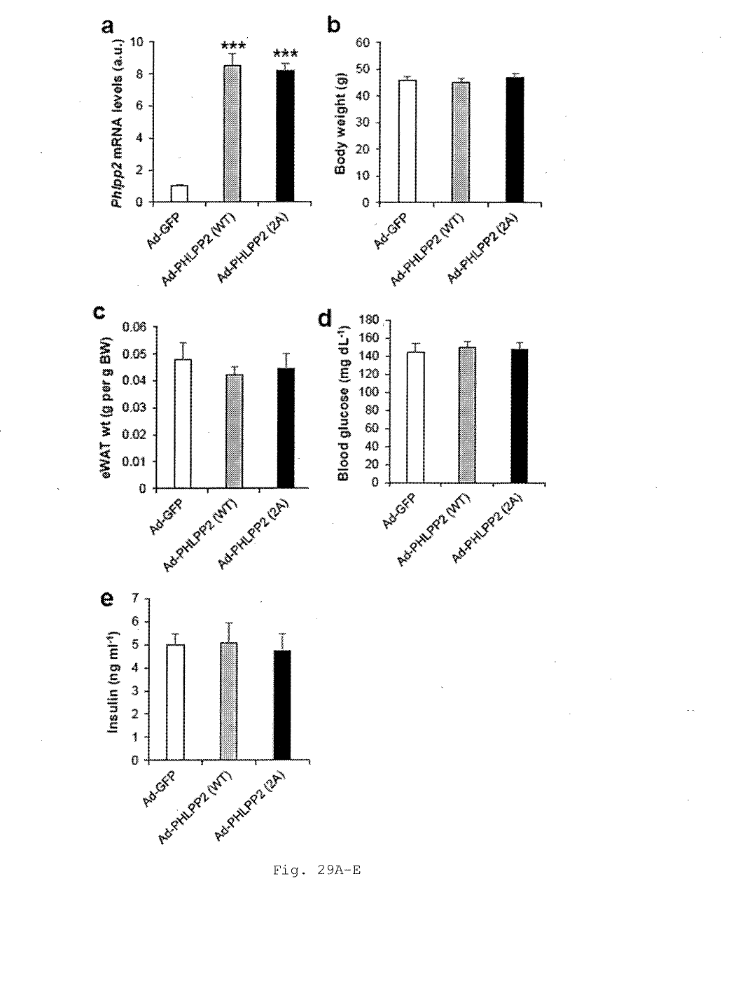

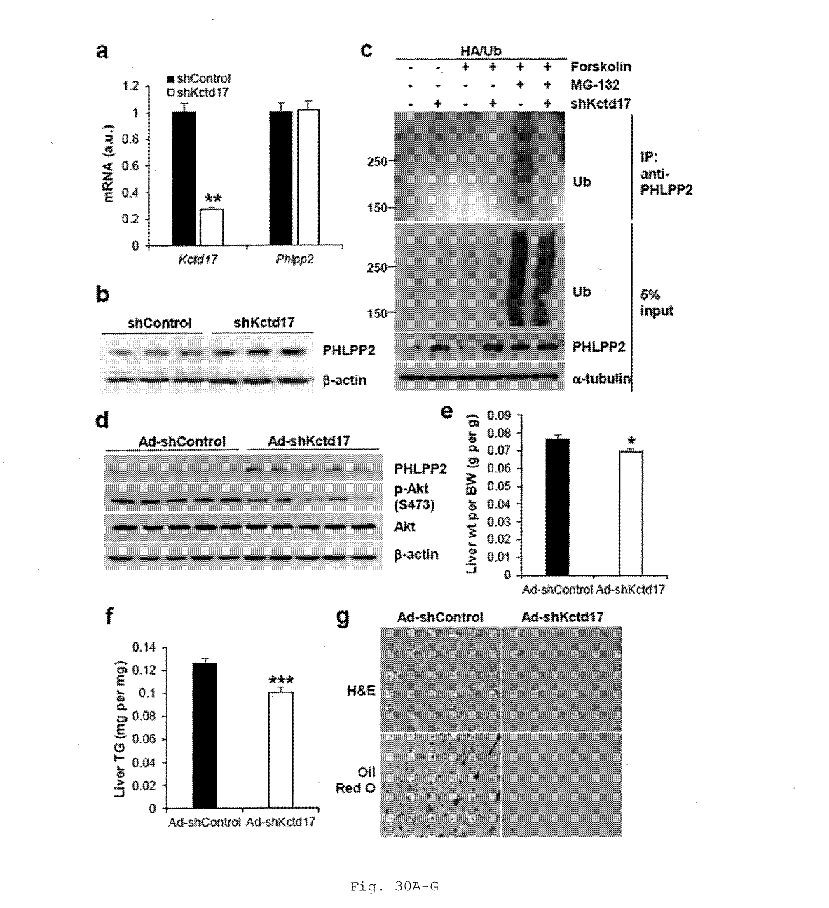

To mimic the pathologic loss of hepatic PHLPP2 seen in obese mice (Lim et al., 2016), which may mirror endogenous differences in BXD strains, we generated homozygous PHLPP2^{flox/flox} mice (FIG. 20a, b) and transduced with AAV8-TBG-Cre to produce hepatocyte-specific PHLPP2 knockout (L-PHLPP2) mice. Livers from L-PHLPP2 mice, or adenoviral Cre-mediated deletion of loxP-flanked PHLPP2 in primary hepatocytes, showed >80% PHLPP2 deletion without compensatory increase of the related isoform, PHLPP1 (FIG. 20c, d). PHLPP2^{flox/flox} mice develop normally, and post-AAV transduction, L-PHLPP2 mice show comparable body weight, adiposity and cholesterol/non-esterified fatty acid (NEFA) as control PHLPP2^{flox/flox} mice transduced with AAV8-TBG-GFP (FIG. 21a-e). Nevertheless, at sacrifice, L-PHLPP2 mice show increased liver weight and TG, with associated mild hypertriglyceridemia (FIG. 18d, e and FIG. 21f). Livers from L-PHLPP2 mice show higher Akt Ser473 phosphorylation in “late” refeeding, similar to Ad-Cre-transduced PHLPP2^{flox/flox} hepatocytes after prolonged insulin stimulation in vitro (FIG. 18f and FIG. 21g). Prolonged activation of hepatic insulin signaling through Akt thus leads to higher expression of Srebp1c and its canonical targets (i.e., Fasn), but does not affect gluconeogenic gene expression (FIG. 18g). Consistently, we observed no differences in fasting or refed blood glucose, plasma insulin or glucose tolerance (FIG. 19h-j).

Next, it was hypothesized that high-fat diet (HFD)-feeding to induce obesity and thus loss of endogenous PHLPP2 (Kim et al., 2016) would recapitulate the relative harm seen in chow-fed L-PHLPP2 mice, and thus negate differences between L-PHLPP2 and control mice. Indeed, HFD-fed L-PHLPP2 mice showed unchanged liver weight and TG from Cre-controls (FIG. 22a, b), and in fact no discernible metabolic phenotype (FIG. 22c-i) as well as unchanged lipogenic gene expression (FIG. 22j). In combination, these data would suggest that early postprandial insulin action, necessary for nuclear FoxO exclusion and cessation of gluconeogenesis (Haeusler et al., 2014) is unimpeded, but that late postprandial insulin action on lipogenesis is heightened by either endogenous (HFD feeding) or artificial (L-PHLPP2 mice) loss of PHLPP2.

To confirm that PHLPP2 terminates insulin-Akt signaling in a cell-autonomous manner, we generated PHLPP2 knockout (KO) hepatoma cells using CRISPR/Cas9 (FIG. 23a-c). PHLPP2 KO cells showed similar insulin-induced Akt phosphorylation as compared to control cells, but delayed Akt dephosphorylation in the “chase” period after the insulin pulse, which was normalized by PHLPP2 reconstitution (FIG. 18h). Similar to data from L-PHLPP2 mice, PHLPP2 KO cells show augmented insulin-stimulated Srebp1c and Fasn as compared with control cells (FIG. 23d). These data prove that PHLPP2 is required for termination of insulin/Akt signaling, which prevents excess Srebp1c-mediated DNL and hepatic lipid accumulation.

PHLPP2 is phosphorylated on Ser1119 and Ser1210 by glucagon/PKA action Obese mice show lower hepatic PHLPP2 levels, but unchanged Phlpp2 gene expression (Kim et al., 2016). Based on this finding, we hypothesized that PHLPP2 levels may be affected by hormone or nutrient-regulated post-translational modifications (PTMs). To test this hypothesis, we performed liquid chromatography-tandem mass spectrometry (LC-MS/MS) analysis in immunoprecipitated PHLPP2 from livers of C57BL/6 mice transduced with HA/Flag-tagged PHLPP2, and found 2 phosphopeptides—RCpSLHPTPTSGLFQR (Ser1119) and RQNpSVNSGMLLPMSK (Ser1210)—at evolutionarily-conserved PKA consensus [RRX(S/T)] sites (FIG. 24a, b). As glucagon is the major driver of PKA activity in liver (McKnight et al. 1991), we next tested whether glucagon can induce PHLPP2 phosphorylation. Indeed, using Phos-tag-based electrophoresis, we found that PHLPP2 is rapidly phosphorylated in primary hepatocytes exposed to glucagon (FIG. 24c), similar to treatment with potent PKA activators such as cAMP or forskolin (FIG. 24d), but not after activation of other hormone/nutrient signaling (i.e. insulin/PI3K, MEK, GSK or mTOR) pathways (FIG. 25a). We next used site-directed mutagenesis to create Ser→Ala mutations at Ser1119 and Ser1210, but found that mutation of either site did not fully abolish forskolin-induced PHLPP2 phosphorylation (FIG. 25b). We next generated polyclonal antibodies to both p-PHLPP2 (Ser1119) and p-PHLPP2 (Ser1210), and confirmed specificity of these antibodies in primary hepatocytes transfected with WT, single (S1119A or S1210A) or double (S1119A/S1210A) PHLPP2 Ser→Ala mutants (FIG. 25c, d). Using these novel reagents, we found that endogenous PHLPP2 phosphorylation at Ser1119 and Ser1210 is increased with acute glucagon treatment of primary hepatocytes (FIG. 24e). As such, acute forskolin-induced phosphorylation at both Ser1119 and Ser1210 was reduced by knockdown of PKA signaling (FIG. 24f). Thus, mutation of both Serine sites (2A) led to near-absent PHLPP2 phosphorylation in primary hepatocytes treated with glucagon or forskolin (FIG. 25e), recapitulating treatment with the PKA inhibitor H-89 (FIG. 24g). In sum, these data prove that glucagon-induced PKA activity phosphorylates hepatocyte PHLPP2 at Ser1119 and Ser1210.

### Glucagon-Mediated PHLPP2 Phosphorylation Reduces its Stability in Obesity

Glucagon is acutely increased in the fasted animal (Jelinek et al., 1993). Thus, it was find that hepatic PHLPP2 phosphorylation at both Ser1119 and Ser1210 is dynamically regulated by the fasting-refeeding transition in vivo (FIG. 26a, b). Obesity and T2D are associated with persistent hyperglucagonemia, which may regulate hepatic lipogenesis (Horton et al., 2002; Strable and Ntambi, 2010; Perry et al., 2014). It was hypothesized that obesity may provoke “chronic” PHLPP2 phosphorylation. Indeed, markedly increased hepatic PHLPP2 phosphorylation and a simultaneous reduction in total PHLPP2 protein in HFD-fed were observed (FIG. 27a-c) and in leptin-receptor (db/db) deficient (FIG. 27d-f) mice, but strikingly, no change in Phlpp2 mRNA. These results suggested that PHLLP2 phosphorylation may directly affect its stability; to test this, cells were treated with cycloheximide to inhibit new protein translation, and markedly decreased PHLPP2 protein was found in the presence of forskolin (FIG. 27g). Next, it was examined whether inhibition of hepatic glucagon signaling, by means of AAV8 encoding shRNA to the glucagon receptor (AAV8-shGcgr), could prevent loss of PHLPP2 seen in HFD-fed or db/db mice. Indeed, as compared to AAV8-shControl-transduced mice, AAV8-shGcgr transduction robustly increase hepatic PHLPP2 without affecting Phlpp2 mRNA (FIG. 27h, i). Similar data were obtained with use of glucagon receptor antagonists (GRAs) in primary hepatocytes. It was concluded that glucagon-induced hepatic PHLPP2 phosphorylation affects PHLPP2 stability.

### KCTD17 Binds PHLPP2 to Induce its Degradation in Obese Liver

Although glucagon induces PHLPP2 phosphorylation in both fasting and obesity, comparing FIGS. 27b and 27e with FIG. 26b, only obesity reduced PHLPP2 protein levels. These data imply that PHLPP2 phosphorylation may be necessary but cannot be sufficient to induce putative PHLPP2 degradation, and that a “second hit” beyond hyperglucagonemia is necessary for the post-transcriptional loss of PHLPP2 seen in obese liver. To identify the regulatory mechanisms of glucagon-induced PHLPP2 degradation, LC/MS-MS was performed with adjusted purification conditions to isolate PHLPP2-binding proteins. I resultant list of PHLPP2 interactors for potential regulatory roles in protein stability and/or hepatic metabolism was sequentially queried, and for altered expression in obese liver. From this analysis, KCTD17 (potassium channel tetramerization domain containing 17) was identified as a novel PHLPP2-associated protein. Despite its name, KCTD17 has been shown to act as a substrate-adaptor for Cul3-RING ubiquitin ligases (CRL3s) to regulate proteosomal degradation of specific targets (Kasahara et al., 2014; Inaba et al., 2016; Ji et al., 2016). First, to explore its potential role in metabolic processes, hepatic Kctd17 expression was examined and found to be robustly increased in HFD-fed and db/db mice (FIG. 28a). Next, to confirm our LC-MS/MS results, we immunopreciptated endogenous KCTD17 in livers from HFD-fed mice using anti-PHLPP2 antibody, and vice versa (FIG. 28b). KCTD17-PHLPP2 interaction was increased by proteosomal inhibitors (FIG. 28c) and synergistically with forskolin co-treatment (FIG. 28d), suggesting that glucagon-mediated PHLPP2 phosphorylation and degradation are linked by KCTD17. Indeed, KCTD17 increased PHLPP2-Cul3 interaction (FIG. 28e), but far less efficiently facilitated the interaction of Cul3 with the nonphosphorylatable PHLPP2-2A mutant (FIG. 28f). We next hypothesized that the PHLPP2-2A mutant may be relatively resistant to degradation—to test this, we transduced HFD-fed mice with adenovirus encoding GFP, PHLPP2-WT or PHLPP2-2A. Despite similar mRNA induction as compared to Ad-GFP-transduced mice (FIG. 29a), PHLPP2-2A induced far greater increase in hepatic PHLPP2 levels than PHLPP2-WT, and thus greater decrease in refed hepatic Akt (Ser473) phosphorylation (FIG. 28g). Thus, we found a gradated reduction in liver weight, liver TG and lipogenic gene expression as well as plasma TG from control→PHLPP2-WT→PHLPP2-2A-transduced mice (FIG. 28h-k), despite similar body weight/adiposity and glucose homeostasis among the groups (FIG. 29b-e). In sum, these data show that glucagon-induced phosphorylation and KCTD17-recruitment of Cul3 synergistically target PHLPP2 for degradation, which then increases DNL to provoke hepatic steatosis.

Knockdown of KCTD17 prevents diet-induced hepatic steatosis

It was next predicted that KCTD17 may be sufficient to protect PHLPP2 from degradation. shRNA directed against Kctd17 was produced, and it was found that Kctd17 knockdown increased PHLPP2 protein but not Phlpp2 mRNA expression in primary hepatocytes (FIG. 30a, b) and in hepatoma cell lines (FIG. 31a, b). As hypothesized, acute Kctd17 knockdown strongly reduced PHLPP2 ubiquitination (FIG. 78c), to completely block insulin-mediated induction of lipogenic genes (FIG. 31c). Based on these findings, it was predicted that reducing obesity-mediated increase of hepatic Kctd17 would reproduce the lower liver TG seen with Ad-PHLPP2 or -2A-transduction, by “rescuing” lower PHLPP2 levels in obese mice. Consistent with in vitro data, Ad-shKctd17-transduced mice showed increased hepatic PHLPP2 protein levels and concomitantly reduced late refed Akt Ser473 phosphorylation (FIG. 30d), without change in Phlpp2 expression (FIG. 31d). Remarkably, this acute reduction in Kctd17 was sufficient to lower liver weight and TG relative to Ad-shControl mice (FIG. 30e-g), without confounding changes in body weight/adiposity (FIG. 31e, f). Further, similar to Ad-PHLPP2 or -2A-transduced mice, we observed lower lipogenic but unchanged gluconeogenic gene expression in Ad-shKctd17-transduced livers (FIG. 30h).

KCTD17 Correlates with Lipogenic Gene Expression and Hepatic Steatosis in NASH

These data show that KCTD17 regulates hepatic steatosis, at least in part by mediating PHLPP2 degradation. To begin to understand the implication of these findings for human disease, liver KCTD17 expression was analyzed in patients undergoing liver biopsy for suspected NASH (Table A), and a significant positive correlation was found between KCTD17 and hepatic steatosis (FIG. 32a).

TABLE A
Demographic, clinical and histological features
in a cross-sectional liver biopsy cohort.

| | Liver biopsy cohort (n = 158) | |
|---|---|---|
| Age, years | 45 ± 10 | |
| Sex, female | 95 | (60) |
| BMI, Kg/m² | 37.8 ± 8.5 | |
| Type 2 Diabetes, yes | 41 | (26) |
| ALT, IU/I | 28 | [18-157] |
| AST, IU/I | 22 | [17-33] |
| PNPLA3, 148M/M | 25 | (16) |
| Histological steatosis grade | | |
| 0 | 17 | (11) |
| 1 | 50 | (32) |
| 2 | 49 | (31) |
| 3 | 42 | (27) |
| Definite NASH, yes | 31 | (19) |

Data represent means of ±SD, median [interquartile range], or number (%).

This positive correlation was the primary driver for a similar positive correlation between KCTD17 and NAFLD Activity Score (NAS) (FIG. 32b), as higher hepatic KCTD17 expression was no longer associated with increased NAS once adjusted for steatosis in multivariate regression analysis (Table B).

TABLE B
Predictors of hepatic KCTD17 expression in
the cross-sectional liver biopsy cohort.

| | Univariate analysis | | | Multivariate analysis | | |
|---|---|---|---|---|---|---|
| | | | P | | | P |
| | Beta | SE | value | Beta | SE | value |
| Age, years | +0.03 | 0.01 | 0.001 | +0.02 | 0.01 | 0.012 |
| Sex, Female | −0.24 | 0.09 | 0.008 | −0.07 | 0.10 | 0.46 |
| BMI, Kg/m² | −0.03 | 0.01 | 0.011 | −0.02 | 0.01 | 0.061 |
| Type 2 diabetes, yes | +0.33 | 0.10 | 0.001 | +0.12 | 0.11 | 0.25 |
| PNPLA3 148M, alleles | −0.08 | 0.13 | 0.55 | −0.19 | 0.12 | 0.13 |
| Steatosis grade | +0.37 | 0.09 | <0.0001 | +0.28 | 0.11 | 0.008 |
| Histological activity | +0.31 | 0.09 | 0.0008 | +0.02 | 0.11 | 0.87 |
| (Necroinflammation + | | | | | | |
| ballooning) | | | | | | |

SE: standard error.
Comparisons were made by fitting data to generalized linear models, unadjusted (univariate analyses), or considering as independent variables: age, sex, BMI, type 2 diabetes, PNPLA3 I148M alleles, histological steatosis grade, and activity. Hepatic KCTD17 mRNA levels were normalized for β-actin and log transformed before analyses to ensure a normal distribution.

These data suggest that the association with NASH observed at univariate analysis may be secondary to more severe steatosis. Interestingly, KCTD17 expression was not associated with the PNPLA3 I148M variant, the predominant determinant of hepatic fat accumulation in humans, which is consistent with our mouse data showing increased Kctd17 is causative and not a simple consequence of hepatic steatosis.

These data derived from a cross-sectional cohort, which may limit its generalizability. To determine if KCTD17 expression co-varies with NAFLD/NASH severity, we analyzed liver transcriptomes in patients with NASH before and after bariatric surgery, as well as normal controls (Ahrens et al., 2013), and found a striking co-regulation of KCTD17 and lipogenic gene sets in patients with NASH (FIG. 32c). In paired analysis, KCTD17 expression was significantly higher in patients with NASH as compared to normal controls, and significantly reduced after bariatric surgery (FIG. 32d). Thus, KCTD17 showed a strong positive correlation with lipogenic and inflammatory gene expression (FIG. 32e and FIG. 33a), which closely clustered as a gene network (FIG. 33b), but weaker/indirect associations with other pathways associated with NASH, such as β-oxidation, ER stress and fatty acid esterification (FIG. 33b). In combination with data from L-PHLPP2 and the similar phenotypes of Ad-PHLPP2-2A and Ad-shKctd17 mice, these results establish the functional significance of glucagon-mediated KCTD17/Cul3 degradation of PHLPP2 in regulation of hepatic lipid accumulation.

### DISCUSSION

U.S. Nonprovisional application Ser. No. 15/143,305, filed Apr. 29, 2016, describes Raptor exists in both the mTORC1-bound and -independent (“free”) state in liver, and that free Raptor levels decline markedly in aging and obesity. Additionally, U.S. Nonprovisional application Ser. No. 15/143,305, filed Apr. 29, 2016, describes “free Raptor” as a regulator of hepatic PHLPP2, which promotes Akt Ser473 dephosphorylation and negatively regulates hepatic de novo lipogenesis without affecting hepatic glucose production. Accordingly, U.S. Nonprovisional application Ser. No. 15/143,305, filed Apr. 29, 2016, describes a role of free Raptor and PHLPP2 as a novel therapeutic targets for non-alcoholic fatty liver disease (NAFLD).

Furthermore, U.S. Nonprovisional application Ser. No. 15/143,305, filed Apr. 29, 2016, identifies glucagon induces phosphorylation of PHLPP2 at Ser1119 and Ser1210, and inhibitors of glucagon signaling increase the pathologically lower PHLPP2 levels in the obese state to effectively reduce hepatic and plasma triglyceride levels. U.S. Nonprovisional application Ser. No. 15/143,305, filed Apr. 29, 2016, also identifies KCTD17 is a novel PHLPP2 interacting protein, which may regulate PHLPP2 degradation in a phosphorylation/proteasome-dependent manner. The aforementioned disclosures of U.S. Nonprovisional application Ser. No. 15/143,305, filed Apr. 29, 2016, are hereby incorporated by reference into this disclosure.

Obesity causes insulin resistance and compensatory hyperinsulinemia, which then activates both mTORC1-dependent and -independent pathways to induce DNL (Shi et al., 2010; Yecies et al., 2011; Kim et al. 2016). Less appreciated is the possible effects of obesity-induced hyperglucagonemia (Lefebvre et al., 1995), traditionally thought to antagonize insulin's beneficial effects on glucose homeostasis (Conarello et al., 2007). Our unbiased screen has revealed the first clear intersection between insulin and glucagon signaling pathways on DNL, through PHLPP2 (FIG. 32f). PHLPP2, previously shown by our group and others to dephosphorylate Akt at Ser473 to terminate insulin action, is rapidly phosphorylated by glucagon/PKA action to trigger PHLPP2 degradation. In fact, expression of a glucagon-resistant nonphosphorylatable PHLPP2 mutant prevents obesity-induced hyperinsulinemia from inducing DNL, and thus reverses fatty liver, echoing results from various glucagon antagonists in preclinical development (Gelling et al., 2003; Sloop et al., 2004; Sorensen et al., 2006). These results may explain the molecular mechanism underlying these observations, but also imply that other unrecognized molecular bridges between two critical hormone-regulated pathways to regulate DNL may exist. This may have implications even beyond metabolism, as DNL has emerged as a promising pathway for antineoplastic therapy.

L-PHLPP2 mice was found to show fatty liver, but normal glucose homeostasis. While this may appear paradoxical on face, this finding is actually quite consistent with normal signal transduction through InsR→PI3K→Akt observed in response to feeding in L-PHLPP2 mice, or following an insulin “pulse” in PHLPP2 KO cells. In the absence of PHLPP2, however, prolonged Akt phosphorylation was observed during the “chase” period, and downstream increase in DNL due to a failure to terminate the insulin action. Conversely, adenovirus-mediated overexpression of PHLPP2 reduces liver TG, but also does not affect glucose homeostasis (Kim et al., 2016). These data suggest the bifurcation model of insulin signaling (Shi et al., 2010) may still explain the “selective” insulin resistance seen in the obese liver, but show that the kinetics of downstream signaling is as important as overall insulin levels—early post-prandial Akt activation is necessary to reduce hepatic glucose output, but prolonged Akt activity only increases DNL. These data suggest that this smoldering, chronic hepatic insulin signaling is partially responsible for increased liver TG in obesity without accompanying glycemic benefit.

LC/MS-MS screen identified PHLPP2 phosphorylations at both Ser1119 and Ser1210 in a previously undefined but evolutionarily-conserved domain of PHLPP2. Glucagon/PKA-mediated PHLPP2 phosphorylation at these sites is necessary but not sufficient to induce its degradation. Our second LC/MS-MS screen pointed to one potential mechanism for this by identifying KCTD17 as a novel PHLPP2-interacting protein. It was shown that the obesity-mediated increase in hepatic Kctd17 expression is necessary to link PHLPP2 phosphorylation with degradation, as Kctd17 knockdown in obese mice prevented PHLPP2 degradation, normalized Akt signaling and reduced liver TG. Studies to determine the molecular mechanism underlying increased Kctd17 expression in obese liver are ongoing. KCTD17 expression seems to preferentially predict steatosis. Additionally, KCTD17 may mediate “multiple hits” in NAFLD/NASH pathogenesis (Tilg and Moschen, 2010; Peverill et al., 2014; Buzzetti et al., 2016). Accordingly, further testing of KCTD17 effects in dietary mouse models of NASH (Wang et al., 2016) is needed.

In summary, obesity induces glucagon-mediated phosphorylation of PHLPP2, which targets its degradation by KCTD17/Cul3, which in turn increases insulin-mediated DNL and causes fatty liver. These data suggest that while glucagon receptor antagonists may have adverse effects (Kelly et a;/. 2015; Kazierad et al., 2016), inhibition of PHLPP2 phosphorylation, perhaps by dint of KCTD17 antagonism, may ameliorate obesity-induced fatty liver without affecting glucose homeostasis and stem the tide of liver disease in an increasingly obese population.

### Example 3

### Advantages of KCTD17 Inhibition

Hepatic de novo lipogenesis (DNL) is the hepatocyte conversion of 2-carbon precursors derived from metabolism of sugars into fatty acids. DNL is increased in and contributes the pathogenesis of non-alcoholic fatty liver disease (NAFLD) (Donnelly et al., 2005), the most common chronic liver disease and the fastest-growing risk factor for liver transplantation, and often associated with type 2 diabetes (T2D). One of the major stimulatory pathways for DNL is the sterol regulatory element binding protein 1c (SREBP1c)-mediated transcriptional regulation of enzymes which integral to fatty acid synthesis. SREBP1c in turn is activated by insulin signaling, via activation of the insulin receptor, and canonical signaling through the phosphoinositide-3 kinase (PI3K)/Akt pathway (Hegarty et al., 2005; Yellaturu et al., 2009). SREBP1c is also directly activated by the mammalian target of rapamycin complex 1 (mTORC1) (Ricoult and Manning, 2013), which in turn is also activated by insulin signaling, highlighting the intersection of hormones and nutrient to regulate DNL.

Accordingly, it was proposed that inhibition of these two pathways (insulin and mTORC1), or their final common mediator (SREBP1c) may reduce DNL, and thus ameliorate NAFLD. However, inhibition of liver insulin action by Akt deletion in mice has been shown to induce severe hyperglycemia, liver inflammation and hepatocellular carcinoma (HCC) (Lu et al., 2012; Wang et al., 2016). Further, use of the mTORC1 inhibitor, rapamycin, in clinical practice as an immunosuppressant often results in dyslipidemia (Wang et al., 2016), and chronic rapamycin treatment in rats causes insulin resistance and glucose intolerance, in part though inducing hepatic gluconeogenesis (Houde et al., 2010). Thus, inhibition of the upstream pathways (insulin and mTORC1) will have a poor risk/benefit ratio for chronic treatment of NAFLD. Similarly, investigators have determined that a small molecule inhibitor of SREBP1c, 75onalco, can ameliorate diet-induced fatty liver and insulin resistance (Tang et al., 2011), but in other studies, induces apoptosis through decreased mitochondrial membrane potential, activation of MAPK and modulation of NF-kB in cancer cell lines (Gupta et al., 2010). Thus, inhibition of all major upstream pathways that regulate DNL (insulin and mTORC1), and even more specific inhibition of the effector (via the molecular activation of SREBP1c) are likely to be plagued with side-effects precluding application for chronic use in patients with NAFLD. Thus, new molecular targets that inhibit DNL without these side effects of glucose intolerance and liver damage/cancer are sorely needed. KCTD17 inhibition should reduce DNL and thus liver fat, without these side effects, as it bypasses the need to fully inhibit insulin or mTORC1 signaling (and thus does not have glucose intolerance or liver inflammation/cancer as a side effect) and only modulates SREBP1c-induced DNL without full inhibition which should preclude off-target effects on cell death.

### Methods

### Bioinformatics

For BXD analyses, unbiased GSEA (http://www.broadinstitute.org/gsea) was performed using hepatic transcriptomes as previously described (Ryu et al., 2014; Subramanian et al., 2005). Briefly, hepatic Phlpp2 was assessed in BXD mouse genetic reference populations fed normal chow diet (GeneNetwork Accession No: GN432), and transcriptomes corresponding to the top and bottom sextiles subjected to GSEA.

All raw transcriptomic data related to human NASH samples are publicly available on Gene Expression Omnibus (GEO; www.ncbi.nlm.nih.gov/geo) under accession numbers (GSE60149 and GSE48452)(Williams et al., 2016; Ahrens et al., 2013). Heat maps were built using GENE-E (www.broadinstitute.org/cancer/software/GENE-E). The depth of shading at the correlation matrices (correlogram) indicates the magnitude of the correlation (Spearman's Rho). Correlogram and interaction network were generated using Rstudio (www.rstudio.com). Positive and negative correlations are represented in blue and red, respectively. For the interaction network, blue and red colored edge indicate positive and negative correlations, respectively. Only correlations with Spearman's Rho >0.5 or <−0.5 are displayed in the interaction network.

### Animals

Male, wild-type C57BL/6J (strain #662) and male leptin-deficient db/db (strain #642) mice were purchased from Jackson Labs. PHLPP2 floxed mice were generated using a targeting vector purchased from EUCOMM, which was modified, linearized and electroporated into embryonic stem (ES) cells. Antibiotic-resistant ES cell clones were microinjected into C57BL/6 blastocysts to obtain chimeric mice. Founders that exhibited germline transmission were bred with C57BL/6J mice expressing flp recombinase (strain #016226) to remove the neo cassette (PHLPP2^{flox/+}) were then backcrossed >5 generations into C57BL/6J background, then intercrossed to generate homozygous PHLPP2^{flox/flox} mice. L-PHLPP2 mice were created by transduction of male PHLPP2^{flox/flox} mice with AAV8-TBG-Cre (Penn Vector Core). L-PHLPP2 mice were fed standard chow (Purina Mills 5053) or high-fat diet (18.4% calories/carbohydrates, 21.3% calories/protein and 60.3% calories/fat derived from lard; Harlan Laboratories, TD.06414). All mice were housed 3-5 animals per cage, with a 12 h light/dark cycle, in a temperature-controlled environment. Number of animals used in experiments was chosen to ensure adequate power to detect experimental difference with alpha set to 0.05. All animal experiments were approved by the Columbia University Institutional Animal Care and Utilization Committee.

### CRISPR Generation of PHLPP2-Knockout (KO) Cells and Cell Cultures Studies

20 nucleotide single-guide RNA (sgRNA) sequences were designed using the CRISPR design tool at www.genome-engineering.org/crispr. sgRNAs were cloned into LentiCRISPRv2 from Addgene (plasmid #52961), and lentivirus encoding these sgRNAs was generated by co-transfection of 293T cells with the lentiviral vectors psPAX2 and pMD2.G. Viral supernatant was collected, and with polybrene (Sigma), applied to HepG2 or Hepa1c1c7 cells, which were selected with puromycin prior to expansion of single clones, screening by Western blot and genomic sequencing. Primary mouse hepatocytes were isolated as previously described (Kim et al., 2016; Pajvani et al., 2013).

### Chemicals

The following chemicals were used in this study: puromycin, N-ethylmaleimide (ThermoFisher), polybrene, insulin, cAMP, forskolin, glucagon, Trichostatin A (Sigma), H-89 (Merck) or Phos-tag reagent (Wako).

### Antibodies; Western Blots and Immunoprecipitation

Liver and cellular lysates were obtained by centrifugation as previously described (Kim et al., 2016). Immunoblots were conducted on 3-7 samples randomly chosen within each experimental cohort with antibodies against Akt (#9272), p-Akt (S473, #4060), p-Akt (T308, #13038), p-S6 (S240/244, #2215), S6 (#2217), p-(Ser/Thr) PKA substrate (#9621), PKA (#4782), DYKDDDDK-tag (#14793), HA-tag (#3724), myc-tag (#2276), and β-actin (#4970) from Cell Signaling; and PHLPP1 (A300-660A) and PHLPP2 (A300-661A) from Bethyl Laboratories, Inc. Antibodies directed to novel PHLPP2 phosphorylations (S1119 or S1210, which is identical to S1116 or 51207 in mouse PHLPP2) were generated by immunizing rabbits with synthetic 79onalcoh-specific peptides, verified by ELISA and affinity purified (Lifetein). Specificity of anti-PHLPP2 (S1119) and anti-PHLPP2 (S1210) were confirmed in PHLPP2 KO cells.

### Mass Spectrometry

PHLPP2 was purified from livers of male, wild-type C57BL/6J mice transduced with Ad-HA-FLAG-PHLPP2. To identify PHLPP2 PTMs, liver lysates were prepared in stringent lysis buffer [25 mM Tris-HCl (pH 7.5), 150 mM NaCl, 1 mM EDTA, 10% glycerol, 1% NP-40, 0.05% SDS, 1% sodium deoxycholate, 10 mM □-glycerophosphate, 10 mM sodium pyrophosphate, 50 mM sodium fluoride, 1.5 mM sodium orthovanadate, 30 □M Trichostatin A, 20 mM sodium butyrate, 20 mM nicotinamide, 25 mM N-ethylmaleimide and protease inhibitor cocktail (Pierce)], prior to immunoprecipitation with anti-FLAG antibody-conjugated M2 agarose (Sigma) and separation of the enriched proteins by SDS-PAGE. The gel was stained with Coomassie blue, and the band corresponding to PHLPP2 excised, followed by protein extraction, trypsin digestion (Balasubramanian et al., 2016) and analysis by nanoflow liquid chromatography-tandem mass spectrometry (LC-MS/MS) analysis. To examine PHLPP2 interacting proteins, liver lysates were extracted in less stringent lysis buffer [50 mM Tris-HCl (pH 7.5), 150 mM NaCl, 1 mM EDTA, 10% glycerol, 1% NP-40, 10 mM β-glycerophosphate, 10 mM sodium pyrophosphate, 50 mM sodium fluoride, 1.5 mM sodium orthovanadate, and protease inhibitor cocktail (Pierce)], immunoprecipitated with anti-Flag M2 agarose, and bound polypeptides eluted with FLAG peptide (Sigma) prior to LC-MS/MS using the same procedure as above. Aliquots of each sample were loaded onto an Acclaim PepMap 100 precolumn (75 μm×2 cm, C18, 3 μm, 100 Å, Thermo Scientific) in-line with an EASY-Spray, PepMap column (75 μm×50 cm, C18, 2 μm, 100 Å Thermo Scientific) using the autosampler of an EASY-nLC 1000 (Thermo Scientific). Peptides were gradient eluted into a Q Exactive mass spectrometer (Thermo Scientific) using a 60 min gradient from 2% solvent B to 40% solvent B. Solvent A was 2% acetonitrile in 0.5% acetic acid and solvent B was 90% acetonitrile in 0.5% acetic acid. The Q Exactive mass spectrometer was set up to acquire high resolution full MS spectra with a resolution of 70,000 at m/z 200, an AGC target of 1e6, with a maximum ion time of 120 ms, and scan range of 400 to 1500 m/z. Following each full MS twenty data-dependent high resolution HCD MS/MS spectra were acquired using the following instrument parameters: resolution of 17,500 at m/z 200, AGC target of 5e4, maximum ion time of 250 ms, one microscan, 2 m/z isolation window, fixed first mass of 150 m/z, and NCE of 27, dynamic exclusion 30 seconds. For the phosphorylation analysis the MS/MS spectra were searched against a PHLPP2 fasta file using Byonic™ (Bern et al., 2012) and the site of phosphorylation verified by manual inspection of the spectrum. For the binding partner analysis, the MS/MS spectra were searched against the uniprot mouse database using Sequest within Proteome Discoverer (ThermoFisher). The results were filtered using a <1% FDR (False Discovery Rate) searched against a decoy database and excluding proteins with less than two unique peptides.

### Site-Directed Mutagenesis

Site-directed mutagenesis was performed as per the Q5 site-directed mutagenesis kit (NEB) to generate pcDNA3/HA/Flag/PHLPP2-S1119A, -S1210A or -S1119/1210A and resulting plasmids were sequence verified.

### Adenovirus Studies

Ad-GFP and Ad-HA/Flag/PHLPP2 adenoviruses have been described (Kim et al., 2016). Ad-HA/Flag/PHLPP2 (2A) and Ad-shKctd17 were generated by Welgen, Inc. (Worcester, Mass.). For in vivo studies, we injected between 2.5-5×10⁸ purified viral particles per gram body weight, performed metabolic analysis on days 3-7 and killed the mice at day 7 or 10 post-injection.

### Metabolic Analyses

Blood tail vein glucose was measured using a glucose meter (Bayer). Glucose tolerance tests were performed by intraperitoneal injection of 1 or 2 g per kg body weight glucose after a 16 h fast. Hepatic lipids were extracted by the Folch method (Folch et al., 1957), and plasma and hepatic triglyceride (Thermo), Cholesterol E, or NEFA (Wako) measured using colorimetric assays according to the manufacturer's protocol. Plasma insulin concentrations were measured using a mouse insulin ELISA kit (Mercodia).

### Cross-Sectional Analysis of KCTD17 in Human Liver Biopsy Specimens

Liver gene expression were analyzed in individuals who underwent liver biopsy for suspected NASH, due to the presence of persistent elevations in liver enzymes or due to severe obesity (Mancina et al., 2016). The protocol was approved by the Ethical Committee of the Fondazione IRCCS of Milan, and each patient signed a written informed consent. For statistical analysis, comparisons were made by fitting data to generalized linear models, unadjusted (univariate analyses), or considering as independent variables: age, sex, BMI, T2D, PNPLA3 I148M alleles, histological steatosis grade, and activity. Hepatic KCTD17 mRNA levels were normalized for □-actin and log transformed before analyses to ensure a normal distribution.

### RNA/Quantitative PCR

RNA was isolated by TRIzol (Invitrogen) or NucleoSpin RNA (Clontech), and cDNA synthesized with the High-Capacity cDNA Reverse Transcription kit (Applied Biosystems) or SuperScript VILO cDNA synthesis kit (Life Technologies), which was followed by quantitative RT-PCR with Power SYBR Green PCR master mix (Applied Biosystems) in a CFX96 Real-Time PCR detection system (Bio-Rad). Primer sequences are available in Table C.

TABLE C
Quantitative PCR primer sequences

| | Sequences (5′ to 3′) | |
|---|---|---|
| Gene symbol | Forward | Reverse |
| Mouse/Human | AAACGGCTACCACATCCAAG | CCTCCAATGGATCCTCGTTA |
| 18s | | |
| Mouse 36b4 | GTTCTTGCCCATCAGCACC | AGATGCAGCAGATCCGCAT |
| Mouse Fasn | CTGACTCGGCTACTGACACG | TGAGCTGGGTTAGGGTAGGA |
| Mouse G6pc | GTCTGGATTCTACCTGCTAC | AAAGACTTCTTGTGTGTCTGTC |
| Mouse Gcgr | ACGGTACAGCCAGAAGATTG | TCTACCAGCAACCAGCAATAG |
| Mouse Kctd17 | GAGCTCACACAGATGGTATCC | TGGTCCTCACTCCCATAGTT |
| Mouse Pck1 | CCTGGAAGAACAAGGAGTGG | AGGGTCAATAATGGGGCACT |
| Mouse Phlpp1 | AGGGTCCCGGAGACGATAAG | AGGGCGGAGATGTCTTTTGC |
| Mouse Phlpp2 | GCCACAATCTTCTTACAGAGGTC | TCGAGGGGAATGTGCTCCA |
| Mouse | GAAGCTGTCGGGGTAGCGTCT | CTCTCAGGAGAGTTGGCACCTG |
| Srebp1c | | |
| Human ACTB | CATGTACGTTGCTATCCAGGC | CTCCTTAATGTCACGCACGAT |
| Human | CATCGGCTATCCCTACTACTC | TTGAGGGTCCCATTCTCAAAC |
| DSCAML1 | | |
| Human GLIS3 | CAACCAGATCAGTCCTAGCTTAC | CCAAAGACTCACGCGAAATAAG |
| Human JADE1 | CGGGAGCAGGATGTCTTATTT | GGTCACCATGTAAGTGAGGTT |
| Human | AGAGAAGGACTACACGGTCA | CCATCAGACATGGTGGAGAC |
| KCTD17 | | |
| Human | CATACAGGAGGAGGCTACAAATC | GGTCTTTCCCTTGCGTACAT |
| PHLPP2 | | |

### Statistical Analysis

All data shown as mean±s.e.m. ANOVA was used for comparison of means among groups.

### REFERENCES

Ahrens, M. et al. DNA methylation analysis in nonalcoholic fatty liver disease suggests distinct disease-specific and remodeling signatures after bariatric surgery. Cell metabolism 18, 296-302, doi:10.1016/j.cmet.2013.07.004 (2013).
Alessi, D. R., Pearce, L. R., and Garcia-Martinez, J. M. (2009). New insights into mTOR signaling: mTORC2 and beyond. Science signaling 2, pe27.
Ali, S. & Drucker, D. J. Benefits and limitations of reducing glucagon action for the treatment of type 2 diabetes. American journal of physiology. Endocrinblogy and metabolism 296, E415-421, doi:10.1152/ajpendo.90887.2008 (2009).
Andreozzi, F., et al. Increased levels of the Akt-specific phosphatase PH domain leucine-rich repeat protein phosphatase (PHLPP)-1 in obese participants are associated with insulin resistance. Diabetologia 54, 1879-1887 (2011).
Balasubramanian, D. et al. Staphylococcus aureus Coordinates Leukocidin Expression and Pathogenesis by Sensing Metabolic Fluxes via RpiRc. mBio 7, doi:10.1128/mBio.00818-16 (2016).
Beezhold K, Liu J., Kan, H. Meighan T., Castranova V., Shi X., and Chen F. (2011). miR-190-mediated downregulation of PHLPP contributes to arsenic-induced Akt activation and carcinogenesis. Toxicological sciences 123(2), 411-420.
Bern, M., Kil, Y. J. & Becker, C. Byonic: advanced peptide and protein identification software. Current protocols in bioinformatics Chapter 13, Unit13.20, doi:10.1002/0471250953.bi1320540 (2012).
Bhaskar, P. T., and Hay, N. (2007). The two TORCs and Akt. Developmental cell 12, 487-502.
Biddinger, S. B. et al. Hepatic insulin resistance is sufficient to produce dyslipidemia and susceptibility to atherosclerosis. Cell metabolism 7, 125-134, doi:10.1016/j.cmet.2007.11.013 (2008).
Bradley, E. W., Carpio, L. R., and Westendorf, J. J. (2013). Histone deacetylase 3 suppression increases PH domain and leucine-rich repeat phosphatase (Phlpp)1 expression in chondrocytes to suppress Akt signaling and matrix secretion. The Journal of biological chemistry 288, 9572-9582.
Brognard, J., Niederst, M., Reyes, G., Warfel, N., and Newton, A. C. (2009). Common polymorphism in the phosphatase PHLPP2 results in reduced regulation of Akt and protein kinase C. The Journal of biological chemistry 284, 15215-15223.
Brognard, J., Sierecki, E., Gao, T., and Newton, A. C. (2007). PHLPP and a second isoform, PHLPP2, differentially attenuate the amplitude of Akt signaling by regulating distinct Akt isoforms. Molecular cell 25, 917-931.
Brown, N. F., Stefanovic-Racic, M., Sipula, I. J., and Perdomo, G. (2007). The mammalian target of rapamycin regulates lipid metabolism in primary cultures of rat hepatocytes. Metabolism: clinical and experimental 56, 1500-1507.
Butler, P. C. & Rizza, R. A. Contribution to postprandial hyperglycemia and effect on initial splanchnic glucose clearance of hepatic glucose cycling in glucose-intolerant or NIDDM patients. Diabetes 40, 73-81 (1991).
Buzzetti, E., Pinzani, M. & Tsochatzis, E. A. The multiple-hit pathogenesis of non-alcoholic fatty liver disease (NAFLD). Metabolism: clinical and experimental 65, 1038-1048, doi:10.1016/j.metabol.2015.12.012 (2016).
Calvisi, D. F., Wang, C., Ho, C., Ladu, S., Lee, S. A., Mattu, S., Destefanis, G., Delogu, S., Zimmermann, A., Ericsson, J., et al. (2011). Increased lipogenesis, induced by AKT-mTORC1-RPS6 signaling, promotes development of human hepatocellular carcinoma. Gastroenterology 140, 1071-1083.
Conarello, S. L. et al. Glucagon receptor knockout mice are resistant to diet-induced obesity and streptozotocin-mediated beta cell loss and hyperglycaemia. Diabetologia 50, 142-150, doi:10.1007/s00125-006-0481-3 (2007).
Cook, J. R., Matsumoto, M., Banks, A. S., Kitamura, T., Tsuchiya, K., and Accili, D. (2015). A Mutant Allele Encoding DNA-Binding-Deficient Foxo1 Differentially Regulates Hepatic Glucose and Lipid Metabolism. Diabetes.
Cozzone, D., et al. Isoform-specific defects of insulin stimulation of Akt/protein kinase B (PKB) in skeletal muscle cells from type 2 diabetic patients. Diabetologia 51, 512-521 (2008).
Dong, X. C., et al. Inactivation of hepatic Foxo1 by insulin signaling is required for adaptive nutrient homeostasis and endocrine growth regulation. Cell metabolism 8, 65-76 (2008)
Donnelly, K. L. et al. (2005) Sources of fatty acids stored in liver and secreted via lipoproteins in patients with nonalcoholic fatty liver disease. The Journal of clinical investigation 115, 1343-1351, doi:10.1172/JCI23621.
Dowman, J. K., Armstrong, M. J., Tomlinson, J. W., and Newsome, P. N. (2011). Current therapeutic strategies in non-alcoholic fatty liver disease. Diabetes, obesity & metabolism 13, 692-702.
Dunning, B. E. & Gerich, J. E. The role of alpha-cell dysregulation in fasting and postprandial hyperglycemia in type 2 diabetes and therapeutic implications. Endocrine reviews 28, 253-283, doi:10.1210/er.2006-0026 (2007).
Duvel, K., Yecies, J. L., Menon, S., Raman, P., Lipovsky, A. I., Souza, A. L., Triantafellow, E., Ma, Q., Gorski, R., Cleaver, S., et al. (2010). Activation of a metabolic gene regulatory network downstream of mTOR complex 1. Molecular cell 39, 171-183.
Facchinetti, V., Ouyang, W., Wei, H., Soto, N., Lazorchak, A., Gould, C., Lowry, C., Newton, A. C., Mao, Y., Miao, R. Q., et al. (2008). The mammalian target of rapamycin complex 2 controls folding and stability of Akt and protein kinase C. The EMBO journal 27, 1932-1943.
Folch, J., Lees, M., and Sloane Stanley, G. H. (1957). A simple method for the isolation and purification of total 86onalc from animal tissues. The Journal of biological chemistry 226, 497-509.
Ford, E. S., Giles, W. H., and Dietz, W. H. (2002). Prevalence of the metabolic syndrome among US adults: findings from the third National Health and Nutrition Examination Survey. JAMA: the journal of the American Medical Association 287, 356-359.
Gao, M. H., Miyanohara, A., Feramisco, J. R., and Tang, T. (2009). Activation of PH-domain leucine-rich protein phosphatase 2 (PHLPP2) by agonist stimulation in cardiac myocytes expressing adenylyl cyclase type 6. Biochemical and biophysical research communications 384, 193-198.
Gao, T., Furnari, F., and Newton, A. C. (2005). PHLPP: a phosphatase that directly dephosphorylates Akt, promotes apoptosis, and suppresses tumor growth. Molecular cell 18, 13-24.
Gelling, R. W. et al. Lower blood glucose, hyperglucagonemia, and pancreatic alpha cell hyperplasia in glucagon receptor knockout mice. Proceedings of the National Academy of Sciences of the United States of America 100, 1438-1443, doi:10.1073/pnas.0237106100 (2003).
Ghalali, A., Ye, Z. W., Hogberg, J., and Stenius, U. (2014). Phosphatase and Tensin HomoLog Deleted on Chromosome 10 (PTEN) and PH Domain and Leucine-rich Repeat Phosphatase Cross-talk (PHLPP) in Cancer Cells and in Transforming Growth Factor beta-Activated Stem Cells. The Journal of biological chemistry 289, 11601-11615.
Haas, J. T., Miao, J., Chanda, D., Wang, Y., Zhao, E., Haas, M. E., Hirschey, M., Vaitheesvaran, B., Farese, R. V., Jr., Kurland, I. J., et al. (2012). Hepatic insulin signaling is required for obesity-dependent expression of SREBP-1c mRNA but not for feeding-dependent expression. Cell metabolism 15, 873-884.
Grzechnik, A. T. & Newton, A. C. PHLPPing through history: a decade in the life of PHLPP phosphatases. Biochemical Society transactions 44, 1675-1682, doi:10.1042/bst20160170 (2016).
Haeusler, R. A., et al. Integrated control of hepatic lipogenesis versus glucose production requires FoxO transcription factors. Nat Commun 5, 5190 (2014).
Hagiwara, A., Cornu, M., Cybulski, N., Polak, P., Betz, C., Trapani, F., Terracciano, L., Heim, M. H., Ruegg, M. A., and Hall, M. N. (2012). Hepatic mTORC2 activates glycolysis and lipogenesis through Akt, glucokinase, and SREBP1c. Cell metabolism 15, 725-738.
Han, S., Liang, C. P., Westerterp, M., Senokuchi, T., Welch, C. L., Wang, Q., Matsumoto, M., Accili, D., and Tall, A. R. (2009). Hepatic insulin signaling regulates VLDL secretion and atherogenesis in mice. The Journal of clinical investigation 119, 1029-1041.
Hands, S. L., Proud, C. G., and Wyttenbach, A. (2009). mTOR's role in ageing: protein synthesis or autophagy? Aging 1, 586-597.
Harrington, L. S., Findlay, G. M., Gray, A., Tolkacheva, T., Wigfield, S., Rebholz, H., Barnett, J., Leslie, N. R., Cheng, S., Shepherd, P. R., et al. (2004). The TSC1-2 tumor suppressor controls insulin-PI3K signaling via regulation of IRS proteins. The Journal of cell biology 166, 213-223.
Horton, J. D., Goldstein, J. L. & Brown, M. S. SREBPs: activators of the complete program of cholesterol and fatty acid synthesis in the liver. The Journal of clinical investigation 109, 1125-1131, doi:10.1172/JCI15593 (2002).
Howell, J. J. & Manning, B. D. mTOR couples cellular nutrient sensing to organismal metabolic homeostasis. Trends in endocrinology and metabolism: TEM 22, 94-102 (2011).
Frescas, D., and Pagano, M. (2008). Deregulated proteolysis by the F-box proteins SKP2 and beta-TrCP: tipping the scales of cancer. Nature reviews Cancer 8, 438-449.
Ikenoue, T., Inoki, K., Yang, Q., Zhou, X., and Guan, K. L. (2008). Essential function of TORC2 in PKC and Akt turn motif phosphorylation, maturation and 88onalcoho. The EMBO journal 27, 1919-1931.
Inaba, H. et al. Ndell suppresses ciliogenesis in proliferating cells by regulating the trichoplein-Aurora A pathway. The Journal of cell biology 212, 409-423, doi:10.1083/jcb.201507046 (2016).
Jain, A., Arauz, E., Aggarwal, V., Ikon, N., Chen, J., and Ha, T. (2014). Stoichiometry and assembly of mTOR complexes revealed by single-molecule pulldown. Proceedings of the National Academy of Sciences of the United States of America 111, 17833-17838.
Jelinek, L. J. et al. (1993) Expression cloning and signaling properties of the rat glucagon receptor. Science (New York, N.Y.) 259, 1614-1616.
Ji, A. X. et al. Structural Insights into KCTD Protein Assembly and Cullin3 Recognition. Journal of molecular biology 428, 92-107, doi:10.1016/j.jmb.2015.08.019 (2016).
Kasahara, K. et al. Ubiquitin-proteasome system controls ciliogenesis at the initial step of axoneme extension. Nature communications 5, 5081, doi:10.1038/ncomm56081 (2014).
Kaizuka, T., Hara, T., Oshiro, N., Kikkawa, U., Yonezawa, K., Takehana, K., Iemura, S., Natsume, T., and Mizushima, N. (2010). Tti1 and Tel2 are critical factors in mammalian target of rapamycin complex assembly. The Journal of biological chemistry 285, 20109-20116.
Kazierad, D. J. et al. Effects of multiple ascending doses of the glucagon receptor antagonist PF-06291874 in patients with type 2 diabetes mellitus. Diabetes, obesity & metabolism 18, 795-802, doi:10.1111/dom.12672 (2016).
Kelly, R. P. et al. Short-term administration of the glucagon receptor antagonist LY2409021 lowers blood glucose in healthy people and in those with type 2 diabetes. Diabetes, obesity & metabolism 17, 414-422, doi:10.1111/dom.12446 (2015).
Kersten, S. Mechanisms of nutritional and hormonal regulation of lipogenesis. EMBO reports 2, 282-286, doi:10.1093/embo-reports/kve071 (2001).
Kim, D. H., Sarbassov, D. D., Ali, S. M., King, J. E., Latek, R. R., Erdjument-Bromage, H., Tempst, P., and Sabatini, D. M. (2002). mTOR interacts with raptor to form a nutrient-sensitive complex that signals to the cell growth machinery. Cell 110, 163-175.
Kim, D. H., Sarbassov, D. D., Ali, S. M., Latek, R. R., Guntur, K. V., Erdjument-Bromage, H., Tempst, P., and Sabatini, D. M. (2003). GbetaL, a positive regulator of the rapamycin-sensitive pathway required for the nutrient-sensitive interaction between raptor and mTOR. Molecular cell 11, 895-904.
Kim, K., Pyo, S., and Um, S. H. (2012). S6 kinase 2 deficiency enhances ketone body production and increases peroxisome proliferator-activated receptor alpha activity in the liver. Hepatology (Baltimore, Md.) 55, 1727-1737.
Kim, D. H., et al. GbetaL, a positive regulator of the rapamycin-sensitive pathway required for the nutrient-sensitive interaction between raptor and mTOR. Molecular cell 11, 895-904 (2003).
Kim, S., et al. Amino acid signaling to mTOR mediated by inositol polyphosphate multikinase. Cell metabolism 13, 215-221 (2011).
Kim, K. et al. mTORC1-independent Raptor prevents hepatic steatosis by stabilizing PHLPP2. Nat Commun 7, 10255, doi:10.1038/ncomms10255 (2016).
Lefebvre, P. J. Glucagon and its family revisited. Diabetes care 18, 715-730 (1995).
Leslie, N. R., Biondi, R. M., and Alessi, D. R. (2001). Phosphoinositide-regulated kinases and phosphoinositide phosphatases. Chemical reviews 101, 2365-2380.
Li, X., Liu, J. & Gao, T. beta-TrCP-mediated ubiquitination and degradation of PHLPP1 are negatively regulated by Akt. Molecular and cellular biology 29, 6192-6205 (2009).
Li, L., et al. SCD1 Expression is dispensable for hepatocarcinogenesis induced by AKT and Ras oncogenes in mice. PloS one 8, e75104 (2013).
Li, L., Wang, C., Calvisi, D. F., Evert, M., Pilo, M. G., Jiang, L., Yuneva, M., and Chen, X. (2013). SCD1 Expression is dispensable for hepatocarcinogenesis induced by AKT and Ras oncogenes in mice. PloS one 8, e75104.
Li, S., Brown, M. S., and Goldstein, J. L. (2010). Bifurcation of insulin signaling pathway in rat liver: mTORC1 required for stimulation of lipogenesis, but not inhibition of gluconeogenesis. Proceedings of the National Academy of Sciences of the United States of America 107, 3441-3446.
Li, X., Liu, J., and Gao, T. (2009). Beta-TrCP-mediated ubiquitination and degradation of PHLPP1 are negatively regulated by Akt. Molecular and cellular biology 29, 6192-6205.
Li, X., Stevens, P. D., Liu, J., Yang, H., Wang, W., Wang, C., Zeng,
Z., Schmidt, M. D., Yang, M., Lee, E. Y., et al. (2014). PHLPP Is a Negative Regulator of RAF1, Which Reduces Colorectal Cancer Cell Motility and Prevents Tumor Progression in Mice. Gastroenterology 146, 1301-1312.e1310.
Lin, H. V., and Accili, D. (2011). Hormonal regulation of hepatic glucose production in health and disease. Cell metabolism 14, 9-19.
Lu, M., et al. Insulin regulates liver metabolism in vivo in the absence of hepatic Akt and Foxo1. Nature medicine 18, 388-395 (2012). Li X., Liu J. and Gao T. (2009). Beta-TrCP-mediated ubiquitination and degradation of PHLPP1 are negatively regulated by Akt. Molecular and Cellular Biology 29(23), 6192-6205.
Lu, M., Wan, M., Leavens, K. F., Chu, Q., Monks, B. R., Fernandez, S., Ahima, R. S., Ueki, K., Kahn, C. R., and Birnbaum, M. J. (2012). Insulin regulates liver metabolism in vivo in the absence of hepatic Akt and Foxo1. Nature medicine 18, 388-395.
Mancina, R. M. et al. The MBOAT7-TMC4 Variant rs641738 Increases Risk of Nonalcoholic Fatty Liver Disease in Individuals of European Descent. Gastroenterology 150, 1219-1230.e1216, doi:10.1053/j.gastro.2016.01.032 (2016).
Matsumoto, M., Han, S., Kitamura, T. & Accili, D. (2006) Dual role of transcription factor FoxO1 in controlling hepatic insulin sensitivity and lipid metabolism. The Journal of clinical investigation 116, 2464-2472, doi:10.1172/jci27047.
McKnight, G. S. Cyclic AMP second messenger systems. Current opinion in cell biology 3, 213-217 (1991).
Menon, S., Dibble, C. C., Talbott, G., Hoxhaj, G., Valvezan, A. J., Takahashi, H., Cantley, L. C., and Manning, B. D. (2014). Spatial control of the TSC complex integrates insulin and nutrient regulation of mTORC1 at the lysosome. Cell 156, 771-785.
Michael, M. D., et al. Loss of insulin signaling in hepatocytes leads to severe insulin resistance and progressive hepatic dysfunction. Molecular cell 6, 87-97 (2000).
Mishra, N., et al. Efficient hepatic delivery of drugs: novel strategies and their significance. Biomed Res Int 2013, 382184 (2013).
Miyamoto, S., Purcell, N. H., Smith, J. M., Gao, T., Whittaker, R., Huang, K., Castillo, R., Glembotski, C. C., Sussman, M. A., Newton, A. C., et al. (2010). PHLPP-1 negatively regulates Akt activity and survival in the heart. Circulation research 107, 476-484.
Mora, A., Lipina, C., Tronche, F., Sutherland, C. & Alessi, D. R. Deficiency of PDK1 in liver results in glucose intolerance, impairment of insulin-regulated gene expression and liver failure. The Biochemical journal 385, 639-648 (2005).
Newton, A. C., and Trotman, L. C. (2014). Turning off AKT: PHLPP as a drug target. Annual review of pharmacology and toxicology 54, 537-558.
Oh, W. J., Wu, C. C., Kim, S. J., Facchinetti, V., Julien, L. A., Finlan, M., Roux, P. P., Su, B., and Jacinto, E. (2010). mTORC2 can associate with ribosomes to promote cotranslational phosphorylation and stability of nascent Akt polypeptide. The EMBO journal 29, 3939-3951.
Ono, E., et al. Hepatic Akt activation induces marked hypoglycemia, hepatomegaly, and hypertriglyceridemia with sterol regulatory element binding protein involvement. Diabetes 52, 2905-2913 (2003).
Pajvani, U. B., Qiang, L., Kangsamaksin, T., Kitajewski, J., Ginsberg, H. N., and Accili, D. (2013). Inhibition of Notch uncouples Akt activation from hepatic lipid accumulation by decreasing mTorcl stability. Nature medicine 19, 1054-1060.
Pajvani, U. B. & Accili, D. (2015) The new biology of diabetes. Diabetologia 58, 2459-2468, doi:10.1007/s00125-015-3722-5.
Perry, R. J., Samuel, V. T., Petersen, K. F. & Shulman, G. I. The role of hepatic lipids in hepatic insulin resistance and type 2 diabetes. Nature 510, 84-91, doi:10.1038/nature13478 (2014).
Petersen, K. F., Laurent, D., Rothman, D. L., Cline, G. W. & Shulman, G. I. (1998) Mechanism by which glucose and insulin inhibit net hepatic glycogenolysis in humans. The Journal of clinical investigation 101, 1203-1209, doi:10.1172/jci579.
Peterson, T. R., Sengupta, S. S., Harris, T. E., Carmack, A. E., Kang, S. A., Balderas, E., Guertin, D. A., Madden, K. L., Carpenter, A. E., Finck, B. N., et al. (2011). mTOR complex 1 regulates lipin 1 localization to control the SREBP pathway. Cell 146, 408-420.
Peverill, W., Powell, L. W. & Skoien, R. Evolving concepts in the pathogenesis of NASH: beyond steatosis and inflammation. International journal of molecular sciences 15, 8591-8638, doi:10.3390/ijms15058591 (2014).
Porstmann, T., Santos, C. R., Griffiths, B., Cully, M., Wu, M., Leevers, S., Griffiths, J. R., Chung, Y. L., and Schulze, A. (2008). SREBP activity is regulated by mTORC1 and contributes to Akt-dependent cell growth. Cell metabolism 8, 224-236.
Puigserver, P. et al. (2003) Insulin-regulated hepatic gluconeogenesis through FOX01-PGC-1alpha interaction. Nature 423, 550-555, doi:10.1038/nature01667.
Ryu, D. et al. A SIRT7-dependent acetylation switch of GABPbeta1 controls mitochondrial function. Cell metabolism 20, 856-869, doi:10.1016/j.cmet.2014.08.001 (2014).
Saltiel, A. R. & Kahn, C. R. (2001) Insulin 93onalcoho and the regulation of glucose and lipid metabolism. Nature 414, 799-806, doi:10.1038/414799a.
Sarbassov, D. D., Guertin, D. A., Ali, S. M., and Sabatini, D. M. (2005). Phosphorylation and regulation of Akt/PKB by the rictor-mTOR complex. Science (New York, N.Y.) 307, 1098-1101.
Sengupta, S., Peterson, T. R., Laplante, M., Oh, S., and Sabatini, D. M. (2010). mTORC1 controls fasting-induced ketogenesis and its modulation by ageing. Nature 468, 1100-1104.
Shah, O. J., Wang, Z., and Hunter, T. (2004). Inappropriate activation of the TSC/Rheb/mTOR/S6K cassette induces IRS1/2 depletion, insulin resistance, and cell survival deficiencies. Current biology: CB 14, 1650-1656.
Shi, L. et al. (2010) The MicroArray Quality Control (MAQC)-II study of common practices for the development and validation of microarray-based predictive models. Nature biotechnology 28, 827-838, doi:10.1038/nbt.1665.
Shimomura, I. et al. (2000) Decreased IRS-2 and increased SREBP-1c lead to mixed insulin resistance and sensitivity in livers of lipodystrophic, and ob/ob mice. Molecular cell 6, 77-86.
Slawik, M., and Vidal-Puig, A. J. (2006). Lipotoxicity, overnutrition and energy metabolism in aging. Ageing research reviews 5, 144-164.
Sloop, K. W. et al. Hepatic and glucagon-like peptide-1-mediated reversal of diabetes by glucagon receptor antisense oligonucleotide inhibitors. The Journal of clinical investigation 113, 1571-1581, doi:10.1172/jci20911 (2004).
Sorensen, H. et al. Immunoneutralization of endogenous glucagon reduces hepatic glucose output and improves long-term glycemic control in diabetic ob/ob mice. Diabetes 55, 2843-2848, doi:10.2337/db06-0222 (2006).
Stephens, L., Anderson, K., Stokoe, D., Erdjument-Bromage, H., Painter, G. F., Holmes, A. B., Gaffney, P. R., Reese, C. B., McCormick, F., Tempst, P., et al. (1998). Protein kinase B kinases that mediate phosphatidylinositol 3,4,5-trisphosphate-dependent activation of protein kinase B. Science (New York, N.Y.)
Strable, M. S. & Ntambi, J. M. Genetic control of de novo lipogenesis: role in diet-induced obesity. Critical reviews in biochemistry and molecular biology 45, 199-214, doi:10.3109/10409231003667500 (2010).
Subramanian, A. et al. Gene set enrichment analysis: a knowledge-based approach for interpreting genome-wide expression profiles. Proceedings of the National Academy of Sciences of the United States of America 102, 15545-15550, doi:10.1073/pnas.0506580102 (2005).
Summers, S. A. & Birnbaum, M. J. (1997) A role for the serine/threonine kinase, Akt, in insulin-stimulated glucose uptake. Biochemical Society transactions 25, 981-988.
Taniguchi, C. M., Emanuelli, B., and Kahn, C. R. (2006). Critical nodes in 95onalcoho pathways: insights into insulin action. Nature reviews Molecular cell biology 7, 85-96.
Tilg, H. & Moschen, A. R. Evolution of inflammation in nonalcoholic fatty liver disease: the multiple parallel hits hypothesis. Hepatology 52, 1836-1846, doi:10.1002/hep.24001 (2010).
Villanova, N., Moscatiello, S., Ramilli, S., Bugianesi, E., Magalotti, D., Vanni, E., Zoli, M., and Marchesini, G. (2005). Endothelial dysfunction and cardiovascular risk profile in nonalcoholic fatty liver disease. Hepatology (Baltimore, Md.) 42, 473-480.
Wang, J., Cheung, A. T., Kolls, J. K., Starks, W. W., Martinez-Hernandez, A., Dietzen, D., and Bryer-Ash, M. (2001). Effects of adenovirus-mediated liver-selective overexpression of protein tyrosine phosphatase-1b on insulin sensitivity in vivo. Diabetes, obesity & metabolism 3, 367-380.
Wang, P., Zhou, Z., Hu, A., Ponte de Albuquerque, C., Zhou, Y., Hong, L., Sierecki, E., Ajiro, M., Kruhlak, M., Harris, C., et al. (2014). Both Decreased and Increased SRPK1 Levels Promote Cancer by Interfering with PHLPP-Mediated Dephosphorylation of Akt. Molecular cell 54, 378-391.
Wang, X. et al. Hepatocyte TAZ/WWTR1 Promotes Inflammation and Fibrosis in Nonalcoholic Steatohepatitis. Cell metabolism 24, 848-862, doi:10.1016/j.cmet.2016.09.016 (2016).
Williams, E. G. et al. Systems proteomics of liver mitochondria function. Science (New York, N.Y.) 352, aad0189, doi:10.1126/science.aad0189 (2016).
Yabe, D., Brown, M. S., and Goldstein, J. L. (2002). Insig-2, a second endoplasmic reticulum protein that binds SCAP and blocks export of sterol regulatory element-binding proteins. Proceedings of the National Academy of Sciences of the United States of America 99, 12753-12758.
Yabe, D., Komuro, R., Liang, G., Goldstein, J. L., and Brown, M. S. (2003). Liver-specific mRNA for Insig-2 down-regulated by insulin: implications for fatty acid synthesis. Proceedings of the National Academy of Sciences of the United States of America 100, 3155-3160.
Yecies, J. L., et al. Akt stimulates hepatic SREBP1c and lipogenesis through parallel mTORC1-dependent and independent pathways. Cell metabolism 14, 21-32 (2011).
Yecies, J. L., Zhang, H. H., Menon, S., Liu, S., Yecies, D., Lipovsky, A. I., Gorgun, C., Kwiatkowski, D. J., Hotamisligil, G. S., Lee, C. H., et al. (2011). Akt stimulates hepatic SREBP1c and lipogenesis through parallel mTORC1-dependent and independent pathways. Cell metabolism 14, 21-32.
Yip, C. K., Murata, K., Walz, T., Sabatini, D. M., and Kang, S. A. (2010). Structure of the human mTOR complex I and its implications for rapamycin inhibition. Molecular cell 38, 768-774.
Yuan, M., Pino, E., Wu, L., Kacergis, M., and Soukas, A. A. (2012). Identification of Akt-independent regulation of hepatic lipogenesis by mammalian target of rapamycin (mTOR) complex 2. The Journal of biological chemistry 287, 29579-29588.
Zhang, Y. L., Hernandez-Ono, A., Siri, P., Weisberg, S., Conlon, D., Graham, M. J., Crooke, R. M., Huang, L. S., and Ginsberg, H. N. (2006). Aberrant hepatic expression of PPARgamma2 stimulates hepatic lipogenesis in a mouse model of obesity, insulin resistance, dyslipidemia, and hepatic steatosis. The Journal of biological chemistry 281, 37603-37615.

What is claimed:

1. A method of reducing a subject's hepatic and plasma triglyceride levels comprising administering to a subject in need thereof a pharmaceutical composition comprising a pharmaceutical carrier and a compound that decreases KCTD17 expression in liver cells in an amount effective to reduce the subject's hepatic and plasma triglyceride levels.

2. A method of reducing a subject's hepatic and plasma triglyceride levels comprising administering to a subject in need thereof a pharmaceutical composition comprising a pharmaceutical carrier and a compound that prevents PHLPP2 degradation in liver cells in an amount effective to reduce the subject's hepatic and plasma triglyceride levels.

3. The method of claim 2, wherein the pharmaceutical composition inhibits Glucagon signaling.

4. A method of reducing a subject's hepatic and plasma triglyceride levels comprising administering to a subject in need thereof a pharmaceutical composition comprising a pharmaceutical carrier and a compound that inhibits Glucagon signaling in liver cells in an amount effective to reduce the subject's hepatic and plasma triglyceride levels.

5. The method of claim 4, wherein the pharmaceutical composition reduces PHLPP2 degradation.

6. The method of claim 4 or 5, wherein the pharmaceutical composition increases free Raptor in the liver cells.

7. The method of claim 1-6, wherein the pharmaceutical composition decreases PHLPP2 phosphorylation at Serine 1119 and Serine 1210 residues in liver cells.

8. The method of claim 6, wherein the pharmaceutical composition prevents PHLPP2 degradation in liver cells.

9. The method of any one of claims 1-8, wherein the pharmaceutical composition increases PHLPP2 in liver cells.

10. The method of any one of claim 3-9, wherein the pharmaceutical composition prevents PHLPP2 degradation in liver cells.

11. The method of any one of claims 1-10, wherein the pharmaceutical composition decreases Akt phosphorylation at Serine 473 residue in liver cells.

12. The method of any one of claims 1-11, wherein the pharmaceutical composition decreases Akt signaling.

13. The method of any one of claims 1-12, wherein the pharmaceutical composition increases Raptor expression, thereby increasing free Raptor in the liver cells.

14. The method of any one of claims 1-13, wherein the pharmaceutical composition inhibits interaction of Raptor and mTORC1, thereby increasing free Raptor in the liver cells.

15. The method of any one of claims 1-14, wherein the compound reduces the expression of at least one lipogenic gene.

16. The method of claim 15, wherein the at least one lipogenic gene is Srebp1c, Fasn, Acc1, or Scd1.

17. The method of any one of claims 1-16, wherein the subject is afflicted with a metabolic disease.

18. The method of any one of claims 1-17, wherein the pharmaceutical composition comprises a polynucleotide.

19. The method of any one of claims 1-18, wherein the pharmaceutical composition is targeted to the liver of the subject.

20. The method of claim 17, wherein the metabolic disease is obesity.

21. The method of claim 17, wherein the metabolic disease is hypertriglyceridemia.

22. The method of claim 17, wherein the metabolic disease is hyperinsulinemia.

23. The method of claim 17, wherein the metabolic disease is Type 2 Diabetes.

24. The method of claim 17, wherein the metabolic disease is fatty liver disease.

25. The method of claim 24, wherein the fatty liver disease is nonalcoholic fatty liver disease or 100onalcoholic steatohepatitis.

26. The method of any one of claims 1-25, wherein the subject is afflicted with cirrhosis or hepatocellular carcinoma.

27. The method of any one of claims 1-26, wherein the subject is a human.

28. The method of any one of claims 1-27, wherein the subject's hepatic or plasma triglyceride levels are >150 mg/dL.

29. The method of any one of claims 1-28, wherein the subject's hepatic or plasma triglyceride levels are >500 mg/dL, about 200 to 499 mg/dL, or about 150 to 199 mg/dL.

30. The method of any one of claims 1-29, wherein the subject's hepatic or plasma triglyceride levels are reduced by at least 20%, at least 25%, at least 30%, at least 35%, at least 40%, at least 45%, at least 50%, at least 55%, at least 60%, at least 65%, at least 70%, or at least 75%, relative to the level prior to the administration.

31. A process for determining the amount of KCTD17 expression in a subject's liver comprising:
a) obtaining a biological sample comprising liver cells of the subject;
b) determining the amount of KCTD17 mRNA in the sample.

32. A process for diagnosing whether a subject is afflicted with increased KCTD17 expression comprising:
a) determining the amount of KCTD17 in the subject according to the process of claim 31;
b) determining the amount of KCTD17 in a reference subject according to the process of claim 31; and
c) diagnosing the subject to be afflicted with increased KCTD17 expression if the amount of KCTD17 expression in step (a) is substantially increased compared to the amount of KCTD17 expression in step (b).

33. A method of treating a subject diagnosed to be afflicted with increased KCTD17 expression according to the process of claim 32 comprising reducing the subject's hepatic and plasma triglyceride levels according to the method of claim 1, 3, 6 or 7.

34. A method of treating a subject afflicted with elevated triglyceride levels comprising administering to the subject a pharmaceutical composition comprising a pharmaceutical carrier and a compound that decreases KCTD17 expression in liver cells in an amount effective to reduce the subject's hepatic and plasma triglyceride levels.

35. A method of treating a subject afflicted with elevated triglyceride levels comprising administering to the subject a pharmaceutical composition comprising a pharmaceutical carrier and a compound that decreases PHLPP2 phosphorylation at Serine 1119 and Serine 1210 residues in liver cells in an amount effective to reduce the subject's hepatic and plasma triglyceride levels.

36. A method of treating a subject at risk of developing elevated triglyceride levels comprising administering to the subject a pharmaceutical composition comprising a pharmaceutical carrier and a compound that decreases KCTD17 expression in liver cells in an amount effective to reduce the subject's hepatic and plasma triglyceride levels.

37. A method of treating a subject at risk of developing elevated triglyceride levels comprising administering to the subject a pharmaceutical composition comprising a pharmaceutical carrier and a compound that increases PHLPP2 phosphorylation at Serine 1119 and Serine 1210 residues in liver cells in an amount effective to reduce the subject's hepatic and plasma triglyceride levels.


SEQUENCE LISTING (4 sequences)

SEQ ID NO: 1 - protein, 1335 aa, Homo sapiens
MESEMLQSPLLGLGEEDEADLTDWNLPLAFMKKRHCEKIEGSKSLAQSWRMKDRMKTVSVALVLCLNVGVDPPDVVKTTPCARLECWIDPLSMGPQKALETIGANLQKQYENWQPRARYKQSLDPTVDEVKKLCTSLRRNAKEERVLFHYNGHGVPRPTVNGEVWVFNKNYTQYIPLSIYDLQTWMGSPSIFVYDCSNAGLIVKSFKQFALQREQELEVAAINPNHPLAQMPLPPSMKNCIQLAACEATELLPMIPDLPADLFTSCLTTPIKIALRWFCMQKCVSLVPGVTLDLIEKIPGRLNDRRTPLGELNWIFTAITDTIAWNVLPRDLFQKLFRQDLLVASLFRNFLLAERIMRSYNCTPVSSPRLPPTYMHAMWQAWDLAVDICLSQLPTIIEEGTAFRHSPFFAEQLTAFQVWLTMGVENRNPPEQLPIVLQVLLSQVHRLRALDLLGRFLDLGPWAVSLALSVGIFPYVLKLLQSSARELRPLLVFIWAKILAVDSSCQADLVKDNGHKYFLSVLADPYMPAEHRTMTAFILAVIVNSYHTGQEACLQGNLIAICLEQLNDPHPLLRQWVAICLGRIWQNFDSARWCGVRDSAHEKLYSLLSDPIPEVRCAAVFALGTFVGNSAERTDHSTTIDHNVAMMLAQLVSDGSPMVRKELVVALSHLVVQYESNFCTVALQFIEEEKNYALPSPATTEGGSLTPVRDSPCTPRLRSVSSYGNIRAVATARSLNKSLQNLSLTEESGGAVAFSPGNLSTSSSASSTLGSPENEEHILSFETIDKMRRASSYSSLNSLIGVSFNSVYTQIWRVLLHLAADPYPEVSDVAMKVLNSIAYKATVNARPQRVLDTSSLTQSAPASPTNKGVHIHQAGGSPPASSTSSSSLTNDVAKQPVSRDLPSGRPGTTGPAGAQYTPHSHQFPRTRKMFDKGPEQTADDADDAAGHKSFISATVQTGFCDWSARYFAQPVMKIPEEHDLESQIRKEREWRFLRNSRVRRQAQQVIQKGITRLDDQIFLNRNPGVPSVVKFHPFTPCIAVADKDSICFWDWEKGEKLDYFHNGNPRYTRVTAMEYLNGQDCSLLLTATDDGAIRVWKNFADLEKNPEMVTAWQGLSDMLPTTRGAGMVVDWEQETGLLMSSGDVRIVRIWDTDREMKVQDIPTGADSCVTSLSCDSHRSLIVAGLGDGSIRVYDRRMALSECRVMTYREHTAWVVKASLQKRPDGHIVSVSVNGDVRIFDPRMPESVNVLQIVKGLTALDIHPQADLIACGSVNQFTAIYNSSGELINNIKYYDGFMGQRVGAISCLAFHPHWPHLAVGSNDYYISVYSVEKRVR

SEQ ID NO: 2 - protein, 1323 aa, Homo sapiens
MKRNGSRNCLNRRSRFGSRERDWLREDVKRGCVYLYGADTTTATTTTTTSSSSSSSSSSSDLHLVLCTVETPASEICAGEGRESLYLQLHGDLVRRLEPTERPLQIVYDYLSRLGFDDPVRIQEEATNPDLGCMIRFYGEKPCHMDRLDRILLSGIYNVRKGKTQLHKWAERLVVLCGTCLIVSSVKDCQTGKMHILPLVGGKIEEVKRRQYSLAFSSAGAQAQTYHVSFETLAEYQRWQRQASKVVSQRISTVDLSCYSLEEVPEHLFYSQDITYLNLRHNFMQLERPGGLDTLYKFSQLKGLNLSHNKLGLFPILLCEISTLTELNLSCNGFHDLPSQIGNLLNLQTLCLDGNFLTTLPEELGNLQQLSSLGISFNNFSQIPEVYEKLTMLDRVVMAGNCLEVLNLGVLNRMNHIKHVDLRMNHLKTMVIENLEGNKHITHVDLRDNRLTDLDLSSLCSLEQLHCGRNQLRELTLSGFSLRTLYASSNRLTAVNVYPVPSLLTFLDLSRNLLECVPDWACEAKKIEVLDVSYNLLTEVPVRILSSLSLRKLMLGHNHVQNLPTLVEHIPLEVLDLQHNALTRLPDTLFSKALNLRYLNASANSLESLPSACTGEESLSMLQLLYLTNNLLTDQCIPVLVGHLHLRILHLANNQLQTFPASKLNKLEQLEELNLSGNKLKTIPTTIANCKRLHTLVAHSNNISIFPEILQLPQIQFVDLSCNDLTEILIPEALPATLQDLDLTGNTNLVLEHKTLDIFSHITTLKIDQKPLPTTDSTVTSTFWSHGLAEMAGQRNKLCVSALAMDSFAEGVGAVYGMFDGDRNEELPRLLQCTMADVLLEEVQQSTNDTVFMANTFLVSHRKLGMAGQKLGSSALLCYIRPDTADPASSFSLTVANVGTCQAVLCRGGKPVPLSKVFSLEQDPEEAQRVKDQKAIITEDNKVNGVTCCTRMLGCTYLYPWILPKPHISSTPLTIQDELLILGNKALWEHLSYTEAVNAVRHVQDPLAAAKKLCTLAQSYGCQDNVGAMVVYLNIGEEGCTCEMNGLTLPGPVGFASTTTIKDAPKPATPSSSSGIASEFSSEMSTSEVSSEVGSTASDEHNAGGLDTALLPRPERRCSLHPTPTSGLFQRQPSSATFSSNQSDNGLDSDDDQPVEGVITNGSKVEVEVDIHCCRGRDLENSPPLIESSPTLCSEEHARGSCFGIRRQNSVNSGMLLPMSKDRMELQKSPSTSCLYGKKLSNGSIVPLEDSLNLIEVATEVPKRKTGYFAAPTQMEPEDQFVVPHDLEEEVKEQMKQHQDSRLEPEPHEEDRTEPPEEFDTAL

SEQ ID NO: 3 - DNA, 1294 bp, Homo sapiens
gcatagaatgcagatgagcaaagtgagtgggagagggaagtcatttgtaacaaaaactcattatttacagatgagaaatttatattgtcagcgtaatatctgtgaggctaaacagagctggagagtatataaaagcagtgcgccttggtgcagaagtacagagcttaggacacagagcacatcaaaagttcccaaagagggcttgctctctcttcacctgctctgttctacagcacactaccagaagacagcagaaatgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaaagatacaattaaataaaattttcaagcttctttaccattgtct

SEQ ID NO: 4 - DNA, 2034 bp, Homo sapiens
ggatctggcagcgccgcgaagacgagcggtcaccggcgcccgacccgagcgcgcccagaggacggcggggagccaagccgacccccgagcagcgccgcgcgggccctgaggctcaaaggggcagcttcaggggaggacaccccactggccaggacgccccaggctctgctgctctgccactcagctgccctcggaggagcgtacacacacaccaggactgcattgccccagtgtgcagcccctgccagatgtgggaggcagctagctgcccagaggcatgcccccctgccagccacagcgacccctgctgctgttgctgctgctgctggcctgccagccacaggtcccctccgctcaggtgatggacttcctgtttgagaagtggaagctctacggtgaccagtgtcaccacaacctgagcctgctgccccctcccacggagctggtgtgcaacagaaccttcgacaagtattcctgctggccggacacccccgccaataccacggccaacatctcctgcccctggtacctgccttggcaccacaaagtgcaacaccgcttcgtgttcaagagatgcgggcccgacggtcagtgggtgcgtggaccccgggggcagccttggcgtgatgcctcccagtgccagatggatggcgaggagattgaggtccagaaggaggtggccaagatgtacagcagcttccaggtgatgtacacagtgggctacagcctgtccctgggggccctgctcctcgccttggccatcctggggggcctcagcaagctgcactgcacccgcaatgccatccacgcgaatctgtttgcgtccttcgtgctgaaagccagctccgtgctggtcattgatgggctgctcaggacccgctacagccagaaaattggcgacgacctcagtgtcagcacctggctcagtgatggagcggtggctggctgccgtgtggccgcggtgttcatgcaatatggcatcgtggccaactactgctggctgctggtggagggcctgtacctgcacaacctgctgggcctggccaccctccccgagaggagcttcttcagcctctacctgggcatcggctggggtgcccccatgctgttcgtcgtcccctgggcagtggtcaagtgtctgttcgagaacgtccagtgctggaccagcaatgacaacatgggcttctggtggatcctgcggttccccgtcttcctggccatcctgatcaacttcttcatcttcgtccgcatcgttcagctgctcgtggccaagctgcgggcacggcagatgcaccacacagactacaagttccggctggccaagtccacgctgaccctcatccctctgctgggcgtccacgaagtggtctttgccttcgtgacggacgagcacgcccagggcaccctgcgctccgccaagctcttcttcgacctcttcctcagctccttccagggcctgctggtggctgtcctctactgcttcctcaacaaggaggtgcagtcggagctgcggcggcgttggcaccgctggcgcctgggcaaagtgctatgggaggagcggaacaccagcaaccacagggcctcatcttcgcccggccacggccctcccagcaaggagctgcagtttgggaggggtggtggcagccaggattcatctgcggagacccccttggctggtggcctccctagattggctgagagccccttctgaaccctgctgggaccccagctagggctggactctggcacccagaggcgtcgctggacaacccagaactggacgcccagctgaggctgggggcgggggagccaacagcagcccccacctaccccccacccccagtgtggctgtctgcgagattgggcctcctctccctgcacctgccttgtccctggtgcagaggtgagcagaggagtccagggcgggagtgggggctgtgccgtgaactgcgtgccagtgtccccacgtatgtcggcacgtcccatgtgcatggaaatgtcctccaacaataaagagctcaagtggtcaccgtg